SEQUENCE LISTING (97 sequences)

SEQ ID NO: 1 - protein, 215 aa, Artificial Sequence
  Synthetic
MDYKDDDDKGIHGVPAAAEAKEENREKNEEEESVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADTERERHTRRLQKAQ

SEQ ID NO: 2 - DNA, 648 bp, Artificial Sequence
  Synthetic
atggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaataa

SEQ ID NO: 3 - protein, 224 aa, Artificial Sequence
  Synthetic
MDYKDDDDKGIHGVPAAAEAKEENREKNEEEESVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADTERERHTRRLQKAQGGSGGGSGG

SEQ ID NO: 4 - DNA, 672 bp, Artificial Sequence
  Synthetic
atggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggagga

SEQ ID NO: 5 - RNA, 7 nt, Artificial Sequence
  Synthetic
uauguau

SEQ ID NO: 6 - protein, 279 aa, Artificial Sequence
  Synthetic
AIIKPNRDLKPDGVTVVTREVWAENLESEFELISEIIDDYPFISMDTEFPGVIFKSDLRFTNPDDLYTLLKANVDALSLIQVGLTLSDVNGNLPDLGDDLHRGFIWEFNFRDFDVARDAHAPDSIELLRRQGIDFERNCRDGVESERFAELMMSSGLVCNEEVSWVTFHSAYDFGYLMKILTRRELPGALGEFKRVMRVLFGERVYDVKHMMKFCERRLFGGLDRVARTLEVNRAVGKCHQAGSDSLLTWHAFQRMRDLYFVQDGPEKHAGVLYGLEVF

SEQ ID NO: 7 - DNA, 840 bp, Artificial Sequence
  Synthetic
gcgatcattaaaccaaaccgagatctcaaaccggacggtgtcacagtcgtgactcgtgaagtctgggcggagaatctcgaatcagaatttgaactaatcagcgaaatcatcgacgattatccatttatctcaatggacacagaatttcccggcgtgattttcaaatccgatctccggtttacgaatcccgacgatctttatactcttcttaaagctaacgtcgacgctctcagcttaatccaagtcggtctcactctctctgatgttaacggtaacctcccagacctcggtgacgatcttcaccgaggattcatctgggagtttaatttccgtgacttcgacgtggcgcgtgacgcacacgcgcctgattcgatcgagttgcttcgtcggcaaggtatcgattttgagcggaattgtcgcgacggtgttgagtcggagaggtttgcggagctgatgatgtcgtcggggcttgtttgtaatgaggaagtgagctgggtgacgtttcatagcgcgtatgatttcggttacttgatgaagattctcacgcgccgagaattgcccggcgcgttgggggagtttaagcgcgtgatgagagtgttgtttggggagcgtgtgtatgatgtgaagcatatgatgaagttttgtgaaaggagattgtttggtggtttggaccgggttgcaaggacgcttgaggttaaccgggcggttgggaaatgtcatcaagctggttcggatagtttgcttacgtggcatgcgtttcagaggatgagggatttgtattttgttcaagatgggccggagaaacatgctggggttttgtatgggcttgaggttttttaa

SEQ ID NO: 8 - protein, 503 aa, Artificial Sequence
  Synthetic
MDYKDDDDKGIHGVPAAAEAKEENREKNEEEESVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADTERERHTRRLQKAQGGSGGGSGGAIIKPNRDLKPDGVTVVTREVWAENLESEFELISEIIDDYPFISMDTEFPGVIFKSDLRFTNPDDLYTLLKANVDALSLIQVGLTLSDVNGNLPDLGDDLHRGFIWEFNFRDFDVARDAHAPDSIELLRRQGIDFERNCRDGVESERFAELMMSSGLVCNEEVSWVTFHSAYDFGYLMKILTRRELPGALGEFKRVMRVLFGERVYDVKHMMKFCERRLFGGLDRVARTLEVNRAVGKCHQAGSDSLLTWHAFQRMRDLYFVQDGPEKHAGVLYGLEVF

SEQ ID NO: 9 - DNA, 1512 bp, Artificial Sequence
  Synthetic
atggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggagcgatcattaaaccaaaccgagatctcaaaccggacggtgtcacagtcgtgactcgtgaagtctgggcggagaatctcgaatcagaatttgaactaatcagcgaaatcatcgacgattatccatttatctcaatggacacagaatttcccggcgtgattttcaaatccgatctccggtttacgaatcccgacgatctttatactcttcttaaagctaacgtcgacgctctcagcttaatccaagtcggtctcactctctctgatgttaacggtaacctcccagacctcggtgacgatcttcaccgaggattcatctgggagtttaatttccgtgacttcgacgtggcgcgtgacgcacacgcgcctgattcgatcgagttgcttcgtcggcaaggtatcgattttgagcggaattgtcgcgacggtgttgagtcggagaggtttgcggagctgatgatgtcgtcggggcttgtttgtaatgaggaagtgagctgggtgacgtttcatagcgcgtatgatttcggttacttgatgaagattctcacgcgccgagaattgcccggcgcgttgggggagtttaagcgcgtgatgagagtgttgtttggggagcgtgtgtatgatgtgaagcatatgatgaagttttgtgaaaggagattgtttggtggtttggaccgggttgcaaggacgcttgaggttaaccgggcggttgggaaatgtcatcaagctggttcggatagtttgcttacgtggcatgcgtttcagaggatgagggatttgtattttgttcaagatgggccggagaaacatgctggggttttgtatgggcttgaggttttttaa

SEQ ID NO: 10 - DNA, 12891 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 11 - protein, 250 aa, Arabidopsis thaliana
MKFNVANPTTGCQKKLEIDDDQKLRAFYDKRISQEVSGDALGEEFKGYVFKIKGGCDKQGFPMKQGVLTPGRVRLLLHRGTPCFRGHGRRTGERRRKSVRGCIVSPDLSVLNLVIVKKGENDLPGLTDTEKPRMRGPKRASKIRKLFNLKKEDDVRTYVNTYRRKFTNKKGKEVSKAPKIQRLVTPLTLQRKRARIADKKKKIAKANSDAADYQKLLASRLKEQRDRRSESLAKKRSRLSSAAAKPSVTA

SEQ ID NO: 12 - DNA, 1848 bp, Arabidopsis thaliana
aaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacg

SEQ ID NO: 13 - protein, 475 aa, Artificial Sequence
  Synthetic
MDYKDDDDKGIHGVPAAAEAKEENREKNEEEESVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADTERERHTRRLQKAQGGSGGGSGGGSKFNVANPTTGCQKKLEIDDDQKLRAFYDKRISQEVSGDALGEEFKGYVFKIKGGCDKQGFPMKQGVLTPGRVRLLLHRGTPCFRGHGRRTGERRRKSVRGCIVSPDLSVLNLVIVKKGENDLPGLTDTEKPRMRGPKRASKIRKLFNLKKEDDVRTYVNTYRRKFTNKKGKEVSKAPKIQRLVTPLTLQRKRARIADKKKKIAKANSDAADYQKLLASRLKEQRDRRSESLAKKRSRLSSAAAKPSVTA

SEQ ID NO: 14 - DNA, 2084 bp, Artificial Sequence
  Synthetic
atggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaa

SEQ ID NO: 15 - DNA, 4595 bp, Artificial Sequence
  Synthetic
gatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 16 - DNA, 1307 bp, Artificial Sequence
  Synthetic
atcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagg

SEQ ID NO: 17 - DNA, 709 bp, Artificial Sequence
  Synthetic
cctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 18 - DNA, 2753 bp, Artificial Sequence
  Synthetic
gatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatcctccggacctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 19 - DNA, 49 bp, Artificial Sequence
  Synthetic
ggatcctccggacctaggtctagagagttaattaagacccgggactagt

SEQ ID NO: 20 - DNA, 1308 bp, Artificial Sequence
  Synthetic
gatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagg

SEQ ID NO: 21 - DNA, 709 bp, Artificial Sequence; identical to SEQ ID NO: 17
  Synthetic

SEQ ID NO: 22 - protein, 8 aa, Artificial Sequence
  Synthetic
GIHGVPAA

SEQ ID NO: 23 - DNA, 24 bp, Artificial Sequence
  Synthetic
ggcatccacggggtgccagctgct

SEQ ID NO: 24 - protein, 9 aa, Artificial Sequence
  Synthetic
GGSGGGSGG

SEQ ID NO: 25 - DNA, 27 bp, Artificial Sequence
  Synthetic
ggaggatctggaggaggatctggagga

SEQ ID NO: 26 - protein, 8 aa, Artificial Sequence
  Synthetic
DYKDDDDK

SEQ ID NO: 27 - DNA, 24 bp, Artificial Sequence
  Synthetic
gattataaggacgatgacgataaa

SEQ ID NO: 28 - protein, 13 aa, Artificial Sequence
  Synthetic
  misc_feature (11)..(11): Xaa can be any naturally occurring amino acid
RKGFYGAFYVXFY

SEQ ID NO: 29 - protein, 10 aa, Artificial Sequence
  Synthetic
  misc_feature (7)..(7): Xaa can be any naturally occurring amino acid
LIFYVIXNGL

SEQ ID NO: 30 - protein, 441 aa, Arabidopsis thaliana
MAEAKEENREKNEEEESVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADTERERHTRRLQKAQSHIARLGNGDPTNPSLFGALPMGYVPPYNGYGYHQPPGTYGYMLPPIQNQAAFSNMIAQPNQGNNNALQGTSPDSVPPRLARRNFPMPPGNYMGSGYPAMRGHPFPFAYPRGIVSPRPLSSSPGSISPGMSTPLGIGLSSVVQTEGPEGANLFIYNIPREFGDQELAAAFQSFGIVLSAKVFVDKATGVSKCFGFVSYDSQAAAQNAIDMMNGRHLGGKKLKVQLKRDSNNGQPSSNPSLIS

SEQ ID NO: 31 - DNA, 1326 bp, Artificial Sequence
  Synthetic
atggcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaatctcacatcgcccgactagggaacggtgatccaacaaatccctcattgtttggagcattacctatgggttatgtaccaccatataatggatatggttatcatcagcctcctggaacttatggttacatgctaccaccaattcagaaccaagctgcattttccaatatgattgcacaaccaaaccaaggtaataacaatgcattgcaaggaacatcgcctgactctgtgccgcctcgtttggcccgtagaaactttcctatgcctcctggaaactacatgggatctggttatcctgctatgcgaggtcatccttttccatttgcttatcccagaggaattgttagtcctcgccctctaagtagctctcctggatctatatcacctggcatgtcaacacctttaggcattggtttgagttctgtggttcaaaccgaaggtccagaaggtgcaaatctgtttatctataacatacctcgggagttcggggatcaagaactcgcggctgcatttcaatctttcggtattgttctgagtgccaaggtttttgtagacaaagccactggtgtaagcaaatgtttcggttttgttagctatgactcacaagccgcggcgcagaacgctattgacatgatgaatggtcgccatttaggcggtaagaaattgaaagtccagcttaagagagacagcaacaatggccaaccgagtagtaatccttccttaatctcctga

SEQ ID NO: 32 - DNA, 3461 bp, Arabidopsis thaliana
aaaaacaaaacaatacaggtttgactacaaatacacattttcgaacagatagatagagagagaaaaagaaacacaataaaacgcgcgtaaaaaggaaggctaatttagtctttttcgtcgtaactcccataacgagacaaaacaatcttaagtgttccctctcatcgtaaaagcttttctccttccttctatgtacagctgtaatggcaatatcgtaaataactcgtaacagaaagggcaattattgtcaacatttataatttaatttctcccaacttctcaaattccaccatttttcttcatcctctcctccataattgattttccttttttttgtttttccttcttgcaactaaaataaataaatttttagaaaccaaaaaaaaaattgtagagagagaaaaaaaaaactcagaggagagaaagagaagacgacgaagacgaccattttttttttttttgtcaagagagaagacggagacaacaatggcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctctcgaggtttccttttctttttttttaatcttcttcacgcctctgttttcgatttttttttttataaagtttttgattggagaatgtggtgtttgattcgtgtgatttgttgagatttatttgaatttccgaaaccctaatctgtgtttttttttgttaatcttccgattttcaggatgttgttttctgctatgtccatcgagagaagaagcagataagttggtcaatgcttgccataacaagaagacattgcctggggtttgttttttttttgttttttttttcatttctctgactatttgttcttctttcagatgcttgaatgtgatttgatgttctttagtttttaattattggatttcctcattcgcacatttttttttttcgttgagaatctcgaggtttttcttaaaaaatgttattcttaaatctggtttttggaactgcattgttttggtttctgttggattccagaattggcaaattctgattttggatatatgttttgcacgaagcatttctttctgtttaggtgaacatcaatttcttttgcccgaatcagaatcagaatctttcgatttaagagtctgtttcgttggagtcttattgacattgttttggaattgtttcttcaaacccaagaaagtgcaatcaatggtaaaactaggaaattgctataatgagattaatgcgaaagaaacacttttggtttactctttaatagaagcaaaatgttagtttgatccttgttcttatggaaacctgaaaaatgttggcattagtgtttttttaccatggttgtttcagtgtgttcttcggccttttgactgtaacatttaggatgcttagatccttgtcaggtctctaactaactttttcttttggttgctcaggctaacagtctattgcaagtaaagtatgcagatggcgaattagaaaggctaggtaacgcgattccgtgctctatttcacccattttcattgttttatcatcaactgatggatgttgtattaatgtattctgcaatccagagcataagctatttgttggtatgcttccaaagaatgtctctgaagctgaagttcaatctttattctccaagtacgggaccataaaggatctacagattctaagaggtgctcaacaaacgagcaaaggttgaatctatctctgtgtcgctattgtgatctttggtttcactagtttgctttacctagttttgaccttttgtttgttgtcgatgtttaggctgtgcatttctgaagtatgagacaaaagaacaagctgtttccgccatggaatctatcaacggaaaacataaaatggaggtaattgcttttgctactttgaaaacttaccgtttgtaaatctgtagtgtgaaaattccatcatttttcttttggttttcttcttagggttcaactgttcctttagttgtcaaatgggcagacacagaacgagaaagacacacaagaagacttcaaaaggctcaatctcacatcgcccgactagggaacggtgatccaacaaatccctcattgtttggagcattacctatgggttatgtaccaccatataatggatatggttatcatgtaagcaagaaagcctaatactccgttagttcaagctgtataagatttgtctttatcaatcttgtaactaataaaattttcgtttccatttccatagcagcctcctggaacttatggttacatgctaccaccaattcagaaccaagctgcattttccaatatgattgcacaaccaaaccaaggtaataacaatgcattgcaaggaacatcgcctgactctgtgccgcctcgtttggcccgtagaaactttcctatgcctcctggaaactacatgggatctggttatcctgctatgcgaggtcatccttttccatttgcttatcccagaggaattgttagtcctcgccctctaagtagctctcctggatctatatcacctggcatgtcaacacctttaggcattggtttgagttctgtggttcaaaccgaaggttagtgtaattaagacctcttaatagttttcttctcttattcttttaagacctgaaagattattttggttgttcaggtccagaaggtgcaaatctgtttatctataacatacctcgggagttcggggatcaagaactcgcggctgcatttcaatctttcggtattgttctgagtgccaaggtttttgtagacaaagccactggtgtaagcaaatgtttcggtaagctctcttttgatctagtttttcctcttttaaaactggttcattgtgaaggtagaaagaaagcattcttttgctgttgctgttggttgcaggttttgttagctatgactcacaagccgcggcgcagaacgctattgacatgatgaatggtcgccatttaggcggtaagaaattgaaagtccagcttaagagagacagcaacaatggccaaccgagtagtaatccttccttaatctcctgaaagtgctaaaactcctcctctcttcaacttgaaagttcataacagaaaaaaacaaaaaacatattcgggcttatgggtcgacccggttcaaagatttggaccgatcttgaatggatgcatctgtatagatccaaatctcattgtgggtcaaaatgtcccatcaactcaaaagctccaaactggatcatctcttttctttttttttttaatctgcaaataacaatttgtaatttatgtttattattgaaactcatgtagaaattttatgatttattaacattaaattcattgaattcttcttctatcatttt

SEQ ID NO: 33 - protein, 199 aa, Artificial Sequence
  Synthetic
MAEAKEENREKNEEEESVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADTERERHTRRLQKAQ

SEQ ID NO: 34 - DNA, 600 bp, Artificial Sequence
  Synthetic
atggcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaataa

SEQ ID NO: 35 - RNA, 1431 nt, Arabidopsis thaliana
ugagguuucaagcguccaucauauuugcgacacauaugacugacuauuuagcuccucccucuuucuuucucuuauuuuauuaucuuucuccaagaaauaaaauagaaaagaaaauauauaugguuucacaaacaccauuaccauaacuacaacgagaagaggaucuuuuuuaaggagaaaagcagagagagaagagacgagugugugaaguuuuuuugucuuuuguuucuuuuauuacacacaaauagaugaaacgaggaaagcuacuucuuuugcuacuuccauaaaaagguucuuccuuucgcagagaaucaacuuugaucaucuucuuccuucucuuucuuucuucuucucccuccaguaaugcuuauauagucuccuccuauaucucuaccuauacauacacaaacccuuuauccucgaaagcuuccuccugguuagguuuuuaucaaacccuuuuagccaaucgggaaauuaacuaaagaagaagauauggugaggggcaaaacucagaugaagagaauagagaaugcaacaagcagacaagugacuuucuccaaaagaaggaaugguuuguugaagaaagccuuugagcucucagugcuuugugaugcugaaguuucucuuaucaucuucucuccuaaaggcaaacuuuaugaauucgccagcuccaauaugcaagauaccauagaucguuaucugaggcauacuaaggaucgagucagcaccaaaccgguuucugaagaaaauaugcagcauuugaaauaugaagcagcaaacaugaugaagaaaauugaacaacucgaagcuucuaaacguaaacucuugggagaaggcauaggaacaugcucaaucgaggagcugcaacagauugagcaacagcuugagaaaagugucaaauguauucgagcaagaaagacucaaguguuuaaggaacaaauugagcagcucaagcaaaaggagaaagcucuagcugcagaaaacgagaagcucucugaaaaguggggaucucaugaaagcgaaguuuggucaaauaagaaucaagaaaguacuggaagaggugaugaagagaguagcccaaguucugaaguagagacgcaauuguucauuggguuaccuuguucuucaagaaagugaucuccacucaacaaaaaaaacaaaaaaacaaacuaaguaaaaacagguuuagcaguacugagaguguauaaggacaaguaacaaaacucuaaagcugcuucucucuuguugucuuauuucucucucacucucucucucucagucacuucuuuaaugauucugaacugcuuguguuauguaucuauguucuuacacuugccucucuagaauguaauuuugauuauguuuuguuuucauguaucaauuuagcuuucuucccucaaaaucauaaaaucaggaauaucuguacuauau

SEQ ID NO: 36 - DNA, 1431 bp, Artificial Sequence
  Synthetic
tgaggtttcaagcgtccatcatatttgcgacacatatgactgactatttagctcctccctctttctttctcttattttattatctttctccaagaaataaaatagaaaagaaaatatatatggtttcacaaacaccattaccataactacaacgagaagaggatcttttttaaggagaaaagcagagagagaagagacgagtgtgtgaagtttttttgtcttttgtttcttttattacacacaaatagatgaaacgaggaaagctacttcttttgctacttccataaaaaggttcttcctttcgcagagaatcaactttgatcatcttcttccttctctttctttcttcttctccctccagtaatgcttatatagtctcctcctatatctctacctatacatacacaaaccctttatcctcgaaagcttcctcctggttaggtttttatcaaacccttttagccaatcgggaaattaactaaagaagaagatatggtgaggggcaaaactcagatgaagagaatagagaatgcaacaagcagacaagtgactttctccaaaagaaggaatggtttgttgaagaaagcctttgagctctcagtgctttgtgatgctgaagtttctcttatcatcttctctcctaaaggcaaactttatgaattcgccagctccaatatgcaagataccatagatcgttatctgaggcatactaaggatcgagtcagcaccaaaccggtttctgaagaaaatatgcagcatttgaaatatgaagcagcaaacatgatgaagaaaattgaacaactcgaagcttctaaacgtaaactcttgggagaaggcataggaacatgctcaatcgaggagctgcaacagattgagcaacagcttgagaaaagtgtcaaatgtattcgagcaagaaagactcaagtgtttaaggaacaaattgagcagctcaagcaaaaggagaaagctctagctgcagaaaacgagaagctctctgaaaagtggggatctcatgaaagcgaagtttggtcaaataagaatcaagaaagtactggaagaggtgatgaagagagtagcccaagttctgaagtagagacgcaattgttcattgggttaccttgttcttcaagaaagtgatctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgttatgtatctatgttcttacacttgcctctctagaatgtaattttgattatgttttgttttcatgtatcaatttagctttcttccctcaaaatcataaaatcaggaatatctgtactatat

SEQ ID NO: 37 - DNA, 294 bp, Artificial Sequence
  Synthetic
tctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgttatgtatctatgttcttacacttgcctctctagaatgtaattttgattatgttttgttttcatgtatcaatttagctttcttccctcaaaatcataaaatcaggaatatctgtactatat

SEQ ID NO: 38 - DNA, 4915 bp, Artificial Sequence
  Synthetic
atggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggtatgtatcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 39 - DNA, 11960 bp, Artificial Sequence
  Synthetic
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttcatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggtatgtatcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 40 - DNA, 11048 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagggatcctccggacctaggggaggatctggaggaggatctggaggagcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggcatccacggggtgccagctgctgattataaggacgatgacgataaataatctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 41 - DNA, 15286 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccgggtcagagggaaatatgaagaagtcggttgtgacacaagtaagcattggtggatttggggaatccactactgcaaaacaactcacagattaccttgaagatgaagtaggaattgtttggcgttgcagattaaaaacttcgtggactcctcctgggtcttatcctaattttgaaatcgcagacacttcaaatatccccagtattgatgaatataagaaagtggagcctcatgcctttgtccatttcgcagtttttgaatcagctggtcgtgccatggatgctgcagggcaatgcaacctcatcttggatggccagccgttaaaagtcagtttgggtcctaagaatccatattcccttaatcaaagaagaagaacgactgtaccttataagttggctggtattacacttgagattgggacattggtttctcgggatgatttttttgtttcttggagagctgaaggggttgatttcctggtggatccttttgacaacacgtgcaagttttgtttcagaaagagcactgccttctctttcaaggatgcagtgatgcatgctgtgatcaattgtgattataagttggagttattggtgagagatatacaaacagtcaggcagtataaaaccttgcatggctttgtgcttattttgcagctggcttcttcaccccgtgtctggtacagaactgctgatgatgatatttacgatactgtccctggcgatctcttggatgatgatgatccttggatccgtaccactgattttactcaagttggggcaattggtcgatgtcattcatatcgagtgctcatatctccgcggtatgaaaataaactgagaacagccttagattattttaggatgcggagggtgcaagaggaacgtgtgaggtggcctccccgtatccgtaatgagccttgttttggggagcctgtgtcagatcatttcttctgcattcatcacaaggaaggaatctcttttgagatcatgtttctagtaaattcggtattgcacaggggtgtctttaaccagtttcagttgactgagcgattctttgatcttctaagaaaccaacccaaggacgtcaatatagcttctctcaagcatctctgtacctataaacgaccagtttttgatgcgtacaagaggttgaagcttgttcaggaatggattcagaaaaatccaaagcttttagggagtcatgaacaatctgaggatatctctgagatcagaagactagtaattaccccaaccagagcctattgcctacccccagaagttgagctctccaacagggtactcaggagatacaaagctgttgctgaaagatttttgcgggtaactttcatggatgaaagtatgcagaccataaattcgaatgttctctcttactttgttgctcctattgtgaaggatttgacatcaagttctttctcccagaagacctacgtttttaaaagagtgaagagcatattaaccgatgggtttaaactatgtggtagaaaatacagttttctagcattctcagccaatcaactgagagaccgctctgcatggttctttgctgaagacgggaaaacacgtgtgtcagatataaaaacatggatggggaagttcaaagacaagaatgtggcaaaatgtgctgctaggatgggcctgtgcttctcctccacatatgccactgtagatgtcatgcctcacgaggttgacactgaggttccagatattgagagaaatgggtatgttttctctgacggaattggtacaatcacacctgacctcgctgacgaagtaatggagaaacttaagttggatgtgcactacagcccttgtgcttatcagatacgttacgcaggtttcaaaggggttgttgctcgttggccatcaaaaagtgatggaatcaggctagcccttcgagacagtatgaagaagttcttttccaaacatacgatcttggagatctgttcctggacgaggtttcaacctgggttcttaaatcggcagataattacccttctatccgtactaggtgttccggatgaaatattctgggatatgcaggaatccatgctctataaactgaaccgcatccttgatgatacagatgtggcatttgaagttctcacggcatcatgtgctgaacagggaaacactgcagctatcatgcttagtgcaggtttcaaaccaaaaaccgagccgcatctacgcgggatgttgtcttcagtcagaattgcacaactctggggtctcagagaaaaatctcgtatttttgttacttcaggaaggtggctaatgggttgcctagacgaagcagggatacttgaacatggccaatgctttattcaagtctctaaaccgtctatagaaaattgtttctccaaacatgggtctcgttttaaggagacaaagacagatctggaagtagttaaaggctatgtagccattgctaagaatccttgtcttcacccaggggatgtaaggattttagaagctgttgatgtaccccagctgcatcacatgtatgactgccttattttccctcagaaaggtgataggccgcatacaaacgaagcttctggcagtgaccttgacggggacctgtactttgtggcttgggatcagaaactcatccctcccaacaggaaaagctatccggccatgcattatgatgcagctgaagagaagagtttaggccgtgctgtcaaccaccaggtcaatatctacttccccactttttttctgttagctgagatgatgacgcttaagataaaatggacaagtgattccagtataactctctgatatttctggaagtggtggaaactttgtttttggatgcttgtagtgattacttttgcttgcttctgagttcatagattgcgtggcataggttgttaacagcatactgtgttcatcctgtaggagctaagatgatgacatttaagaaaactttgtcatatattgttgattctgatttgtatttttcgcatgccttactgtgactgtgactgtttagttactatcaagggcagtatcatgatgctagtagcttctgccaatgtagttatgaattctttgttattttttcttgctgtgcttgccaagtcttgggtaattgattgcttttgttacaggacataatcgatttctttgcaagaaacttggcgaatgagcagttgggcacaatttgcaatgcacacgtcgttcatgctgatagaagtgagtatggagccatggacgaagaatgtttgctactggcagaactagctgccactgcagttgatttcccaaagacagggaaaattgtgtcaatgcccttccacctaaaaccaaaactctacccagatttcatgggaaaagaagactaccaaacttacaagtcgaacaaaatcttgggtcggctttacagacgggtaaaagaggtttatgatgaagatgcagaagcttcctcagaagaaagcacagacccaagtgccatcccttatgacgctgttcttgaaataccgggatttgaagatttgatccctgaggcatggggtcacaaatgtttgtacgacgggcaactcattggtcttcttgggcaatacaaggtgcagaaagaggaagagattgtgacgggtcacatctggtccatgcccaaatacacaagcaagaaacaaggcgaactgaaagaaagactgaagcattcttataattcccttaaaaaggagttcaggaaagtatttgaggaaacaatccctgaccatgaaaatctcagcgaggaggaaaaaaacatcttgtatgagaagaaggcttcagcttggtatcatgtaacttaccatcccgagtgggtgaagaagtctttagagctgcaagatccagatgagtccagtcatgcggcgatgctgagttttgcttggattgcagctgattatcttgcaagaatcaaaatccggtcacgggaaatgggaagtatcgactcagccaagcctgttgattctttggctaagtttcttgctcagcgtctctaaaaggttatgtttaatgccgtaaggccactctgccatgtatctgtggatttttttgtgtgacgaaacatcttgtctttgcaaaaactgcttgctgttcaggatttgtatagctcaactgcttgccattgtttgtgtaatgtattctactatattatcgtcaaatatgctcttattattttcaaaacaaatttgatttgctcttattatacttgatgttggttaactggtttgatcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 42 - DNA, 11441 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggagcgatcattaaaccaaaccgagatctcaaaccggacggtgtcacagtcgtgactcgtgaagtctgggcggagaatctcgaatcagaatttgaactaatcagcgaaatcatcgacgattatccatttatctcaatggacacagaatttcccggcgtgattttcaaatccgatctccggtttacgaatcccgacgatctttatactcttcttaaagctaacgtcgacgctctcagcttaatccaagtcggtctcactctctctgatgttaacggtaacctcccagacctcggtgacgatcttcaccgaggattcatctgggagtttaatttccgtgacttcgacgtggcgcgtgacgcacacgcgcctgattcgatcgagttgcttcgtcggcaaggtatcgattttgagcggaattgtcgcgacggtgttgagtcggagaggtttgcggagctgatgatgtcgtcggggcttgtttgtaatgaggaagtgagctgggtgacgtttcatagcgcgtatgatttcggttacttgatgaagattctcacgcgccgagaattgcccggcgcgttgggggagtttaagcgcgtgatgagagtgttgtttggggagcgtgtgtatgatgtgaagcatatgatgaagttttgtgaaaggagattgtttggtggtttggaccgggttgcaaggacgcttgaggttaaccgggcggttgggaaatgtcatcaagctggttcggatagtttgcttacgtggcatgcgtttcagaggatgagggatttgtattttgttcaagatgggccggagaaacatgctggggttttgtatgggcttgaggttttttaatctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 43 - DNA, 4908 bp, Artificial Sequence
  Synthetic
atggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 44 - DNA, 11953 bp, Artificial Sequence
  Synthetic
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttcatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 45 - DNA, 5016 bp, Artificial Sequence
  Synthetic
atggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggctgcttgtgttatgtatctatgttcttctgcttgtgttatgtatctatgttcttctgcttgtgttatgtatctatgttcttctgcttgtgttatgtatctatgttcttcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 46 - DNA, 12061 bp, Artificial Sequence
  Synthetic
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttcatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggctgcttgtgttatgtatctatgttcttctgcttgtgttatgtatctatgttcttctgcttgtgttatgtatctatgttcttctgcttgtgttatgtatctatgttcttcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 47 - DNA, 5202 bp, Artificial Sequence
  Synthetic
atggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggtctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgttatgtatctatgttcttacacttgcctctctagaatgtaattttgattatgttttgttttcatgtatcaatttagctttcttccctcaaaatcataaaatcaggaatatctgtactatatcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 48 - DNA, 12247 bp, Artificial Sequence
  Synthetic
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttcatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggtctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgttatgtatctatgttcttacacttgcctctctagaatgtaattttgattatgttttgttttcatgtatcaatttagctttcttccctcaaaatcataaaatcaggaatatctgtactatatcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 49 - DNA, 5195 bp, Artificial Sequence
  Synthetic
atggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggtctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgtctatgttcttacacttgcctctctagaatgtaattttgattatgttttgttttcatgtatcaatttagctttcttccctcaaaatcataaaatcaggaatatctgtactatatcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattg

SEQ ID NO: 50 - DNA, 12240 bp, Artificial Sequence
  Synthetic
cgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcttcatggccgaagatacggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataccatgttagagagcgctagttcgtgagtagatatattactcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaatcaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagccggattcgagatcacaagtttgtacaaaaaagcaggcttcatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagtgaaggtctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgtctatgttcttacacttgcctctctagaatgtaattttgattatgttttgttttcatgtatcaatttagctttcttccctcaaaatcataaaatcaggaatatctgtactatatcctgctcagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaaggtgcgtgacggcgaaatcgaatagactctacatttgctcgtattcgagtttcctttgctaacctttttcatgtttcttcgtttcagttcaacgttgcgaatccaactactggatgccagaagaagctcgagatcgacgatgaccagaaactgtaagattttgcttctctgtttcatcttcattcatttttaatagatatttcgtgctaggttgccagatctattgattggtgtttgtatatatcactgttaaattgataggtctagtaaacagtaattgtcaatttttctatgttcgtcttataagcttaataagagttagaatcttcttctatgtttaatacttacattaaaataagtttcgtatctgctcataaacatatctggattatattattcagcttgatttatgttgatgcagacgtgcgttttacgacaagagaatctctcaagaagtcagtggagatgctttgggcgaggtacagattccatttttcttactttgatctaatgagtgtgaagattagcatttcgaagcagatgatgatgttttgtttatgaattgttgttattgcaggagttcaaaggatacgttttcaagatcaagggtggttgcgataagcaaggtttcccaatgaagcagggagttttgactccaggccgtgttcgccttttgcttcaccgaggtaactaaacataataccttttcttacacttttgcatctataatggatttgtatttgtgtgtgtgtgtgttttgctgtagattaacattgtttcgtaatgggtgaacaggaactccttgcttcagaggacatggaaggagaactggtgagaggagaagaaagtctgttcgtggttgcattgtgagccctgatctctctgttctgaaccttgtcattgtgaagaagggtgagaacgatcttcctgggcttaccgatactgagaagccaagaatgagaggaccaaagagagcctccaagatccgtaaactgtttaacctcaagaaggaagatgatgtcaggacctatgtcaacacttaccgccgcaagttcacaaacaagaagggtgagacactgaacttgagaaatagtggtttctgaatcacacttgagaaataatgttttctgaaacttgtttcttctgtttggatttttgacaggcaaggaagttagcaaagcccctaagatccagaggcttgtgaccccattgactcttcagaggaagagagctagaattgctgacaagaagaagaaaattgctaaggctaattctgatgctgctgattaccagaagcttctcgcctcgaggttgaaggaacagcgtgacaggaggagtgagagtttggcaaagaagaggtcgagactctcttctgctgctgccaagccctctgtcacagcttaaaaaagcttgagaatcaaatcaaagatgtcacctttctagttttgttctcttttcttctccggctattaaaaagttagttcgagttcatattcagctgtttttgttccaagacatgggtacacaatcgctttgggcttgtttcatgatttatgcttttagttgaagaccattttaaactcaatttttattgcttatctttctcagtttttgatcaaagcgtttctccattgaaaaccattttaatcacctgtttcaatcatacataatgttaaacacatgtactggcgattacaataggtatatattttgactgaaatttatggagcattgtatgacggtttgataaatttctgagaaaactcattcttaagttttaagattatctcaacaactggtgaatcaaaactgtgatgtgtggtttgggccaaacgacccataaacgcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcag

SEQ ID NO: 51 - protein, 5 aa, Artificial Sequence
  Synthetic
GGGGS

SEQ ID NO: 52 - protein, 5 aa, Artificial Sequence
  Synthetic
EAAAK

SEQ ID NO: 53 - protein, 7 aa, Artificial Sequence
  Synthetic
AEAAAKA

SEQ ID NO: 54 - protein, 7 aa, Artificial Sequence
  Synthetic
PAPAPAP

SEQ ID NO: 55 - protein, 8 aa, Artificial Sequence
  Synthetic
GIHGVPAA

SEQ ID NO: 56 - DNA, 23 bp, Artificial Sequence
  Synthetic
aaagttggagctgtcattgatgg

SEQ ID NO: 57 - DNA, 23 bp, Artificial Sequence
  Synthetic
aacacgagattctcctctctggc

SEQ ID NO: 58 - DNA, 21 bp, Artificial Sequence
  Synthetic
tggggatctcatgaaagcgaa

SEQ ID NO: 59 - DNA, 22 bp, Artificial Sequence
  Synthetic
acccaatgaacaattgcgtctc

SEQ ID NO: 60 - DNA, 20 bp, Artificial Sequence
  Synthetic
agacgtcgctttgggacttg

SEQ ID NO: 61 - DNA, 21 bp, Artificial Sequence
  Synthetic
atgcgactggttttgaagtgg

SEQ ID NO: 62 - DNA, 22 bp, Artificial Sequence
  Synthetic
gttttgagaacaccattgggat

SEQ ID NO: 63 - DNA, 21 bp, Artificial Sequence
  Synthetic
tttggtgctgactcgatcctt

SEQ ID NO: 64 - DNA, 23 bp, Artificial Sequence
  Synthetic
cgaatttcgaattgtggatgtct

SEQ ID NO: 65 - DNA, 20 bp, Artificial Sequence
  Synthetic
tgctggagtacagatcgcag

SEQ ID NO: 66 - DNA, 32 bp, Artificial Sequence
  Synthetic
  misc_feature (32)..(32): n is a, c, g, or t
gcgagctccgcggccgcgttttttttttttvn

SEQ ID NO: 67 - DNA, 30 bp, Artificial Sequence
  Synthetic
gcgagctccgcggccgcgtttttttttttt

SEQ ID NO: 68 - DNA, 24 bp, Artificial Sequence
  Synthetic
caagtaacaaaactctaaagctgc

SEQ ID NO: 69 - DNA, 25 bp, Artificial Sequence
  Synthetic
tctatgttcttacacttgcctctct

SEQ ID NO: 70 - DNA, 32 bp, Artificial Sequence
  Synthetic
taggaaggaagttcgaagatcaggatattctt

SEQ ID NO: 71 - DNA, 41 bp, Artificial Sequence
  Synthetic
tataatccatctcgagcctgttaatcagaaaaactcagatt

SEQ ID NO: 72 - DNA, 30 bp, Artificial Sequence
  Synthetic
atctggaggaggatccaaggtgcgtgacgg

SEQ ID NO: 73 - DNA, 37 bp, Artificial Sequence
  Synthetic
actctctagacctaggcgtttatgggtcgtttggccc

SEQ ID NO: 74 - DNA, 27 bp, Artificial Sequence
  Synthetic
tcatatttgttcatcttttggtgtgcc

SEQ ID NO: 75 - DNA, 23 bp, Artificial Sequence
  Synthetic
atgctgaccaagaggacttaggc

SEQ ID NO: 76 - DNA, 22 bp, Artificial Sequence
  Synthetic
ggaacttacatgccttactcgc

SEQ ID NO: 77 - DNA, 21 bp, Artificial Sequence
  Synthetic
tccatttctccaacctcaggg

SEQ ID NO: 78 - DNA, 21 bp, Artificial Sequence
  Synthetic
gattgctagtgctgctaacgc

SEQ ID NO: 79 - DNA, 22 bp, Artificial Sequence
  Synthetic
acaccaactatttcgcgtttcg

SEQ ID NO: 80 - DNA, 22 bp, Artificial Sequence
  Synthetic
taaaggcatccacggggtgcca

SEQ ID NO: 81 - DNA, 23 bp, Artificial Sequence
  Synthetic
ttgtccgacgaaaagctttacgc

SEQ ID NO: 82 - DNA, 13581 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccagttctagggctggtccaatgtctaaggaaaagaacgttcagggtggttataggcctgaggttgaacagttggttcaaggtttggcagggacgagactggcttcttcacaagatgatggaggagagtgggaggtcatttccaagaagaacaagaacaaaccaggaaacacttctggaaaaacttgggtttctcagaattcgaatcctcctagagcttggggtggtcagcagcaagggagaggtagcaacgtatctgggagaggaaacaatgtatccgggagaggtaacggcaatggtcggggcattcaagctaacatatctggtcggggacgagcgttgagcagaaagtatgataacaactttgtggcacccccacctgtatctcgccctcctttggaaggaggatggaattggcaggcaagaggaggttctgctcagcacacagctgtgcaggagtttcctgacgtggaggatgatgtggataatgcttctgaggaagagaatgattccgatgctttggatgattctgatgacgaccttgcaagtgatgattatgactcggatgtgagtcaaaagagccatggatcacgaaagcagaataagtggttcaaaaagttctttggcagcttggatagcttgtcgatcgagcagataaatgaaccacagaggcagtggcattgtccagcttgtcagaacggacctggtgccatcgattggtataacctgcaccctctactagctcatgcgaggacaaaaggagctaggcgagttaagctccatagagaattggctgaagttttagaaaaggatctacagatgagaggcgcatctgtcattccttgtggtgagatttatgggcagtggaagggtttgggtgaggatgaaaaggattatgaaattgtctggcctccaatggtcatcatcatgaatactagactggataaggacgataacgataaggtggaattcttctgtcttttacttctttaatttttctcttgcattctactgatcttagaatgttacattgtagtggctcggcatgggcaaccaagagctgctggaatacttcgacaagtatgaggctcttagagcacgccattcctatggtccacagggccatcgtgggatgagtgttctgatgtttgagagcagtgccactggctatttggaggccgaacgcctccaccgggagttagctgagatggggttagatagaattgcctggggtcagaagcgcagtatgttttctggaggtgttcgccaactgtatggcttccttgcaacgaagcaagatctggacatattcaatcaacactctcaaggttctctcccccaaagaaatttgatatatgcttttagttttgtcattggaatttaaagttttgttggtccgtgttaatgcatctgttatgtatatatctatgattcattaggcaaaacaaggctgaaattcgagttgaaatcataccaagagatggttgtaaaggagctgaggcagatctctgaggacaatcagcagctgaactactttaagaacaagctctcaaaacagaacaagcacgccaaggtgcttgaggaatctctggaaattatgagcgagaagctgcgtagaactgcagaggataatcggatcgtgagacagagaactaagatgcagcatgaacagaacagggaagaggtatgatttttcctagaaaatcacaaacttgacattttgtattacctactgattcacatttttgattatattgtccaacaaaaaacctgtggtggtttgaagatggatgcacacgacaggtttttcatggattcaatcaaacagatccatgaaagaagagacgcaaaggaggagaatttcgagatgttgcagcagcaggaacgtgccaaggttgttggccagcagcagcagaacattaatccctctagcaatgacgattgccgaaagaggtatatgtactaactaacataatccctctggcgtttttgtttttcaaacctaagagtaactgaattattccggttttgattctttcgcagagctgaggaagtgtcaagcttcatcgagtttcaagagaaagagatggaggagtttgtggaagagagggagatgctgataaaagatcaagagaagaagatggaagacatgaagaagaggcatcacgaggagatatttgatctggagaaagaatttgatgaggctttggaacagctcatgtacaagcatggccttcacaatgaagatgattgagacaaaagtctggtacacaagacaagactaagtttctttgttttgcttttggtatgtcggaaagtaggagatctgagagactccatttaaatactaggacaaatctaaggagattatagattattatcctccaatttttagtagacggatctaaggaagcattaagttcttgtgactaaaaccaagtttccttagtattttgtttttttttggtaaaatttcatatgaaagttagacatattaccaaacgtcagagtgaatcacagaatggcaaatcaaaatcatgtcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 83 - DNA, 14044 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatcctcaaagattgcaagctcttcgaatagtgatcctgattcaagaagcttacaagtgcttctcgatacttttggttctcgtttttctcttgatgacattgccgctgcttactgccaagctagccagaatgtcgatgtagctggtgagattctctctgcaatggctgaaaagacgcctcaatgtgaccaagttgaaatgaagacagcaacctcaaaaccaactcaggtctatgttcctaaggaagttccacgacaagaggatagcaaagcgaaagtatggaggccgaagagaaattctatatcaaatggtactgtttcaagtgttattggtaaggagtatgctagaaccaggcctatatcaaatgctcccaaagaagcaactaaaccattgaagataaactcaagggatattccagaaactgagatatggagtgaagaaatgcgaaaaagtaacgagggaaagataagcagggctccaactgatgttgaggaatttattgttaagatgcttggagaaggttttcaagctagccaagaagtcattcaccaggttcttggtaagcattctctagctttccttgattctgctttacatatttttcttctttaatggtggtattggcgaccttagctgctaaacttaatctgcttttccactaaactatcaggcgtatgcggatatgatgtgaagaaggtaacccgatgcctttagctctgaaaaataatgtattttgtttctttaacagcgttttgctttcccatatgccttcattttctttctttttctaaaatattttaaatgttttactttttcctggctgagttatattatatagccccatacgttgaagctcatcaagatgaccaccgcttaattatgacctgtgacctttatagttgcatcactctttatttgagctactttaacaataattacaattatacccttgagccactttgacaatccttttctctgtcttggtactactgctgactaaaatttgagataaagttgacttttcataccgactgtcgcctctaattttgatcacttgattgttagtggtatctctataactcttggcatatggctcttgtgagtatttcatttgatcctgcttttgttacaacttcttcttttggcatctacactatattcttcattatattgcagagtacggagaaactgcttgacttttctgataccaagaaatatgctgatgttggaatttccaatgaagttgtgagcttttattatgactttcagtttggtctttgttatagaatagcgtcttcttctaaatctgactctgttgctttgtttccctttctatatagatgtcaaaagttgatcctcaaagacaatctacatcctgcaaccaaatggaactacaaaaaatttctcagaggcacgtttttcctagtcaatctttgtaccgaaactttccctttgcaatccttttgattttttttcctcttcaaattgctttttgtgatgatgcagtgttggagcaagaacctttacaggttcacaggaagggcgtaatgctaagaatggtcttgaaaaggaagttttagaagctttgttctcaggtgcggaaagatatgtggacaaaccaaaagtaactcgacattttggagaaaggagacctggagttgctggtcgtcctgtttttaaacctctggaagacccttttcaagagagagtagttgctgtgaaacaatcgtccaatacatctaaagaaggtaaccatgccgtagtcttacatacacgtttatttgagtttattcttttgttcctcaagcgtttgctcaactatcatttttctcctgtgatagatgacgatgaaaatgaatttaaggcacatcgtaaagcagtacgcgagcatttgaatcagatgaaagaatattatggagctgtaagtctttgagataattgcccgatatacctttatatgtttgtaatggctatatgattgtcatgatgtgatggttgcataactaaaacctcttcctttttgacaggctgcagaagcattctccaaaggagaaaccgagcgagcacacagactcgtggagaaggtacaatttggaaagaaatttttggttgaaagtgatcagtgtctttcttgtcttctatgtaacaactatatgatatcttccatgaaatatagggacacttttttggacaaaaagctcgagaagcagatgacaaatctgtagcaaagatgattgacgtcaagtcagtgaatttctcaatccatttattatcagtcctctattgtcattttcttctgtatgctccattggcaggaagagttgaatttttttttaaaccattcttatagtcaagaagatgattcaacttacgaggaagacgaggtagtgacagtaaatatgaatgaacacgaagcaaaagaagctcttcgtcttctcaagcgccaacttatttacttctctggcatttcatgtaaagttttaaatccctctttttaaaccttttttgttctatcagaaacaaatctaatctgtttgatgagtttgtattctatttgtgatttagctttcaagtacctcagagtccaattgggtgagaagaaggaagatttcaagagcaaacgaaaggtcccatctttactcatctgttttcatctcttcaaagaacttgaagtcttcatctctgaacagagcttgaaattgcttctatttatattgttttcttagcatatcgtaaagctgctggagggagaatccattccttggactgaagaagatggcggcctcgtgataatgattcgggttgatgagattgaccccaagaaattgagttttgccaagaaataagaaccattggtttattcttttgcttcattctattagaaattggttgcaacgtttaaagcattatgatttgttatgtaaccaaacaaagatgcattttgcggaaatatgatttttggtttcggaatcgaagaattatcaagccatctgacattacaatatgccctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 84 - DNA, 14682 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccagtgtgagtggatacaaatcggatgacgaatattctgtaatcgcagacaagggagagattggatttatagactatcagaatgatggatcttctggttgctacaatccatttgatgaaggtccagttgttgtttctgttcctttcccatttaagaaagagaagcctcaatctgtaactgttggagagacttcttttgattccttcactgtcaagaacactatggatgagcctgttgatctctggactaagatttacgcatctaaccctgaggattcttttactctctcgatattgaaaccgccttcgaaagattcagacttgaaagagagacagtgtttttatgaaacttttactctggaagataggatgcttgagcctggtgacactttgaccatttgggtgtcttgtaagcctaaggacattggtttacataccactgttgttactgttgactggggaagtgatagggttgagcgggttgttttccttttggctgaggataagatttcgagttctctgacttctaacaggccttactctagaagtagaagggctccgaagaaagattttgctgtggatgattatgtgaagggatcacgcccttcaaaggtggttgaacgtagttttagaaataggcttcctctgtatgaaattccaaaggagattagagagatgatcgagaataaggaattccctgatgatttgaacgaaggtcttacagcaaggaactatgctaactactataaaactttactgattatggaagaattacaactcgaggtatcctcattgtttcagatttttgttaataactcaataaacctcttgtaccatgaatcatgtttcttgtaattttttggttgtttaggaagacatgcgagcttatgatatggagaatgtttcaatgaagagaaggggtatctatttgtccctcgaagttcctggccttgctgagagaagaccttcccttgtccatggagacttcatctttgttagacatgcatatgatgatggaactgaccacgcttatcaggttttgttctcgctttactttctcatactttcagatcactttctgcttcacaactgcaaatatgtttaatcatttatcttttagaggatacaaaattgaaatcaatttctcacagggctttgtacaccgcgtagaggcggatgaagtacatatgaagtttgcctctgagttccaccaacgccacacagctgggagtgtctataatgtgaggttcacatacaatcgaatcaacactagaaggttgtatcaggctgttgatgcggcagaaatgttggatccaaacttccttttcccatccttgcactctgggaaaaggatgataaagacaaaaccctttgtgcccatttcaccagctcttaatgcagagcagatttgttccattgaaatggttcttggctgcaaaggagcaccaccatatgtaatccatggacctcctggtactgggaaaaccatgacattggtggaggctatagttcaactttatacaacacagagaaatgctcgggttcttgtctgtgctccctccaatagtgcagctgatcacattttagagaaactcctttgtttagagggagttcgcatcaaggacaatgagattttcaggctgaatgcagctactcgttcatatgaagaaattaagcctgagattatccgcttttgcttctttgatgagttaattttcaagtgcccccctctcaaagctttgactcgttacaaactcgttgtgtcaacatatatgagtgcatcgcttcttaacgcggaaggtgtaaaccgtggtcacttcactcatattctcttagatgaggctggtcaagcttcagagcctgaaaacatgattgctgtatcaaatctctgcttgacagaaactgtggttgtgcttgctggagatccaaggcaattgggtccggttatatactcaagagatgctgagtcactcggcttggggaagtcatacttggaaagattgtttgagtgtgattattactgcgaaggggatgagaattatgtaacaaagctagtcaagaactacagatgccacccagagattcttgatctcccgtcaaagctcttctatgatggagaactggtagcttccaaagaagacacagactctgttcttgcctcgttaaacttccttccaaacaaagaatttccgatggttttctatgggatccaaggatgcgatgagagggaaggtaacaatccgtcctggttcaaccgtatagagatcagcaaagttatagagacaatcaagagattaacagcgaatgactgtgtgcaagaagaggatatcggagttataactccttacaggcagcaagtaatgaagatcaaggaggttcttgacaggttagatatgactgaagtcaaagttggtagtgtagagcaattccaaggtcaagagaaacaggtcatcatcatttcaactgtaagatctactatcaaacacaacgagttcgatagagcctattgtctgggcttcctgagcaatccaaggaggtttaatgtcgccataacccgtgcaatctctctgctagtgatcatcggaaacccacatatcatatgcaaggtaccacttacttagttgttctggataaagacataatgcgttgacatcattcccaatctctcaactaataaaacgtattatatgaaacaggacatgaactggaacaagcttttgtggcgatgtgtggataacaatgcttaccaaggatgcggtttaccagagcaggaggagtttgtggaggaaccattcaagcaagaaggaagcagcaatgggcctcaataccctccagaagctgagtggaacaacagtggagagctcaacaatggtggtgcgaatgagaatggtgaatggtctgatgggtggaacaacaatggcggcacaaaggaaaaaaatgaatggtctgatggatggaacagcaatggtggtgggacaaagaagaaagatgaatggtctgatgggtgggacaacaatggcggcacaaacgggatcaatcaagaaggaagcagcaacgctccccaagaccctcaagaagccgagtggaacgatagtggcgaggtcaagaatggtggcacaaaggagaaagatgtcaggtctgatgggtggaacaacaatggcggaaaaaatgaaaaagaagaatgttgtgatggttggaaagacggcggcagtggtgaagagatcaagaatggtggtaagtttgaaaccagaggtgattttgtagccaaggaagaagatgagtggtctgatgggtggaagtaaattaaaaaaaaagtagagtaataagggttaattgtaggtggatggacaaaaggctattttggatttgttcatattttctttctatctctcaacctttggtttttgcagagtctcgtttttaaatgcatgatttggtgtgtataaaattaaggaaactgttttattaatagggctatcaaatacaacaaaactctaaaacattattttaacaagaatcaggtaaaatatgaacgtaaagaacgtcttttatagtatggtattgctctttaattagatgaatttgataatcgtaaaggcgaaagcaaatagaagatacgaaagcttgagagagtgcgaagagcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 85 - DNA, 16219 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaacaatggctgcgatgaagtctctccgcggtcagagatcgctcttctgggtagtgttgaaacaatgttggagaaaatctacgagaaacacaatcatcgtcttccgataagtgtggagacaaggcgaaaactttcttcgatttctgaggaattggctctggaaacacttaggaaagttttcaataagccgtatttgaagaccctcgatgggttaatcatgtattttgttaaagggacagtcactgttgatggctctccacgcctgagtcccggcgagtctccagtacagtctcctagaactcctgccaaaaaaagttgcagggcttctcaaggtcagtcagtatctgatgggcttgtgttgttttatatgctctagagttttatttatgagtattggtcaattgttttagcagatgtgtctcttgatttggaaactccctctcctaagttcatgaaaagagaagaaaatggtggatctaagtacattcctccgcttttagctttgggtgaacttgaattcaagaaggcgtttctgttgcttagctatattggagggtatgttaggctggaacaatttttgatctttaataatcagttcttatctattcctttggaaagtgatcgttttttgttacagggaatcccttgttgaggaggttatatctggtgatcagatcagaaaatggaaggatttgccaatggtttcatatgaagcagcggtttggaaccggttgggacagcgttattgttcccccaaggagcgaagaagggtatggcagttctctgttctgttcttcatcatgtactttcttaagtgaattgacaagacatggagagagacctggtggatacttcaaattcctttatcttctgcaatttatgaagaatctgtactaattgcgtcttcttacatgcttctttctccagccacttgaaggggatagtgggatgacccattactatcaatgtcatgttgctaccgatggtagttacaaattcaaggtataacctcctctctaagctctgtctttcattttttgcattttcccgtgttgctacgttctttgtgttagcttgatatatgttctttgttttcaaaggatctgaattcaagagatactactagaagctgttgacataatttgtttgcaagtcgctatcatatatctaataaatgtgtatatcctactattatgataaagtcctggacagtgtcttaatactatttttacttattgataaactgtgttcacagggtcatcttctggagaacactggaacacacctgcacaaggttttgggtgatgataacgttctaactgtcaaatttgataaagtgttaggcgtggaaacctattgtaatgacctttactctacatacaaagggattgctaagaatggtatcatggttgggttacgccgttatcgattttttggtgggtcttttctccttcttaatacctcttattgagcatataacatttttcattttttaggtgctaatgccatgtctagcaacatagcagatcattttcatctttgtgtttaagactgcatattgtagcttgtttacgtttcctttacaaacagttttcaaagatggagggaaagaagagaagaagaaagatgtttctacaaagggagttaaatgctactttatacgcacagactcgacagcttcaattgatatgcaaaatccctatatcttcgccggaaagtcaatgcatgaagcacgtatgcattttatgcatgtgaatactttgtcatctttgcccaactatatgtcaaggtactctattgtaggcatatgaggatattctactgtttcatctctaaactcttgagatttcctcctttgtaatcagagtcaccttttgttgatgcttcaggttttctttaattctgtcaaagactaaaacgctcgaagttgacatgactggaattacctttgagcaaatcgatgatatacactgccatgtacttccttaattcatgttttcttcaggaactgcgtctctttctaatgcctttatttagttgtatttgatatgtgccttggctttttacctctctgacaattgcaggatcaagatgataaggatgttcttgacaagaatgggaaaccttgtatacattcagatggaactggctacatctctgaggaccttgctcggatgtgtccagttaatatttttaaagggaaatctatgagaagtaacaatattcaggtctttcctgtacttccttgattatttgatggtttcttagttgccttttgttcactcttgattccttatcatgtttgtttcttatttgttttcgatcaagagtaagaatttaaattttgaaggacagggaccatgtggccaagagccggtatgtagtttatattccacgtttaagcgctccactcagtatttttctatcacttaaaacaagtagtaataaatcgttttttcaacagcctctgctgatccagtttcggattttttataatggctatgctgttaagggaacttttctcacaaacaagaaagtaagccagctctctttcatgtcacaaaaatcagatttgcaagctattcctcagttttctcctttatttggcagcttcctcctcggactgtccaggttcgaccttctatgatcaaggtgtatgaagatcgaaccttgtcaaatcttagtacctttaactccttggaggtagtcactacaaggtagctgtcacaagagttcttactatcctgaagctgaatttctcgcttgatgttttttcttttctttttttgatctctggttgtttgcatcttctagtaatccaccaagaaaggcgaggctatcaagaaatttggtggcgctacttagttatggaggagtccctaatgacttctttttgaatatattgcgcaacacgctggaagagtccaaaaccatattctacagtgaacgtgctgcgtttaaaggtgctaagaatcttaaagctttcttgtttgtttgcagtttcttactactcctgaagctactgagttaaaatatgctcggaagtttgaggagacactcaatgggtctttttattaagttcatttgtctgtgcagctgccattaattatggggacgaccagtacactgcagatatgatcttggttggtatccctcttgatgaaccatacttgaaggatcgtctttcttatctcttaaagacagagaggaatgctctcaaagcaggaaggtttcctattgacgaatcatactatatcatgggcacagtggatcccaccggggagcttaaggaaaatgaaatctgtgtcatcctgtaggctagcaaattcttctctttctctttcgctttgagttttgcatgtaatcatcaaaaaacagtgattagcttccttggaattatgagttcttccctacaagtagctgctcattttcactgctttcttttcatggttttgtagccattctggccaaatttccggggatgtgttagtttacagaaatcctggattacattttggagacatacatgtacttaaggctacctatgttaaggccttagaagactatgttggaaatgccaagtttgctgtgttcttccctcagaaaggtccaagatccttgggcgatgagattgctggtggtgactttgatggcgatatgtatttcatatccagaaatcccaaggtactctgctatatccatcacatttgaaaataaactcttcattgctgagacctgacctgtgaaccggaattcgtctatttagctactggaacacttcaagccaagtgaaccatgggtgagttcttctaaacctagtaaaatttactgtggaagaaagccaagtgaactatcagaagaagagttggaagaagagctattcaaaatgttcttaaaagcaagattttgtaagaggtacataatttaactgtcggcttactttcaagctcacaatagttcgtttagttaacatatgccttgttttcacggtttacagagacgttatagggatggctgcagattgctggctaggaataatggaccctttcctcaccttaggagatgagagtgctaaggaaaaatatgaaaggaagaagaatattcttaagttgattgatatatactatgatgctcttgatgcaccaaaaaagggggctaaggttgatctcccgcccgatctagagatcaaaaattttccacattacatggagcgtgaccctaagagagatttcaggtccacttctattcttggattaatctttgacactgtggattctcataatgcagaggaacccccaccatctggtacggatcttctagtttctatcaataatcagagtacacaagaactcatcatctcgaatatgtattaacgtctatattgtatgtttcaattcagaaatcagtaaactctggtattttgaagatgaaccggtccctaagtctcacatggataaatttacatcctggtacgagaattaccgaagtgaaatgtcccaagcgatgatggaaactgataaggttaagaggaatcaattaaccaatgaagttatccagaggtacaagcaggtaagctctcgctcaatccaatgcaaagatctgaccaatagatttcatgaatcacattttttgttgattagtccatataatgcttcttaacacaactcatcggttgaacggtttcttctgtctgtatcaggatttctatggcgctgcagggtttgaagacagcaataaaagtctggaagaactctacccccaagccttggcactctacaacgtcgtttacgattatgccattcaggaaggtgttgcgaaatgtacatttgcctggaatgttgcaggaccggtcctgtgcaaattttaccttaagaaaacgaaggataaatcagtagtggcttcaacgtctgtgcttaaaaagcttttgggttgaacagacttacttgtcctgtttgttgtcttcatgtatcataagatgcgttagacaagtatctgacctaggtaaccgaacttataggcgaagctatgtggtgtacttcctttgtttaagttataacttaagatttgtcatctagtgtacaagtaattccgtgtgtatgtttgcttatggaataaataaagaaaactaatgcttatatttaatttcaattaattgtacgtggcactttcagagtccacctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 86 - DNA, 14006 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagaaaatagcaagacatgtttacgttgacaaacaaacaaaaattaagcaagtcatgttcgtagcaataaattaatagtgggaacaattaagttaagcgaaaaaggaaaaaaaaaggtacaaaaatgaaaacaaaatcaaactgaatgaaaatttggagtccagaatcggaaaaacgaggccgttttagagcttaataagcttcctcatttgtctcttcttcgtcagtttattttcttcctccggagtcctgactcactactctcactctccggcgctttaaacttacgttctccgtcgtttactctgtaagttttctgccttagagcctccgatcgcctcaccgcatgcattctgtgctcgatttctctttttcttcgctggaaaaattgccctaatgttctcgatttcgaaggtttttgtgctatgggttacttttttccctatattttatagttcttaggtaacgatacctgcgtcttactgtttttgttcattttgttgtgctttcaccgtttagtcgctgatcggagtatttgactgtgaaaaatccttcgttttttggtttttgtttcatataaatcggattgatctaccttttgtgctttgatgtttgttttttgagcctatgcgttgttggcttgttataacttcacgttcatgtgtggattttgagattttggtagtgactgtgggtttctttggtggctataggttgtaaaaatgagttctagggctggtccaatgtctaaggaaaagaacgttcagggtggttataggcctgaggttgaacagttggttcaaggtttggcagggacgagactggcttcttcacaagatgatggaggagagtgggaggtcatttccaagaagaacaagaacaaaccaggaaacacttctggaaaaacttgggtttctcagaattcgaatcctcctagagcttggggtggtcagcagcaagggagaggtagcaacgtatctgggagaggaaacaatgtatccgggagaggtaacggcaatggtcggggcattcaagctaacatatctggtcggggacgagcgttgagcagaaagtatgataacaactttgtggcacccccacctgtatctcgccctcctttggaaggaggatggaattggcaggcaagaggaggttctgctcagcacacagctgtgcaggagtttcctgacgtggaggatgatgtggataatgcttctgaggaagagaatgattccgatgctttggatgattctgatgacgaccttgcaagtgatgattatgactcggatgtgagtcaaaagagccatggatcacgaaagcagaataagtggttcaaaaagttctttggcagcttggatagcttgtcgatcgagcagataaatgaaccacagaggcagtggcattgtccagcttgtcagaacggacctggtgccatcgattggtataacctgcaccctctactagctcatgcgaggacaaaaggagctaggcgagttaagctccatagagaattggctgaagttttagaaaaggatctacagatgagaggcgcatctgtcattccttgtggtgagatttatgggcagtggaagggtttgggtgaggatgaaaaggattatgaaattgtctggcctccaatggtcatcatcatgaatactagactggataaggacgataacgataaggtggaattcttctgtcttttacttctttaatttttctcttgcattctactgatcttagaatgttacattgtagtggctcggcatgggcaaccaagagctgctggaatacttcgacaagtatgaggctcttagagcacgccattcctatggtccacagggccatcgtgggatgagtgttctgatgtttgagagcagtgccactggctatttggaggccgaacgcctccaccgggagttagctgagatggggttagatagaattgcctggggtcagaagcgcagtatgttttctggaggtgttcgccaactgtatggcttccttgcaacgaagcaagatctggacatattcaatcaacactctcaaggttctctcccccaaagaaatttgatatatgcttttagttttgtcattggaatttaaagttttgttggtccgtgttaatgcatctgttatgtatatatctatgattcattaggcaaaacaaggctgaaattcgagttgaaatcataccaagagatggttgtaaaggagctgaggcagatctctgaggacaatcagcagctgaactactttaagaacaagctctcaaaacagaacaagcacgccaaggtgcttgaggaatctctggaaattatgagcgagaagctgcgtagaactgcagaggataatcggatcgtgagacagagaactaagatgcagcatgaacagaacagggaagaggtatgatttttcctagaaaatcacaaacttgacattttgtattacctactgattcacatttttgattatattgtccaacaaaaaacctgtggtggtttgaagatggatgcacacgacaggtttttcatggattcaatcaaacagatccatgaaagaagagacgcaaaggaggagaatttcgagatgttgcagcagcaggaacgtgccaaggttgttggccagcagcagcagaacattaatccctctagcaatgacgattgccgaaagaggtatatgtactaactaacataatccctctggcgtttttgtttttcaaacctaagagtaactgaattattccggttttgattctttcgcagagctgaggaagtgtcaagcttcatcgagtttcaagagaaagagatggaggagtttgtggaagagagggagatgctgataaaagatcaagagaagaagatggaagacatgaagaagaggcatcacgaggagatatttgatctggagaaagaatttgatgaggctttggaacagctcatgtacaagcatggccttcacaatgaagatgatcctaggggaggatctggaggaggatctggaggagcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggcatccacggggtgccagctgctgattataaggacgatgacgataaataatctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 87 - DNA, 14801 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgaggcaatttggctcttcgattttcaaatttggtttttttataaataacaagaatttgtcactgtcttccctataaaatttggaggaagcaaaggcgcttgcttttcgcagcaaagcctatagaaactttacttccttctcctctcgtctcatgcaccgaatgtatttccctgcttaaccaattctctactttgtcctcttcttctccgtttcgattttacaagattcgatcttttgcaatcgaaatacctaatattccgattctcgattccggcgtcttttgcttcgggcttttttggtaagtgtactcttacactatctccgtctctactcttttgcgtcttgcattgctgattgttggaaatggatttctttgtggtgtaatgcggtttccaaacgagttcgagttggctcttttagaaagattctttcttctttagtctgattctcaaggtttcacattcttcgttttgataatgcgtttccctaattgcgttgtgattatcgataccgagtaaaaaggtgttcttggacttttcactttgccattgatttgcttcacttgtattactttagaactctcagaaacgatcattgattggcttgtgtagagataattgagcagttccatttatctacttagatcggagatataatgcctgccaggtgtttgttatatgcaattttgggtagagcattgaaattttgaccgagagtagtatcatgattacaagccatgttctgctttagtttcgtagtcagtgatggttattaggaagatgattgaaactagctttttttgcagcttcaaatgcataacattttggttgtaatgtagggaacattttaatcattcagcattctacattttgcatagctactgttgttaaccaagccatttgttttgcactgtgaattagtgggaggaatgtcaaagattgcaagctcttcgaatagtgatcctgattcaagaagcttacaagtgcttctcgatacttttggttctcgtttttctcttgatgacattgccgctgcttactgccaagctagccagaatgtcgatgtagctggtgagattctctctgcaatggctgaaaagacgcctcaatgtgaccaagttgaaatgaagacagcaacctcaaaaccaactcaggtctatgttcctaaggaagttccacgacaagaggatagcaaagcgaaagtatggaggccgaagagaaattctatatcaaatggtactgtttcaagtgttattggtaaggagtatgctagaaccaggcctatatcaaatgctcccaaagaagcaactaaaccattgaagataaactcaagggatattccagaaactgagatatggagtgaagaaatgcgaaaaagtaacgagggaaagataagcagggctccaactgatgttgaggaatttattgttaagatgcttggagaaggttttcaagctagccaagaagtcattcaccaggttcttggtaagcattctctagctttccttgattctgctttacatatttttcttctttaatggtggtattggcgaccttagctgctaaacttaatctgcttttccactaaactatcaggcgtatgcggatatgatgtgaagaaggtaacccgatgcctttagctctgaaaaataatgtattttgtttctttaacagcgttttgctttcccatatgccttcattttctttctttttctaaaatattttaaatgttttactttttcctggctgagttatattatatagccccatacgttgaagctcatcaagatgaccaccgcttaattatgacctgtgacctttatagttgcatcactctttatttgagctactttaacaataattacaattatacccttgagccactttgacaatccttttctctgtcttggtactactgctgactaaaatttgagataaagttgacttttcataccgactgtcgcctctaattttgatcacttgattgttagtggtatctctataactcttggcatatggctcttgtgagtatttcatttgatcctgcttttgttacaacttcttcttttggcatctacactatattcttcattatattgcagagtacggagaaactgcttgacttttctgataccaagaaatatgctgatgttggaatttccaatgaagttgtgagcttttattatgactttcagtttggtctttgttatagaatagcgtcttcttctaaatctgactctgttgctttgtttccctttctatatagatgtcaaaagttgatcctcaaagacaatctacatcctgcaaccaaatggaactacaaaaaatttctcagaggcacgtttttcctagtcaatctttgtaccgaaactttccctttgcaatccttttgattttttttcctcttcaaattgctttttgtgatgatgcagtgttggagcaagaacctttacaggttcacaggaagggcgtaatgctaagaatggtcttgaaaaggaagttttagaagctttgttctcaggtgcggaaagatatgtggacaaaccaaaagtaactcgacattttggagaaaggagacctggagttgctggtcgtcctgtttttaaacctctggaagacccttttcaagagagagtagttgctgtgaaacaatcgtccaatacatctaaagaaggtaaccatgccgtagtcttacatacacgtttatttgagtttattcttttgttcctcaagcgtttgctcaactatcatttttctcctgtgatagatgacgatgaaaatgaatttaaggcacatcgtaaagcagtacgcgagcatttgaatcagatgaaagaatattatggagctgtaagtctttgagataattgcccgatatacctttatatgtttgtaatggctatatgattgtcatgatgtgatggttgcataactaaaacctcttcctttttgacaggctgcagaagcattctccaaaggagaaaccgagcgagcacacagactcgtggagaaggtacaatttggaaagaaatttttggttgaaagtgatcagtgtctttcttgtcttctatgtaacaactatatgatatcttccatgaaatatagggacacttttttggacaaaaagctcgagaagcagatgacaaatctgtagcaaagatgattgacgtcaagtcagtgaatttctcaatccatttattatcagtcctctattgtcattttcttctgtatgctccattggcaggaagagttgaatttttttttaaaccattcttatagtcaagaagatgattcaacttacgaggaagacgaggtagtgacagtaaatatgaatgaacacgaagcaaaagaagctcttcgtcttctcaagcgccaacttatttacttctctggcatttcatgtaaagttttaaatccctctttttaaaccttttttgttctatcagaaacaaatctaatctgtttgatgagtttgtattctatttgtgatttagctttcaagtacctcagagtccaattgggtgagaagaaggaagatttcaagagcaaacgaaaggtcccatctttactcatctgttttcatctcttcaaagaacttgaagtcttcatctctgaacagagcttgaaattgcttctatttatattgttttcttagcatatcgtaaagctgctggagggagaatccattccttggactgaagaagatggcggcctcgtgataatgattcgggttgatgagattgaccccaagaaattgagttttgccaagaaacctaggggaggatctggaggaggatctggaggagcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggcatccacggggtgccagctgctgattataaggacgatgacgataaataatctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 88 - DNA, 16276 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagggcgagaaagtagtggcagatttgtaaatatagtagtgatggttgggcattttgataaaaattaaaaaattaataaacggctagaatagcgtactgcatataaatctccgtgtagtcaaatcaaagtatcaaacagatttacacaaaacttctgtttttttctttttttgaaacctaaaggacgaaactttgagaaaatggcagcaaaagcaattccagagttaaacaagtttctctcttcccactagggctatatattatgaaccctagattatagacactgactccgattttggggagaaaaaatgatgaacaatggctgcgatgaagtctctccgcggtcagagatcgctcttctgggtagtgttgaaacaatgttggagaaaatctacgagaaacacaatcatcgtcttccgataagtgtggagacaaggcgaaaactttcttcgatttctgaggaattggctctggaaacacttaggaaagttttcaataagccgtatttgaagaccctcgatgggttaatcatgtattttgttaaagggacagtcactgttgatggctctccacgcctgagtcccggcgagtctccagtacagtctcctagaactcctgccaaaaaaagttgcagggcttctcaaggtcagtcagtatctgatgggcttgtgttgttttatatgctctagagttttatttatgagtattggtcaattgttttagcagatgtgtctcttgatttggaaactccctctcctaagttcatgaaaagagaagaaaatggtggatctaagtacattcctccgcttttagctttgggtgaacttgaattcaagaaggcgtttctgttgcttagctatattggagggtatgttaggctggaacaatttttgatctttaataatcagttcttatctattcctttggaaagtgatcgttttttgttacagggaatcccttgttgaggaggttatatctggtgatcagatcagaaaatggaaggatttgccaatggtttcatatgaagcagcggtttggaaccggttgggacagcgttattgttcccccaaggagcgaagaagggtatggcagttctctgttctgttcttcatcatgtactttcttaagtgaattgacaagacatggagagagacctggtggatacttcaaattcctttatcttctgcaatttatgaagaatctgtactaattgcgtcttcttacatgcttctttctccagccacttgaaggggatagtgggatgacccattactatcaatgtcatgttgctaccgatggtagttacaaattcaaggtataacctcctctctaagctctgtctttcattttttgcattttcccgtgttgctacgttctttgtgttagcttgatatatgttctttgttttcaaaggatctgaattcaagagatactactagaagctgttgacataatttgtttgcaagtcgctatcatatatctaataaatgtgtatatcctactattatgataaagtcctggacagtgtcttaatactatttttacttattgataaactgtgttcacagggtcatcttctggagaacactggaacacacctgcacaaggttttgggtgatgataacgttctaactgtcaaatttgataaagtgttaggcgtggaaacctattgtaatgacctttactctacatacaaagggattgctaagaatggtatcatggttgggttacgccgttatcgattttttggtgggtcttttctccttcttaatacctcttattgagcatataacatttttcattttttaggtgctaatgccatgtctagcaacatagcagatcattttcatctttgtgtttaagactgcatattgtagcttgtttacgtttcctttacaaacagttttcaaagatggagggaaagaagagaagaagaaagatgtttctacaaagggagttaaatgctactttatacgcacagactcgacagcttcaattgatatgcaaaatccctatatcttcgccggaaagtcaatgcatgaagcacgtatgcattttatgcatgtgaatactttgtcatctttgcccaactatatgtcaaggtactctattgtaggcatatgaggatattctactgtttcatctctaaactcttgagatttcctcctttgtaatcagagtcaccttttgttgatgcttcaggttttctttaattctgtcaaagactaaaacgctcgaagttgacatgactggaattacctttgagcaaatcgatgatatacactgccatgtacttccttaattcatgttttcttcaggaactgcgtctctttctaatgcctttatttagttgtatttgatatgtgccttggctttttacctctctgacaattgcaggatcaagatgataaggatgttcttgacaagaatgggaaaccttgtatacattcagatggaactggctacatctctgaggaccttgctcggatgtgtccagttaatatttttaaagggaaatctatgagaagtaacaatattcaggtctttcctgtacttccttgattatttgatggtttcttagttgccttttgttcactcttgattccttatcatgtttgtttcttatttgttttcgatcaagagtaagaatttaaattttgaaggacagggaccatgtggccaagagccggtatgtagtttatattccacgtttaagcgctccactcagtatttttctatcacttaaaacaagtagtaataaatcgttttttcaacagcctctgctgatccagtttcggattttttataatggctatgctgttaagggaacttttctcacaaacaagaaagtaagccagctctctttcatgtcacaaaaatcagatttgcaagctattcctcagttttctcctttatttggcagcttcctcctcggactgtccaggttcgaccttctatgatcaaggtgtatgaagatcgaaccttgtcaaatcttagtacctttaactccttggaggtagtcactacaaggtagctgtcacaagagttcttactatcctgaagctgaatttctcgcttgatgttttttcttttctttttttgatctctggttgtttgcatcttctagtaatccaccaagaaaggcgaggctatcaagaaatttggtggcgctacttagttatggaggagtccctaatgacttctttttgaatatattgcgcaacacgctggaagagtccaaaaccatattctacagtgaacgtgctgcgtttaaaggtgctaagaatcttaaagctttcttgtttgtttgcagtttcttactactcctgaagctactgagttaaaatatgctcggaagtttgaggagacactcaatgggtctttttattaagttcatttgtctgtgcagctgccattaattatggggacgaccagtacactgcagatatgatcttggttggtatccctcttgatgaaccatacttgaaggatcgtctttcttatctcttaaagacagagaggaatgctctcaaagcaggaaggtttcctattgacgaatcatactatatcatgggcacagtggatcccaccggggagcttaaggaaaatgaaatctgtgtcatcctgtaggctagcaaattcttctctttctctttcgctttgagttttgcatgtaatcatcaaaaaacagtgattagcttccttggaattatgagttcttccctacaagtagctgctcattttcactgctttcttttcatggttttgtagccattctggccaaatttccggggatgtgttagtttacagaaatcctggattacattttggagacatacatgtacttaaggctacctatgttaaggccttagaagactatgttggaaatgccaagtttgctgtgttcttccctcagaaaggtccaagatccttgggcgatgagattgctggtggtgactttgatggcgatatgtatttcatatccagaaatcccaaggtactctgctatatccatcacatttgaaaataaactcttcattgctgagacctgacctgtgaaccggaattcgtctatttagctactggaacacttcaagccaagtgaaccatgggtgagttcttctaaacctagtaaaatttactgtggaagaaagccaagtgaactatcagaagaagagttggaagaagagctattcaaaatgttcttaaaagcaagattttgtaagaggtacataatttaactgtcggcttactttcaagctcacaatagttcgtttagttaacatatgccttgttttcacggtttacagagacgttatagggatggctgcagattgctggctaggaataatggaccctttcctcaccttaggagatgagagtgctaaggaaaaatatgaaaggaagaagaatattcttaagttgattgatatatactatgatgctcttgatgcaccaaaaaagggggctaaggttgatctcccgcccgatctagagatcaaaaattttccacattacatggagcgtgaccctaagagagatttcaggtccacttctattcttggattaatctttgacactgtggattctcataatgcagaggaacccccaccatctggtacggatcttctagtttctatcaataatcagagtacacaagaactcatcatctcgaatatgtattaacgtctatattgtatgtttcaattcagaaatcagtaaactctggtattttgaagatgaaccggtccctaagtctcacatggataaatttacatcctggtacgagaattaccgaagtgaaatgtcccaagcgatgatggaaactgataaggttaagaggaatcaattaaccaatgaagttatccagaggtacaagcaggtaagctctcgctcaatccaatgcaaagatctgaccaatagatttcatgaatcacattttttgttgattagtccatataatgcttcttaacacaactcatcggttgaacggtttcttctgtctgtatcaggatttctatggcgctgcagggtttgaagacagcaataaaagtctggaagaactctacccccaagccttggcactctacaacgtcgtttacgattatgccattcaggaaggtgttgcgaaatgtacatttgcctggaatgttgcaggaccggtcctgtgcaaattttaccttaagaaaacgaaggataaatcagtagtggcttcaacgtctgtgcttaaaaagcttttgggtcctaggggaggatctggaggaggatctggaggagcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggcatccacggggtgccagctgctgattataaggacgatgacgataaataatctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 89 - DNA, 16939 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccgtgagaaagagaagaacggatgctccatctgaaggaggtgaaggctctgggtctcgtgaagctggtccagtctcaggtggtggacgtggttcacagcgaggtggtttccagcagggaggaggacaacaccaaggtggaaggggttatactcctcaacctcaacagggaggtcgtggtggtcgtggatatgggcaaccaccacaacagcaacaacagtatggaggaccacaagagtaccaaggaagaggaagaggaggacctcctcatcaaggaggtcgaggagggtatggcggtggccgtggaggtggaccttcttctggaccaccgcagagacaatcagttcccgagctgcatcaagctacctcacctacttatcaagcggtgtcttctcagcctacactgtctgaggtgagtcctacccaggtaccagaacctactgttctggctcagcaatttgaacaactctctgttgaacaaggagctcccagtcaggcaatccagcctataccttcttctagcaaggctttcaagtttccaatgaggcctggtaaaggacagagtggaaagcgttgcattgtgaaggctaaccatttctttgctgaactgcctgataaggatttgcaccattatgatgtgagttttgtgttcccttagtattagctaccatacatctaatgtctatttataaatttacctgaccaactgtgttctttctgttaggttaccattactccggaagttacatcaaggggtgtcaatcgtgctgtgatgaaacaacttgttgataattatcgtgattctcaccttggaagtcgtcttccagcgtatgatggtcgaaaaagtctttacactgctggtccacttccctttaactccaaggagttcagaatcaatcttcttgacgaagaagtaggggctggaggtcaaaggtctagttcttatggtttcttgttgatttttttaataaggaagtttcagaataataacatctttcctattttacagacgagaaagggaatttaaagttgtgatcaagctagttgcacgtgctgatctgcatcacctaggaatgtttttggaggggaaacaatcagatgccccacaggaagctctgcaggttcttgacattgttcttcgtgagctgccgacctctaggtactggttactcatcctatggatgtttgcttctttactaccatgcttgtaagtctcagctggtttagttttgctaacatgttagaatcaggtatattccggtgggccggtccttttattcccctgatataggaaaaaaacaatcattgggggatggcttggagagctggcgtggattctaccaaagcattcgtcctacacagatgggcttatcactcaatattggtgtgatggcttgctgtattacattcaataatttttgatatctggttctccttgaatgtttaactcatggttgtgctgcaattttgtctgttttgtagatatgtcatcgacagccttcatagaggcaaaccctgtgattcagtttgtctgtgatttgcttaaccgggatatttcttctcgacctttatctgatgctgatcgtgttaaggtatgaatcaattgtcattttctgtagtaagtgaaatgttgtattaatctactcttctttgcttagtgacttggaatcttcttctgtcaacagataaaaaaggctcttagaggtgtcaaagttgaagtgactcatcgaggaaacatgcgccggaagtaccgcatttccggtttgactgctgtggccactcgggaattgacgtgcgtatttgttttagatttactactcttatttcgcgaatgttgaacttgatgaaattaatttttactctctgtttttatccttttcagattcccagtagatgaaagaaatactcagaaatctgttgtagaatacttccacgaaacatatggttttcgcattcagcacactcaactaccatgcttgcaagttgggaattctaataggcctaattacttaccaatggaggtaactttgaaatacgtcatcaggttttattagctcaccagtacttccgattctaatgtgttgctgcacaggtatgcaagattgttgaaggccagcggtattccaaaagattgaatgagagacagatcactgctttgctgaaggttacctgtcagcgcccgatagatcgagaaaaagatatcttacaggtatgtttctttattttcgataactttgttttccttcacttgaatcaagagtatctttttgtccgcctatcttgatagtcgttttaaataatgtatgctggtgataaagcctcattgtggaatagatcattgtaaaatctgtttattcttacttggttgcatgtgtttacttgaagtaagtgattttctcaattcatatctaacttgtttttaatcatataaacttgtggctgaaaggatgttatggaatgaatggcttaaaaacctgtttcttctttacttggatgcatatgtttgcttgaagagagtgatttaccttgagatgatgcctttacttcataaatagtgaggcagattaccatcttcagagttgtgctcttaatttagcagcgcgtgtgtcttctggctgctgttgttgaaaactcaattgtttattgctaacattatacaatattttctgatttctagacggtgcaactcaatgattatgctaaagataattatgctcaagagtttggcatcaaaataagtacttctctggcttctgttgaggctcgtatactgcctcctccatgggtatagtaatcgttcccattgtctctttgctattacactttttctctgtatcttaatgactcaaattagggttccaatctgattttaatgttctgcagcttaagtaccacgagtctggaagggaagggacttgtctgccacaagttggtcaatggaacatgatgaataaggtaactagacgaaaatgttcctggttttcaactatgcacttacatgtttcttgcttgtgttacctatatatacctatgctgtcatttttatgtgaatattttgtaaatcatggctgagctgtatatttccaggtaattggcaagaaaattgtttatttatttgtctgttatgagacatattactctaaaatgctattctcttgatatgagtatgggtgtggcacttgactagttgcagagagtattttgttcatgtagctatgtagatacacactgaatttgtctttgttattttttgaaagtgatcacaacgttttctgttgtctggctaatatgagtcttctctgcagaaaatgatcaatggtggaacggtgaataattggatctgcatcaacttttctaggcaagtgcaggacaatctagcgcgtacattttgtcaggaacttgctcaaatgtgttacgtatctggcatggtgagtctttggaagcacaaacttgatcagtttattaacctttcctacactgattccttccctttaattttttatttcttacctgcaggcatttaatccggaaccagtcctcccaccagtcagtgctcgccctgagcaagtagagaaggtcttgaagactagatatcatgatgccacatcaaaactctcccaaggaaaagaaattgatctgcttattgtcattctgcccgataataatggatcattatacggtatgagccatggtctcggatgtttcattgtctacttctgcttaggaacacactctcaacttattttccttaataatatccattgcaggtgatttgaaacgcatatgtgagactgaactcggcatagtctctcaatgttgcctgacaaagcatgtctttaagatgagcaaacaatacatggctaatgttgcgctgaagattaatgtgaaggttggaggaagaaacacagtgcttgttgatgctctatctaggcggattcctctagtcagtgatcgacccaccattatatttggtgctgatgttacccaccctcaccctggagaggattcaagcccatctattgctgctgtaagttagcttctcctttaagatctacttctgtatatggatgcttttgtttattttcatggccttaaccttttcaaattgtgcttgttttgttgacaacgaaatttttgttttcaggttgtggcatctcaggattggcctgaaatcactaaatatgctggattagtttgcgctcaagcgcataggcaggagctcattcaggatctgttcaaagagtggaaggatcctcagaaaggtgtggtgactggtggcatgataaagtacgtgtggatgttatttaatattttcgtgattgggttttagctttagcttgctcttaagtaacagatctgtacgtttctccccttacttttgaacagggagttgctcatagccttccgtagatcaactgggcataaaccactaaggatcatcttctacaggtattaactcttcttatggcacttacttgactaaataactttctgtctttctttctgaaatgtattatatggttattctgcacagggatggagtcagtgagggacaattttaccaagttttgctctatgaacttgatgccatccgcaaggtaacagttacttctaacaaatggcttgtgttgcgtgctcgttttcttctgtttaatctgattattatttcaactcatttcaggcctgtgcttcgctggaagcaggttatcaaccaccagtgacatttgtggtggtgcagaagcgtcatcacacgaggctgtttgctcagaaccacaatgatcgccattcggtggacagaagtgggaatattttacctggtgagacaagtttggatctttgtcaagctttttgtgcgtagacctaatcagctgaaaatcccttgcattggtttttaacccctttgttaattgtaggcactgttgtggactctaaaatctgccaccctacagagtttgacttttacctctgtagtcatgctggtattcaggtataatttacaatctcaaatgagtttgtgggtgagaatttatttgaagtgtttaacaaatcaaaactccattaacagggcacttctcgacctgctcattaccacgttctttgggatgagaacaactttactgcagatggacttcaatctctgaccaataacttatgttacacgtaagatttcttacctttgtaacatattccttgttacaagtgtttgcaagattgcaaggaaaataagagtaacatttaaactacctttttgatttgtacaggtatgcaagatgcacacgctcagtttcaattggtaagttttgtgtttcccatcccttaatgtcatcttgattttgatataaagaagagagagatttagtattcaatgttttttttttttgttttgggatattgtagttccccctgcatattatgcacatctagcagcttttagggctcgattctacatggagccagagacatcagacagtggctcaatggctagtgggagcatggcacgtggaggtggaatggctggtagaagcacacgcgggcctaatgtcaatgctgcagtgaggccactcccagctctgaaagagaatgtgaagcgtgtcatgttctactgctgagttgattcaccctctatctatctttatgacctaaattaatgaagaatatcatgtatgctttctaagacttatcgtgtgtttggatatttcatcactctttctctatgagtatgagatgctttatgactcttgtttgacaactactaaaatttattattcaaaacagactttgatcctttctttaactcgtcttaaatctttattcaagagattttgtgtttagagatgtttaggttgtttgtattgtcgaccacactactacattgtaggtataagcgttgagatccaggacctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 90 - DNA, 14718 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccgaagaaaaaactcatcatcatcatcacagtaccaacaaacacatccctagttccaaatcaagaacacctcttctccacaagccttatcatcaccatgttcaaaccaatcctcctccttttcttttgcacccttcttctcaccaaaacctcaatcttgttgcctcaaatcttccttctagctactactactattactactgctacttctactctcaatttcacaactctcttcctcctcctcctcctcctcatcttcttcctctttctcctcctcttcctcctttacttcctcttcctcctcctcactcaatgacccgtttccacaaatctctccctgtttctcaaggtgactactaatctacctttttcttcttcttggtttattctttgcatgtttggtctaaaggtttaggatcctcacctatggctgaattagtttatatttattggacgaggttttcaaggctatctcttgttgttcttgttttgctgtgtattcccacacaaactctagtatagtgtaaatgtctaaatggtccttctccttctctgtagtctgtacaatattcactttactttaaatgttctctttctttaatgtgttaatgtctgaaaacaaactctctgtctccctttaatactctctctattggtactgatttacttgcatctttttgtgtttcattcaaaaacaattaaagtctaaatttgactttgactcacccatttttttgcttcaacagttgtggagagaaagcagcaacaccaacagaagaagaagatacaagtttctaacaacaaagtgagtggatccatagcaatagaagaagcagcattagtggttgctaaaagaccggactttggtggtcaagatggttctgtcatttatctcctcgccaaccattttcttgtcaagtttgattcgtcacagaggatttaccattacaatgttgagatatctccacagccttcaaaggaaattgctagaatgatcaaacagaagcttgttgagacagatcgtaatagcttctctggtgttgttccggcttttgacggcaggcaaaacatttacagccctgtggagtttcagggagataggcttgagttctttgttaatctccctatcccatcttgcaaggctgtgatgaattacggtgacttgcgtgagaagcaacctcagaagaagattgagaaactgtttagggttaatatgaagcttgtgtccaagtttgatggtaaagaacagagaaaggaaggagaggattgggctcctctgcctccagaatacattcatgctcttgatgttatcttgagagagaatccaatggagaagtgtacatcgatcggaagatcgttttactcgagttctatgggtggatctaaggagattggaggaggagctgttggactcagagggtttttccagagtcttagacatactcagcaaggtttagcacttaacatggatctctcaatcacagctttccatgaaagtattggagtaatagcttacttgcagaagcggctcgagtttcttacggaccttcctaggaacaaaggtagagaattgagtttagaggaaaagagagaagtggagaaagcacttaagaacataagagtctttgtttgccatagagaaacagttcaaaggtatcgagtttatgggttaacagaggaaattacggagaatatatggtttcctgatagagaagggaaatatctaaggcttatgagttacttcaaagatcattatggttatgagattcagttcaagaacttgccgtgtctgcaaatcagtagggcaagaccttgttaccttcctatggagctatgcatgatttgtgaaggtcaaaagtttcttggaaagctatcagatgatcaagctgcaaagatcatgaaaatgggctgccaaaaacctaatgaaaggaaagctattattgataaggttatgacaggatcggtcggtccatcgaggtaattcaaataatctgagcttctatattcgagtactaaaacatcgaaaagagtttaatattttggttctgtactttgacagcggaaaccaaacaagagaattcaaccttgaggtttcaagagaaatgacattgctgaaaggaagaatacttcagccaccaaagcttaaacttgaccggccaaggaaccttaaagaaagtaaagtttttaaaggaactagaattgagagatgggctttgatgagtattggaggcagctctgatcagaaatctaccattcccaagttcataaacgagcttactcaaaagtgtgagcatttgggagtcttcttaagcaagaacacattaagcagtaccttctttgaaccatcacacatactcaacaacatttcgcttctcgaatcaaaactgaaggagattcaaagagcggcatcgaacaatctccagctgattatttgtgtaatggagaaaaaacataaagggtacggagatctaaagaggatatcagagacaagaattggtgttgtgacacaatgctgcttataccctaacatcactaagctcagttctcagttcgtttcgaacttagctctcaagataaacgccaagatcggtggatccatgaccgagctctacaactcgataccttctcacatccctagactgcttagacccgatgagccggttatcttcatgggagctgatgtaacgcatcctcatccattcgatgattgtagcccttcagtagcggctgtggtagggagcataaactggccagaagctaaccgatacgtctcaagaatgaggtctcagactcataggcaagagatcatacaagatcttgacttgatggtcaaggaacttcttgatgatttttacaaagcggtaaaaaagcttccgaatcgaatcatattcttcagggacggtgttagcgagacacagttcaagaaagttctccaagaagagcttcaatcgataaaaactgcttgttcgaagttccaagattacaatccaagcatcacattcgccgtggttcagaaaagacaccacacaaggctgttccggtgcgatccagaccatgagaacatacctcctggtacagtggttgatacagtgataactcatccgaaagagtttgatttttatctctgtagccatttaggagtgaagggcacgagcaggccaacgcattaccatattctatgggacgagaacgagttcacttcagacgaattgcagagacttgtgtataatttgtgttacactttcgtgaggtgcacgaaacctatatcgattgtgccaccggcttattatgctcaccttgctgcgtacagaggaaggctatacatcgagagatcatctgaatctaatggaggatccatgaatccttcttctgtgtctcgtgttggtcctccaaagacgattcctcttcctaaattaagtgataatgtcaagaatctcatgttttactgctgattctttaatatataaaaaaattgatcttgatactgttatctttataggttccaatgacaaattcatcacatattatcataagtactaccaaagccaattattgaacctagaaagtccaagccagtgagtttttattgtatttttctgtaattcataatttctttaaagtaaaagttctttttgtttttttgtttttttcttgaatgcgtgaagttgatgttgatcacctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 91 - DNA, 15585 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccgatacgactctaccgcctcctcaacacatggaacgtgaacctttgaagagcaagagtagtcttcttccaatgactcgccgtggcaacggttccaaaggacagaagattcttctgctcactaaccacttcagagtcaacttcagaaaaccaaacagtcataatttcttccattacagcgtgcgctttttgtttcccttcctgttcctgtttccatgacttgatttggaaaacatactttatagtgtgctttgtctctttggatacaggttactatcacatatgaagatggtagtccacttttagccaaaggttttggcagaaagattcttgaaaaagttcaacagacttgtcaagctgatttgggttgcaaacattttgcttatgatggcgacaagaatctttatactgttggtcctcttcctagatccagtctagacttctctgttgttcttgaaaccgcgccttctaggagaaacgcagacaaaagactaaagcttcctcaccaatccaagaaattcaatgttgcaatcctttttgcgcctccagaaatcccaatggaagctattgcaaatgctcttcaaggaaagaaaactaaacatcttctagatgctatcagagtgatggactgtattttgagccaaaatgcagctagacagttagtgaaccttttttagtttttttttcttagatatattcttggatatttgatgttcttgatgttcatgttgtgacacatttctattttgcgcagaggttgcctcctagttcggcaatctttcttccacaatgacgcaaagtactttgcgaatatcggtgaaggtgtagattgttgcaaaggatttcattcaagcttcagaactactcagggaggcttgtccctcaatattggtaatttcttttcttttaacatatgttttttcatgtggtattaaaaacattctttgtaacatttcagttttaatttctttcatcagatgtttcgactgctatgatagtaaaacctggtcctgttgttgattttcttattgcaaatcaaggtgtgaacgatccattctctattaactggaaaaaggtatttacaagaatttcaaatggtgttttagcatatctttgaggtgaagtacttcattttctgatttttttttttgtttctctctcgtataggctaaaaatactctgaaaaatcttagagttaaagtcctcccttcaaatcaagaatacaagataaccggactaagtggactacactgcaaagatcaaacgtatgttgtgagttgatatttattttaagttttcttgttttgcttctcgtttatctctttttcttgtgtgatttaggtttacttggaagaaaaggaaccaaaacagggaatttgaggaggttgagattacagtgtccgattacttcactaggatccgtgaaatcgaactgcgttactcgggtggcttaccttgtatcaatgttggtaagccaaatcgtcctacctactttcccattgaggtaatatttgttttttgttttgtttttcttccacataatctcatgtttaatggcttatgatacttatgatactttcccatcttttgttttataaaacattttgcagctctgtgagcttgtatctctacaacgctatactaaagcgctaaccaaattccagaggagtaaccttatcaaagaatcaaggcagaatccacaacaaagaattggtgtgttaaccagagtaaggactttaaattttccatttatcctctttttatttgcatttttatgtatactaagttccatgttttgcaggctctcaagactagtaattataatgatgacccaatgttgcaagaatgtggtgttaggattggctctgacttcactcaagtcgaaggccgcgtgttaccaacaccaaaggtgatttttcttccattacatttttggttatggaaggtttgattttattattatatatctgtggtctcttaatcccttataaacagcttaaagctggcaaagaacaagacatctatcctattaatgggagttggaacttcaagaacaaggtccttaactatcctctgtcttctcaaacaagttttgttttgaaaacttgtgacactgatcatcaaactttcatctacagacattttttaagccagccacagttactagatgggctgttgtgaacttctctgctcgctgtgacccgcaaaagattattgatgatttgaccagatgtggaaagatgaaaggaattgtaagcttacttttcgtcatcaggaccattggttactacaataaagaacactaataacaaaaccctctcttggtttttttgttttgttttcattaccagaacgtagattctccataccatgtcgtctttgaagaaaatcctcagtttaaggatgccactggttctgtaagagttgataagatgtttcaacatttacaatcaatactcggcgaagttcctccaaaattccttctatgcatactcgaaaagaaaaactctgatgtttatggtaacactaagcacttaacctttcctgaatcatatagtatacaccttttttttttttctcatatggaaaattttatcacttaggtccttggaaaaagaaaaatcttgttcaatgtggaattgtgaatgtattgttcctcctcaaaacttaaatgatcagtatctcacaaatcttctactaaagataaatgccaaggtgatgatctcttcttgaatcaatctttgtagtttctgatataataatcttatagttaatctctgccatttctgtatagcttggtggattgaattcagttttggatatggagctgtcaggaacaatgcctctggtaatgagagttcctaccatcattattggaatggatgtatctcatggttctcctggacagtctgatcatataccatccattgccgcggtaacattgtttttttttttctttttcactttcttgtgttgtatttttatgtaagctactgatatctttggaatgcataggttgtgagctccagagagtggccactgatctcaaaatacagggcttgtgtgcgtacacagtcgcctaaagttgaaatgatcgatagcctctttaaacccgtctctgacaaagatgatcaaggtatcatgaggtaatactatatattactggactattttgtaactcgtattaattagccctgcttttatgtaacagtatatgatttttaagattcaatgatgtgtttacagagagctcttgcttgactttcactcaagttctggaaagaaaccgaatcacattatcattttcaggtatgcatgtataatttttaggcatgcatgtagtttaatacaatttttctaactcttaactacaccaatacatgcagggatggtgtcagtgaatctcagtttaaccaagttcttaatattgaactggatcagatgatgcaagtaaggattttggttttaccatttttaagctcttaaatacttagcctatgtttgaccattttggagtttaaggtttaacttttgttacattattaggcatgcaagttcttggatcagaagtaagatcccaagtttacactgatcatagctcagataaaccaccacacgaagttcttccaaaccgaaagccctaataatgttcttccaggtaaacagttgcagatatttttttgtgttttgtttgtttgttagtgttgttgaaatgacttgaaacctgttctctatttacccgcaggaacaataattgacagcaacatctgtcaccaacacaacaacgatttctatctttgtgctcatgctggaaagattgtaagtgataagtatatatattattatgctcgaatcctttatgaatcgaacaaaaacttaaaaacacttttcgatgtgttagggaactacaaggccaacacattaccatgtgctctacgacgagattggatttgacacagatcaactccaagaacttgtgcattcactatcctatgtgtaagcagcacatcacttcaaacttttttttacctgatacattaaatctctgtattgaatttagccattaatctcttaattctttgctttattacttgtagctaccagcggagcacaactgccatctctcttggtaattcttttggcatttgatatctcagctttatgtgtagaatttgaaaaattgacacaacttgattattatgcagttgcgccgatatgttatgctcatttggcggctgcacagatggcaactgcaatgaagtttgaggacatgtctgagacttcatcgagccacggcgggatcaccacagctggagcagtccctgtgcctcccatgccgaagctgaacaccaacgttgctagttcaatgttcttttgctgaaagaacaaaaataagcacaagtgacttggcttttcttctaatgaacaaaccaaataacaagattgtttctctcgctttcatgacttttcgtttattctctattttattgttgattgctgttttcgattcttattgaatcgcaaacttaagacataatcagttgccctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 92 - DNA, 16129 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccatgaccaccactatggattataacagcagcgatcaaggtttttcgtggtcagaaattgctcttctgggtagtgttgaaacaatgttggagaaagtctacgggaaacacaatcatcatcctccgataaaagtggagacaagacggagactttcttcgatttctgaggaattggctttggaaacactcaggaaagttctcaatatgccgaatgtgaagaccctcgatgggataatcatatattttcttaacgatgcggtcaccgttgatggctctcctcgcctgtggtctggcgagtctccagtacagtttcctagaactcccgggaaaaaaagttgcagggcttctcaaggtcagtgttaggatagaatctgttcacatactttgggtttttttttttccttcttgtgtgtttagtagtgaaattctcttcttgttgaccgcaatattatgactatggttgattgtttgtagcagaggtgtctcttgatcgtgaagatccgtctcctaagttcttgagaggagatgaaaatggagaatctaagcacatttctcttctattagctttgggtgaacttgaattcaagaaggcgtttttgttgcttacctatcttggagggtatgtttaggctggagatttttccatcagttttatatctatttcgttggaaagtgatcgtctttgtaacaggaaatctctgggtgaggttatatctggtgatgagatcagacaatggaaggatttgccaatggttgcatatgaaagagcagtgtggttcaagttgggccaaaacgaggaaagaatggtatggcagttctctgttatgttctatattggtgctatctatcatagtttttcttaaatgaattgacaaaagacatggagagaggcttgttggatactgcaaattcttttgaactgcttgctgcactgcaatttctgaggaattagactttgttgtcgaggttgtactaattgcttcttcttacatgcttcttttcctcagcagcttgaatcggatagcgggaagactcattactatcaatgtcatgttgctcccgatggtagctacagattaaaggtccaatcggctctccacccactgtcatgcgtgttctttgttttaagatgatctgaattcaagtgatacttttaggatataatgtttattattgataagactgtctttacagggttattttctggagaacactggaacacacctgcacaaggttttgggtgatgataatgttttaaccgttagattcgatcaattaccaaaggagtcaacatactgtgacaacccttactctaaatacaaagagattgctaagaatggtatcatggttggtttacgtcgttatcaattttttggtgggtcattttccttttctcttgattatcatttatgtttttgcaaaaacaacacttttccttgttaaggtgctaaggccatgtctagcagatattgtcatactttatcacatagtaccttgttaacttcttttaaacagtcttcaaagatggagggaaagcagagaaaaagaagagaaattctacaaagcaagtgaaatgctacttcatacgcacgggttctacagcatccagtgatatggagaatccttatatcctttccgggatgtcaattcatgaagctcgtatgcattttatgcatgtgcatacattgccttctcccgccaactatatggcaaggtactctagtttcttctaagtctctattggaggcatcttgaagatttcttactgtttcattttataactgttttagagatgttttcctttgtaatcagggttacctcacttttgttgatgcttcaggttttctttaattctgtcaaagacaaaaaagcttgaagttgacatgactgagattaccgtcatgcaaatcgatgatatacactgccatgtacctctttaattcatgtgtccttcaggaactgtattcgctatatgggctttgacctctttccctctctgacaattgcaggatcaaagtaataatgatgttcttgataagaatgggaaacctcgtatacactcagatggaactggatacatctctgaggaccttgctcggatgtgtccattaaatatttttaaagggaaatctatgagaagtaacaatattcaggtctttcttgtacttccatgacaatttaatggttgcttagttcactcttgtttccttattatgtttgttttgaatcacagggaacctgtgtccaagagccggtatgtagtttacagttcaagtttaagctcgattctgtatttgattatcacttaccacaagtagtgacactttttttttttattccaacagcctctgctgatccaaattcggatgtttaatgatggctctgctgttaagggaattttcctattaaacaagaacgtaagtagactctctttcatgtcacagaaccagatttttgagctaatcctaaaattgctcccaatttggcagcttcctccacagaccgtccaggttcgaccttctatgatcaaggtgtataaagataaaaatttgtcaaattttagcacgtttaactccctggaggtagtcactacaaggtatatgtctcttggatttatgttatcttaaagctgaacttctcactgaggttcttattttgatgatttatggttgtttccatcttccagtaatccaccaaaaagagcgaagctatcgaagaatttagtcgcgctgctgagctatggaggagtgccaaatgatttctttttggatatattgctcaacacgttggaaaagaagaaaaccatattcttcaaagtacgtgcagcgggtaaaggtatggttaaaactctaaagctttcgagctcttttgcaattagagggaaaccatggtcttttgcaattttcgagctttgaaactgactgaagtctttaatatcataggaagtatgagaagacacatgatggctttttattaagtttatttgtttctgcagctgcacttcattatgggaatatggacgataagaacgctttacaaatgatcatggctggtatcccccttgatgagccatatttgaagcattatctgtctaaactcttaaagttagagaaagatgatctcaaagcaggaaaacttcctattgacgaatcatactatctcatggggacagtggatcccaccggagagctaaaggaagatgaagtctgtgtcatcctgtatgctaccaaaattttatttatttttttgtaatataaagtgtggatggaagaataaacaaaccgtgcaacttgcttcattggagttgtgagttagctcttcatttcctcactactttctttttcgttgttttgcagcgggtctggccaaatctcaggatgtgctagtttacaggaatcctggactacattttggagacatacatattctaaaggctacatatgttaaatccttggaacagtatgttggaaattccaagtatggtgtgttcttccctcaaaaaggtccaagatctttgggagacgaaattgcaggtggtgactttgatggcgatatgtatttcatatccagaaatcccaaggtacactgctatatccatcacagttatagtaaaatttcattgctgagacctaacttataaaccgcaacttcctctgtttagctacttgagcactacaagccaagtgaaccgtgggtgagttcttctccacgtagtaaaatttacactggtagacaaccaagtgaactttcacccgaacagttggaagaagagcttttcaaaatcttcttaaagacaggatttagtcccaggtacattctttaattctcgggttactttcaaggtcacatagtcctttgggttaaatatgtcttgttttctgggtttacagctcagttataggacaggctgcagatagctggctagcaataatggaccggttcctcaccttaggagatgagaatgttaaggaaaaagctgaaatgaagaagaaaatgctaaagttgactgatatatactatgatgctattgatgcaccaaaaacggggactgaggtcaatctcccgttagacgtaaaggtcgatctttttccacattatatggagaggaataaaacattcaagtccacttctattcttggattaatctttgacactgtggattttcataatgcagaggacaccacaccatctggtacgggtcttcttgttactattgataatcaaagtacacaataactcatcacgttgaatatgttttaacggtttagctctgggttacctttcaggaatcagtaaacttcagtgttttgaagatgagccggtctctgagtttgacatggagaaatgtaaactctggcacaaggattacagaaaggagatgtgtcaagcgatgaactcagatgatgatgattcctgtaatgaagttatccagaaatacaagcaggtaagctcttcaatttagttaattctaagagaaaagctttaaccaatagccttcatggatcactggttttttgttgattgtaatgctttataaacaaatcattggttgaacggtttcttctctctgtaacaggagttctacagtgctgcagggtttaaagaaagcaagaaaattctagaagaactctacccaaaggcccttgcactttacaacgtcacttaggactatgccatcaagtggaataacgttaaaagttgcgggtttgtctggaaggttgcaggtccggtcctgtgcagattctacgagaagaatttagtaatgtgttcgatttccatgctgaaagagattttcggctgaacactcttactgttctgttctggtgataacaataagtatttgctgtcttcatgtctcataatatgcgtgttaggcaagtaacctacttataggccaaaccaagatgtgtggtgtagtactggctttgatttaggtctaatcttagatttgtcctctagttaacatataattccgtgtgtatgtatatttgcttatggaatagaaaaaaatgatgacctttatagtgtggtggtgtacaaagctatcttctccagtgcgactcctctctgcgtcctcatgcttccatttcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 93 - DNA, 16263 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccaatcaagcttcttcgaggataaggatcgctctttcgggtcggattgaaacagctttagagaatatctacagaaaacacaatcttactccgattaatgatgagacgagacagagactgtcttcgattcctgagaatttgggttttgagttggtcaggaaagtttttagtttgcaggctggtttgatatacaacctcgacagcttcatcgtttctaaggttaaccaagccgttagttttactggctatccacagccggcgaactctcttagcccctctggaagacacgtgtccagggttcttcaaggttagtatcagaaacaaactctctttgcttgtgaaaatgtgaatttggatagtaaaaatctttacttttcttatgattgttggcgattgcagaagagatgtctgttgattctgatgctccttctcctaagtctttgaaaagtgaagacaaaggaggatctctgcacattccccagctggtagctttgggtgaacttgaatttaagaaggtgtttttgttacttagctatattccagggtaatgtttgttatgcaagtttcccaatttcttacatctctatattctcttatccacctattgtaattgtttggttttgatggtgacaattacctcttttggtgatgcttaactttttctttatttgatgttatacaggcaacatgtgggccaggttataactgctgatgagattagactgtggaaagatttgccaatggttgaatatgaagcagcggtttgggaccgcttgggccggcattattgtccccaaaaggaccgaagaatggtattgttctattctattgggttttgtttatattttcatgtttgcgtttctgaaggtaattttacaagaagttgagtttggtatcaaagttgtattaactgcattttctatcatgcttcatttccccagttgcaatgggatagcgggaagacccattactatcagtgtaatgttgctcccaatggtagctacacattcaaggttctttcagctcttcaagtactatcttttctgtttgctttttccattaggcatatgtactttgtgtaatatttttgattcaagctgtagaagaaattctttgtaagtcgctatcatatgtctataaaacaattgatatgtctatcaaacattgcaccttgacattgactcaatatacttatcgacacactgtgttcacagggccctcttcttgaacacactggaacacacttgcacaaggtcttgggtgatgataatgttttaaccgtcaaattcgcggatgtccagaaaagttcatcaacatatagcattgaccattactttacatacaaagggattgccaagaacggtatcatgattggtttacgtcgttatcaattttttggtgggcattgttttccctttttctcttgattaactaccatttatgtcgttacacatacaaaactatttattgtaatgttgctaagctatacacatatcatacttctgctttagatttagaacctgatattgtagctctttgtgacattctattgaacagttttcaaagatggaggaaaagaggaaaagaagaaagatctttctacaaagaaagtgaaatgctactttatacgcacggactcaacagctttttatgatatgcaaaatccctatattttaaccggaaagtcaatttatgaagctcgtatgcattttatgcacgtgcatcgagcaccaactttggcgaattatatggcaaggtactttaaaggaagtatatgaagatattctactgtatcatatttaaactcttctgtagatgtccttttttatcagagtcacctttcttttgctgatgcttcaggttttccttaattctgtcaaagactaaaacgcttgaagttgacatgactgggattacctttgatcaaattgatgatatacactgccatgtacgtccttaattcatgtttccttgaggaacttcgtctctaatgcctttacttacttgcattcgctatgttctttgacctttttccatttcttccaattgcaggatcaagatgggaaagatgttcttgacaagaacaaaaaaccttgcatacattcagatggaactggctacatctctgaggacctggctcggatgtgtcccctaaatatatttaaagggaaatgtctcagaagtgaaagtattcaggtccttactctgacgttgatggttgactaggccctttgttcactcgtatcatcttttttccttcttcatacttgatttaaatttagagtaagctttcatattttgttggacaggaagcatgttaccaagacccggtatgtatactatatagttcatatccctatttatgcttgattctatgtctgtctattattgaactcaagtagtaaaatattactttttccaacagcctcttttgatccagtttcggatgttttatgatggttatgctgttaagggaacttttctcttaaacaagaaagtaagtccgctctctttcacattacaacatccttttttggggtttaaatgtttgcaaaactattcttaattttctttccaatttggcagctttgtcctcggactgtccaggttcgaccttcgatgatcaaggtgtctaaagatccatccttgtccaattttagcacctttaacgccttggaagtagtaaccacaaggtattttgttcgctgactacttgttgttttaatactgaagttctctccctcaaatgttcttattgtggagtgaagttatcctagtaacatctagttgtttccatcatccagtaatccaccaaaaagaacaaagctatcaaaaaatttggttgcgctgcttagctatggaggaatccctaatgaattctttttggatatattgctcaacacgttggaagagtctaaaagcatattctacaacaaacgtgctgcacttaatggtgcttaaaattcctaacgtttctggttcttttgcattttgtttctggtgcctagagctatttatttttaagaagtcaaattttcggtgcagctgctcttaattatggggaaatggacgaccagaacgccgcacagatgatattggttggtatcccccttgatgaaccacacttgaagaattatctgtctatacttttaaagacagagaaaaatgatctcaaagcaggaaaacttcctgtgaccgaatcatactatcttatgggaacagtggatcccaccggagcgctaaaggaagatgaagtctgtgtaattctgtatgctactcaactcttcttttaccttttcattttgagttttaattgcatggaaaaaaaaaacagtggtttagttttttggaattgtgagttagttgttcatttcgccactgcttcttttcatggttttttagtgagtctgggcaaatttcgggagaagttttagtgtatagaaatcctggattacattttggagacatacatattcttaaggccacatatgtcaaggccttggaagagtatgttggaaattccaagtttgctgtgttcttccctcagaaaggtccaagatccttgggggacgagattgcaggtggtgactttgatggtgatatgtatttcatatcaagaaaccctgaggtaatccaccacattaatcagtaaaattaattattgttgtgacctgattttcttgggtttgctttctggtttagctacttgaaaacttcaaaccaagtgaaccgtgggtgagtttgactcctcccagtaaaagtaactctggtagagcgccaagccagctttccccagaagagttggaagaagaacttttcgaaatgttcctgactgcaggatttcatgccaggtactctcatctctgaaagatataaaagtgtcttggatttgcagaatcaatctttgaaattcatttcctttttcctcgggtttacagcaatgttatagggatagctgcagatagctggttaacaataatggaccgcttcctcatactaggagatgacagggctgaggaaaaagctgaaatgaagaagaagatgcttgagctgattgatatatactatgatgctcttgatgcaccaaagaaaggggataaagtctacctcccaaacaagctgaagcctgacatctttccacattacatggagcgggataaaaaattccagtccacttctattcttggattaatctttgactttgttaaatcacagacaacagaggaaccctcaccatcatctggtaaaggacctttattggttctactatatacatataatcagatcaccaaagaaatcattaatcatgatatgtgttttactgtggtttacaatttagagatcagtaaactcccgtgtttcgaagatgagccggtctccgagtttcatatgcagaaatgtagactgtggtatgataattacagaactgagatgacacaggcgatgaagactgataaggatgaatcagccaatgaagttattcagagatacaagcaggtaaggtcctcaattcgatgtcaccactctggagattcagttaagtctaagagcaaagatctgaccagtagacttcatgaatctccaatgttttgttgattggttccgtggaagtatgtaaccagcaggagacaatcatgcttatcaaagaaatggtttatttgtcgtgtgtcaggagttctatggcgctgcagggtttgaagacagcaagaaaagtctggaagaactctatccgcaagctctcgcactctacaagatcgtttacgattatgccattcatgcaggtgtttcgaaatgcaggtttgtctggaaggtagcaggaccggtactgtgcagattctatctaaataagaaaatgcaggagaaatgtttagtgtgtgcaccatctgtgcttaaagagctttggggttgaagaatacaagcctttgttgttgctacatgtctaataataagaagcgtatgaggcaagtaagttaactaataggtgccaagatgtgtgtgttgtagtgccttttggtttggttttaatgtagatttctcctctggtgaatattttttgtgaatcttttgcctatgaaatgaataaaaatatgttacataacgaagcttttctattaacgaagctcttacctctgcaccagtgttttttgtaagattgaaatttgtaaaataaatgttgagaattttaaacaaaattttgatcgcgagattgtttgtttacttggccctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 94 - DNA, 13050 bp, Artificial Sequence
  Synthetic
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggctcgagatggattataaggacgatgacgataaaggcatccacggggtgccagctgctgcggaagcgaaggaggagaatagggagaaaaacgaagaagaagagagcgtaaagcttttcgtcggacaaattccgaaacatatgtcggaatctcaactcttaacattgtttcaagagttcgctgtcgttgacgaggtcaatatcatcaaggacaagattacacgcgcctcacgaggatgttgttttctgctatgtccatcgagagaagaagcagataagttggtcaatgcttgccataacaagaagacattgcctggggctaacagtctattgcaagtaaagtatgcagatggcgaattagaaaggctagagcataagctatttgttggtatgcttccaaagaatgtctctgaagctgaagttcaatctttattctccaagtacgggaccataaaggatctacagattctaagaggtgctcaacaaacgagcaaaggctgtgcatttctgaagtatgagacaaaagaacaagctgtttccgccatggaatctatcaacggaaaacataaaatggagggttcaactgttcctttagttgtcaaatgggcagacacagaacgagaaagacacacaagaagacttcaaaaggctcaaggaggatctggaggaggatctggaggaggatccgcgactcgtaagttttctgcagagcttacttatgatattgttgtgtttactcttctgtgtatttgaccttaatgggatttgtgggtaatacgcaggtcttcagtttgagaacaactgcgaagttggtgtcttttccaaactcactaatgcctattgcttagtcgctataggaggctctgagaacttttacaggtaccttttttttttttctctgtaaagttttcagtattgtttttgcttcttctatttcacttttttgatgtttttggtttgatgatttgtgtagtgcttttgagtcggagttagcagatgttatccctattgttaagacctctattggaggaacacgaattattgggcgtctttgtgctggttagctttctcaactagctgttatattttcttgcttcaaatcatcgtgattatttcattactttagcgtagtgaatgtggttttgtgtttcttcgtataggaaacaagaatgggcttcttgtacctcatacaacaactgaccaaggtaattttatacttctttcctcagatcgtttctcataacaattaagaattgattgtttaataccagcatgatccagttgtctgagatagagattgtagagtttttggtgaatatcttttatgtaacttgcctgaagcatctactctgcgcctctgcccatacctgtttcatttcagtttatgagtggactttttataactgaataagcttttggtgaagatgattttcctccttaacttgttgatgattttgtaatgttgcaatatttattgagtctcttgttcttttatattagaaatctatataggttttaggtctgtaattggttgcatctcctgagtcctgcttatttttttatgtttactcgtccagagcttcaacacttaaggaacagtctacctgatcaagttgtggtccagcgaattgatgaacgtctgtctgctcttggaaattgcattgcctgcaatgactatgtagctcttgcccacaccgatcttgataaggtaaagattcatgtcatactttatttttctaatgaaaccttttcaaaaacatatactcttttcatgactggcaagaaatggtgaaagaataagtgcgcatttttacaaaagttagcagaaatgtaattaagcctttgtaatcaccatgaatcttatatcttctgatgatgatgataacgataacagagttgtacgtatggcgtttggtttgatgcctctggttcttgtgtttttattttgctttgataggaaaccgaggaaataatagcggatgtacttggcgttgaagttttccgtcagacaattgcaggcaacattctcgtgggcagttactgtgccctatccaacaaaggtggaatggtaagagaatgaaaaacataacttcccttcggaaatggatggcattggcattggtctctcattagaatttgaatgtcttttctcaaataggtccatcctcacacctcagtggaagacttggaagaactctcaacacttcttcaagtccctctcgttgcaggaaccgtgaaccgtggtagtgaagttatcgctgcgggtatgaccgttaatgattggacctctttctgcggttcagacacaactgcgactgagctctctgtcatagacagcatcttcaagctaagggaagcccaacccagctctatcgtcgatgagatgaggaagtcattgatcgatacctatgtttaaattttgtttgtttggatgacacacacatcccaacatcgccaatgtgtcaatgtcagaaaattttcatactctactgtttgcaagaattagctgttttattttatttttgtgtggtttttaacagtttaaaggaaatctgttgatgtctctttgaatctttctgagtaagtgataagaaaattctccttaatactacacacattttcaattttcaatttggggtccacattaggttcaaattttagtggagaagaaaccattggtgaatggataacggtcctaggtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 95 - DNA, 1212 bp, Artificial Sequence
  Synthetic
gagaattaagggagtcacgttatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcactgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagccccaaacacacgctcggacgcatattacacatgttcatacacttaatactcgctgttttgaattgatgttttaggaatatatatgtagagcaagtgtataagcacaagtttcacaggtcgtgatatgattcaattagcttccgactcattcatccaaataccgagtcgccaaaattcaaactagactcgttaaatgaatgaatgatgcggtagacaaattggatcattgattctctttgatacttgtccttatacactcgctctctcttttgtattccaattttcttgattaatctttcctgcacaaaaacatgcttgatccactaagtgacatatatgctgccttcgtatatatagttctggtaaaattaacattttgggtttatctttatttaaggcatcgccatggggggatccactagttctagagcggccgccaccgcggtggagctccagcttttgttccctttagtgagggttaatttcgagcttggcgtaatcatggtcatagctgtttcctgtgtgaaattgttatccgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatc

SEQ ID NO: 96 - DNA, 1213 bp, Artificial Sequence
  Synthetic
gagaattaagggagtcacgttatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcactgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagccccaaacacacgctcggacgcatattacacatgttcatacacttaatactcgctgttttgaattgatgttttaggaatatatatgtagaatacctctctagattgtaatttcacaggtcgtgatatgattcaattagcttccgactcattcatccaaataccgagtcgccaaaattcaaactagactcgttaaatgaatgaatgatgcggtagacaaattggatcattgattctctttgatattacattctagagaggcattctctcttttgtattccaattttcttgattaatctttcctgcacaaaaacatgcttgatccactaagtgacatatatgctgccttcgtatatatagttctggtaaaattaacattttgggtttatctttatttaaggcatcgccatggggggatccactagttctagagcggccgccaccgcggtggagctccagcttttgttccctttagtgagggttaatttcgagcttggcgtaatcatggtcatagctgtttcctgtgtgaaattgttatccgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatct

SEQ ID NO: 97 - protein, 170 aa, Artificial Sequence
  Synthetic
SVKLFVGQIPKHMSESQLLTLFQEFAVVDEVNIIKDKITRASRGCCFLLCPSREEADKLVNACHNKKTLPGANSLLQVKYADGELERLEHKLFVGMLPKNVSEAEVQSLFSKYGTIKDLQILRGAQQTSKGCAFLKYETKEQAVSAMESINGKHKMEGSTVPLVVKWADT

(19) United States
(12) Patent Application Publication
(10) Pub. No.: US 2025/0051788 A1
(43) Pub. Date: Feb. 13, 2025

(54) COMPOSITIONS FOR RNA-PROTEIN TETHERING AND METHODS OF USING

(71) Applicants: Donald Danforth Plant Science Center, St. Louis, MO (US); The Curators of the University of Missouri, Columbia, MO (US); Ohio State University, Columbus, OH (US)

(72) Inventors: R. Keith Slotkin, St. Louis, MO (US); Diego Cuerda-Gil, St. Louis, MO (US)

(21) Appl. No.: 18/564,982

(22) PCT Filed: May 24, 2022
(86) PCT No.: PCT/US2022/030789

§ 371 Date: May 16, 2024

Related U.S. Application Data
(60) Provisional application No. 63/192,473, filed on May 24, 2021.

(51) Int. Cl. C12N 15/82; C12N 9/22; C12N 15/11; C12N 15/113; C12N 15/90

(52) U.S. Cl. CPC C12N 15/8213; C12N 9/22; C12N 15/11; C12N 15/113; C12N 15/902; C12N 2310/14; C12N 2310/20

(57) ABSTRACT

The instant disclosure provides engineered proteins, constructs for expressing the engineered proteins, and methods of using the engineered proteins and constructs for tethering a protein of interest to an RNA molecule.

### CROSS-REFERENCE TO RELATED APPLICATIONS

This application claims priority from Provisional Application No. 63/192,473, filed May 24, 2021, the entire contents of which are hereby incorporated by reference.

### GOVERNMENTAL RIGHTS

This invention was made with government support under NSF MCB 1608392 and NSF MCB 1904326 awarded by the U.S. National Science Foundation. The government has certain rights in the invention.

### SEQUENCE LISTING

This application contains a Sequence Listing that has been submitted in ASCII format via EFS-Web and is hereby incorporated by reference in its entirety. The ASCII copy is named 077875-729824_Sequence_Listing_ST25.txt, and is 506 kilobytes in size.

### FIELD OF THE INVENTION

The instant disclosure provides engineered proteins, constructs for expressing the engineered proteins, and methods of using the engineered proteins and constructs for tethering a protein of interest to an RNA molecule.

### BACKGROUND OF THE INVENTION

The post-transcriptional regulation of gene expression is an important element in controlling both normal cell functions and development of all organisms. In all living cells, proteins bind to RNAs and modulate their stability, location and translation. The sorting of RNA transcripts dictates their ultimate post-transcriptional fates, such as translation, decay or degradation by RNA interference (RNAi). This sorting of RNAs into distinct fates is mediated by their interaction with RNA-binding proteins. RNA-binding proteins bind to single- or double-strand RNA in cells. They can contain various RNA-binding structural motifs, such as RNA recognition motif (RRM), dsRNA-binding domain, zinc finger and/or other motifs. Although RNA-binding proteins can be identified easily from a genome (by the presence of a predicted RNA-binding protein motif), determination of the function of RNA-binding proteins remains laborious. Functions of individual RNA-binding proteins (for example: promoting degradation or translation, altering localization, performing quality control, etc.,) are only known for a small handful of well-studied proteins, while the function of thousands of RNA-binding proteins remains enigmatic. Very few techniques have been developed for determining the function of proteins that interact with RNA, and most of these assays are in vitro and require heterologous expression and purification of individual proteins. Few techniques assay RNA-binding protein function in vivo, which is best to identify the protein's role in a living cell. To determine function, most researchers investigate cells in which the protein of interest is mutated, as few other options are available. While hundreds of RNA-binding proteins have been identified, which act to sort RNAs into different pathways is largely unknown. Particularly in plants, this is due to the lack of reliable protein-RNA artificial tethering tools necessary to determine the mechanism of protein action on an RNA in vivo. Further, none of the developed techniques routinely or specifically works in plant cells.

Accordingly, there is a need in the art for systems, methods and techniques that can be used to modulate and study post-transcriptional regulation of RNAs.

### SUMMARY OF THE INVENTION

One aspect of the instant disclosure encompasses an expression construct for expressing an engineered protein for tethering a protein of interest to a target ribonucleic acid (RNA) molecule. The expression construct comprises a promoter operably linked to a nucleic acid sequence encoding the engineered protein, the engineered protein comprising an RNA-binding polypeptide fused to a protein or polypeptide of interest. The nucleic acid sequence encoding the engineered protein comprises a first nucleic acid sequence encoding the RNA-binding polypeptide and a second nucleic acid sequence encoding the protein or polypeptide of interest upstream or downstream of the first nucleic acid sequence. Alternatively, the nucleic acid sequence encoding the engineered protein comprises a first nucleic acid sequence encoding the RNA-binding polypeptide, and a second nucleic acid sequence comprising a cloning sequence upstream or downstream of the first nucleic acid sequence, whereby a third nucleic acid sequence encoding the protein or polypeptide of interest cloned into the cloning sequence generates the nucleic acid sequence encoding the engineered protein. In some aspects, the RNA-binding polypeptide comprises an RNA-binding domain of an RNA-binding protein, wherein the RNA-binding domain recognizes and specifically binds an RNA recognition sequence in the target RNA molecule, to thereby tether the protein of interest to the target RNA molecule.

The polypeptide of interest can comprise an affinity polypeptide that binds an epitope on the protein of interest. The RNA-binding domain can be selected from an RNA recognition motif (RRM), a helix-turn-helix domain, a leucine zipper domain, a winged helix domain, a winged helix-turn-helix domain, a helix-loop-helix domain, an HMG-box domain, a White-Opaque Regulator 3 (Wor3) domain, an OB-fold domain, an immunoglobulin fold, a B3 domain, a TAL effector, a dsRNA-binding domain, a zinc finger domain, zinc knuckles, a KH domain, and any combination thereof.

In some aspects, the RNA-binding domain comprises one or more RRMs of an RNA-binding protein. In one aspect, the RNA-binding protein is a Bruno-like RNA-binding protein. The Bruno-like protein can be a BRN1 protein comprising an amino acid sequence comprising about 75% or more, at least about 85% or more, at least about 95% or more, or 100% sequence identity with an amino acid sequence of SEQ ID NO: 30, and can be encoded by a nucleic acid sequence comprising about 75% or more, at least about 85% or more, at least about 95% or more, or 100% sequence identity with a nucleic acid sequence of SEQ ID NO: 31 or SEQ ID NO: 32.

The RNA-binding polypeptide can comprise an amino acid sequence comprising RRMs A-B of the Bruno-like RNA-binding protein and wherein an amino acid sequence comprising RRM C of the Bruno-like RNA-binding protein is absent. In some aspects, the RNA-binding polypeptide comprises an amino acid sequence comprising about 75% or more, at least about 85% or more, at least about 95% or more, or 100% sequence identity with an amino acid sequence of SEQ ID NO: 33. In some aspects, the RNA-binding polypeptide is encoded by a nucleic acid sequence comprising about 75% or more, at least about 85% or more, at least about 95% or more, or 100% sequence identity with a nucleic acid sequence of SEQ ID NO: 34.

The RNA-binding polypeptide can recognize and specifically bind an RNA sequence comprising one or more Bruno-like protein binding site on the target RNA molecule. In some aspects, the Bruno-like RNA-binding protein is BRN1 and the RNA-binding polypeptide recognizes and specifically binds an RNA sequence comprising one or more BRN1 binding sites on the target RNA molecule. The RNA sequence comprising the one or more BRN1 binding sites can comprise about 75% or more, at least about 85% or more, at least about 95% or more, or 100% sequence identity with a nucleic acid sequence of SEQ ID NO: 5.

The engineered protein can further comprise an amino acid sequence comprising an affinity tag fused to a second terminus of the RNA-binding polypeptide. The amino acid sequence comprising an affinity tag can comprise an amino acid sequence of a maltose binding protein tag, a polyhistidine tag, a Flag tag, a Myc tag, an HA-tag, a Nus tag, or any combination thereof. In some aspects, the affinity tag is a flag tag. When the affinity tag is a flag tag, the flag tag can comprise an amino acid sequence comprising about 75% or more, at least about 85% or more, at least about 95% or more, or 100% sequence identity with an amino acid sequence of SEQ ID NO: 26 and can be encoded by a nucleic acid sequence comprising about 75% or more, at least about 85% or more, at least about 95% or more, or 100% sequence identity with a nucleic acid sequence of SEQ ID NO: 27. In some aspects, the flag tag is fused at the N-terminus of the RNA-binding polypeptide.

In some aspects, the engineered protein further comprises a linker polypeptide linking the flag tag to the RNA-binding polypeptide. The linker polypeptide can comprise an amino acid sequence comprising about 75% or more, at least about 85% or more, at least about 95% or more, or 100% sequence identity with an amino acid sequence of SEQ ID NO: 22 and can be encoded by a nucleic acid sequence comprising about 75% or more, at least about 85% or more, at least about 95% or more, or 100% sequence identity with a nucleic acid sequence of SEQ ID NO: 23.

The engineered protein can further comprise a flexible protein linker for linking the RNA-binding polypeptide to the protein of interest or to the affinity polypeptide. In some aspects, the flexible protein linker comprises an amino acid sequence comprising about 75% or more, at least about 85% or more, at least about 95% or more, or 100% sequence identity with an amino acid sequence of SEQ ID NO: 24, and can be encoded by a nucleic acid sequence comprising about 75% or more, at least about 85% or more, at least about 95% or more, or 100% sequence identity with a nucleic acid sequence of SEQ ID NO: 25. In some aspects, the flexible linker is fused at the C-terminus of the RNA-binding polypeptide.

In some aspects, the engineered protein comprises a flag tag, a linker polypeptide linking the flag tag to the N-terminus of an RNA-binding polypeptide comprising one or more RRMs of a Bruno-like protein, a flexible protein linker linking the RNA-binding polypeptide to a protein of interest. The engineered protein can comprise an amino acid sequence comprising about 75% or more, at least about 85% or more, at least about 95% or more, or 100% sequence identity with an amino acid sequence of SEQ ID NO: 3 and can be encoded by a nucleic acid sequence comprising about 75% or more, at least about 85% or more, at least about 95% or more, or 100% sequence identity with an amino acid sequence of SEQ ID NO: 4. In some aspects, the cloning sequence comprises a nucleic acid sequence of SEQ ID NO: 19. In some aspects, the promoter comprises a nucleic acid sequence comprising a UBQ10 promoter and the promoter can comprise a nucleic acid sequence comprising about 75% or more, at least about 85% or more, at least about 95% or more, or 100% sequence identity with a nucleic acid sequence of SEQ ID NO: 20.

The construct can further comprise a terminator sequence. In some aspects, the terminator comprises a nucleic acid sequence comprising an OCS terminator. The OCS terminator can comprise a nucleic acid sequence comprising about 75% or more, at least about 85% or more, at least about 95% or more, or 100% sequence identity with a nucleic acid sequence of SEQ ID NO: 21.

In some aspects, the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, a nucleic acid sequence comprising a cloning sequence downstream of the nucleic acid sequence encoding the RNA-binding polypeptide, and an OCS terminator. The engineered protein can comprise an amino acid sequence comprising about 75% or more, at least about 85% or more, at least about 95% or more, or 100% sequence identity with an amino acid sequence of SEQ ID NO: 3 nd can be encoded by a nucleic acid sequence comprising about 75% or more, at least about 85% or more, at least about 95% or more, or 100% sequence identity with a nucleic acid sequence of SEQ ID NO: 4. The cloning sequence can comprise a nucleic acid sequence of SEQ ID NO: 19. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20. In some aspects, the OCS terminator comprises a nucleic acid sequence comprising about 75% or more, at least about 85% or more, at least about 95% or more, or 100% sequence identity with a nucleic acid sequence of SEQ ID NO: 21. In some aspects, the expression construct comprises a nucleic acid sequence comprising about 75% or more, at least about 85% or more, at least about 95% or more, or 100% sequence identity with a nucleic acid sequence of SEQ ID NO: 18.

In some aspects, the protein of interest is an RNA de-adenylase protein, an RNA processing protein, an RNA translation regulation protein, an RNA helicase, an RNA transport protein an RNA decay protein, an RNA localization protein, a reporter protein, or any combination thereof. For instance, the protein of interest can be CAF1, RDR6, RDR3, SGS3, SDE5, SDE3, AGO1, AGO7, AGO8, RDR3, RDR4, RPS6, EIF6A, or any combination thereof. In some aspects, the protein of interest is a de-adenylase protein. The de-adenylase protein can be a CAF1 protein comprising an amino acid sequence comprising about 75% or more, at least about 85% or more, at least about 95% or more, or 100% sequence identity with an amino acid sequence of SEQ ID NO: 6 and can be encoded by a nucleic acid sequence comprising about 75% or more, at least about 85% or more, at least about 95% or more, or 100% sequence identity with a nucleic acid sequence of SEQ ID NO: 7.

In some aspects, the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, and a de-adenylase protein fused to the C-terminus of the RNA-binding polypeptide, and an OCS terminator. The engineered protein can comprise an amino acid sequence comprising about 75% or more, at least about 85% or more, at least about 95% or more, or 100% sequence identity with an amino acid sequence of SEQ ID NO: 8 and can be encoded by a nucleic acid sequence comprising about 75% or more, at least about 85% or more, at least about 95% or more, or 100% sequence identity with a nucleic acid sequence of SEQ ID NO: 9.

The UBQ10 promoter can comprise a nucleic acid sequence comprising about 75% or more, at least about 85% or more, at least about 95% or more, or 100% sequence identity with a nucleic acid sequence of SEQ ID NO: 20 and the OCS terminator can comprise a nucleic acid sequence comprising about 75% or more, at least about 85% or more, at least about 95% or more, or 100% sequence identity with a nucleic acid sequence of SEQ ID NO: 21. In some aspects, the expression construct comprises a nucleic acid sequence comprising about 75% or more, at least about 85% or more, at least about 95% or more, or 100% sequence identity with a nucleic acid sequence of SEQ ID NO: 16.

In some aspects, the protein of interest is a translation enhancer protein. The translation enhancer protein can be an RPS6 protein comprising an amino acid sequence comprising about 75% or more, at least about 85% or more, at least about 95% or more, or 100% sequence identity with an amino acid sequence of SEQ ID NO: 11 and can be encoded by a nucleic acid sequence comprising about 75% or more, at least about 85% or more, at least about 95% or more, or 100% sequence identity with a nucleic acid sequence of SEQ ID NO: 12.

In some aspects, the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, and a translation enhancer protein fused to the C-terminus of the RNA-binding polypeptide, and an OCS terminator. The engineered protein can comprise an amino acid sequence comprising about 75% or more, at least about 85% or more, at least about 95% or more, or 100% sequence identity with an amino acid sequence of SEQ ID NO: 13 and can be encoded a nucleic acid sequence comprising about 75% or more, at least about 85% or more, at least about 95% or more, or 100% sequence identity with a nucleic acid sequence of SEQ ID NO: 14.

The UBQ10 promoter can comprise a nucleic acid sequence comprising about 75% or more, at least about 85% or more, at least about 95% or more, or 100% sequence identity with a nucleic acid sequence of SEQ ID NO: 20 and the OCS terminator can comprise a nucleic acid sequence comprising about 75% or more, at least about 85% or more, at least about 95% or more, or 100% sequence identity with a nucleic acid sequence of SEQ ID NO: 21. In some aspects, the expression construct comprises a nucleic acid sequence comprising about 75% or more, at least about 85% or more, at least about 95% or more, or 100% sequence identity with a nucleic acid sequence of SEQ ID NO: 15.

In some aspects, the RNA molecule comprises a messenger RNA (mRNA) molecule. The mRNA molecule can comprise one or more binding sites of a Bruno-like RNA-binding protein in a non-coding region of the mRNA molecule, internal to the coding sequence, or any combination thereof. In some aspects, the mRNA molecule comprises one or more RNA-binding sites of a Bruno-like RNA-binding protein in an untranslated region (UTR) of the mRNA.

In some aspects, the target RNA molecule comprises one or more binding sites of a Bruno-like RNA-binding protein in a 3′ untranslated region (3′ UTR) of the mRNA. The nucleic acid sequence comprising the one or more binding sites of a Bruno-like RNA-binding protein can comprise a nucleic acid sequence comprising the 3′ UTR of a plant SOC1 mRNA and the nucleic acid sequence comprising the 3′ UTR of the plant SOC1 mRNA comprises a nucleic acid sequence comprising about 75% or more, at least about 85% or more, at least about 95% or more, or 100% sequence identity with a nucleic acid sequence of SEQ ID NO: 37.

In some aspects, the mRNA molecule comprises a plant SOC1 mRNA. The SOC1 mRNA can comprise a nucleic acid sequence comprising about 75% or more, at least about 85% or more, at least about 95% or more, or 100% sequence identity with a nucleic acid sequence of SEQ ID NO: 35 or is encoded by a nucleic acid sequence comprising about 75% or more, at least about 85% or more, at least about 95% or more, or 100% sequence identity with a nucleic acid sequence of SEQ ID NO: 36.

In some aspects, the mRNA comprises a chimeric mRNA comprising a nucleic acid sequence comprising one or more binding sites of a Bruno-like RNA-binding protein cloned in a 3′ UTR of the mRNA. The chimeric mRNA can encode a Cas9 protein of a CRISPR/Cas programmable nucleic acid modification system. In some aspects, the chimeric mRNA comprises a nucleic acid sequence comprising one or more binding sites of a Bruno-like RNA-binding protein. The chimeric mRNA can comprise a nucleic acid sequence comprising one binding site of a Bruno-like RNA-binding protein. The a nucleic acid sequence encoding the chimeric mRNA can comprise a nucleic acid sequence comprising about 75% or more, at least about 85% or more, at least about 95% or more, or 100% sequence identity with a nucleic acid sequence of SEQ ID NO: 38.

In some aspects, the chimeric mRNA comprises a nucleic acid sequence comprising four binding sites of a Bruno-like RNA-binding protein. The nucleic acid sequence encoding the chimeric mRNA can comprise a nucleic acid sequence comprising about 75% or more, at least about 85% or more, at least about 95% or more, or 100% sequence identity with a nucleic acid sequence of SEQ ID NO: 45.

In some aspects, the chimeric mRNA can comprise a nucleic acid sequence comprising a nucleic acid sequence comprising the 3′ UTR of a plant SOC1 mRNA. The nucleic acid sequence encoding the chimeric mRNA can comprise a nucleic acid sequence comprising about 75% or more, at least about 85% or more, at least about 95% or more, or 100% sequence identity with a nucleic acid sequence of SEQ ID NO: 47.

Another aspect of the instant disclosure encompasses two or more expression constructs encoding an engineered system for tethering a protein of interest to a target RNA molecule. The two or more expression constructs comprise an expression construct for expressing an engineered protein comprising an RNA-binding polypeptide fused to a protein or polypeptide of interest; and an expression construct for expressing the target RNA molecule, the expression construct comprising a promoter operably linked to a nucleic acid sequence encoding the target RNA molecule. The nucleic acid expression construct for expressing the engineered protein and the target RNA molecule can be as described above.

In some aspects, the nucleic acid expression construct for expressing the target RNA molecules expresses an RNA molecules expressing a Cas9 protein of a CRISPR/Cas programmable nucleic acid modification system. The two or more expression constructs can further comprise an expression construct for expressing a guide RNA (gRNA) of the CRISPR/Cas programmable nucleic acid modification system, the expression construct comprising a promoter operably linked to a nucleic acid sequence encoding the gRNA. In some aspects, the gRNA comprises a sequence complementary to a target sequence within a nucleotide sequence encoding an ADH1 protein.

An additional aspect of the instant disclosure encompasses an expression vector for tethering a protein of interest to an RNA molecule. The expression vector comprises a first nucleic acid sequence encoding the RNA-binding polypeptide, and a second nucleic acid sequence comprising a cloning sequence upstream or downstream of the first nucleic acid sequence, whereby a third nucleic acid sequence encoding the protein or polypeptide of interest cloned into the cloning sequence generates the nucleic acid sequence encoding the engineered protein. The expression vector can comprise a UBQ10 promoter operably linked to a nucleic acid sequence encoding an engineered protein comprising a flag tag fused to the N-terminus of an RNA-binding polypeptide comprising one or more RRMs of a Bruno-like protein, a nucleic acid sequence comprising a cloning sequence downstream of the nucleic acid sequence encoding the RNA-binding polypeptide, and an OCS terminator. In some aspects, the expression construct comprises a nucleic acid sequence comprising about 75% or more, at least about 85% or more, at least about 95% or more, or 100% sequence identity with a nucleic acid sequence of SEQ ID NO: 18.

Another aspect of the instant disclosure encompasses an engineered protein for tethering a protein of interest to a target RNA molecule. The engineered protein comprises an RNA-binding polypeptide fused to a protein or polypeptide of interest, wherein the RNA-binding polypeptide comprises an RNA-binding domain of an RNA-binding protein, wherein the RNA-binding domain recognizes and specifically binds an RNA recognition sequence in the target RNA molecule to thereby tether the protein of interest to the target RNA molecule. The engineered protein is encoded by an expression construct as described herein above.

Yet another aspect of the instant disclosure encompasses a cell comprising one or more expression constructs for expressing an engineered protein for tethering a protein of interest to a target RNA molecule, two or more expression constructs encoding an engineered system for tethering a protein of interest to a target RNA molecule, an expression vector expression vector for tethering a protein of interest to an RNA molecule, or any combination thereof. The cell can be a eukaryotic cell such as a plant cell.

One aspect of the instant disclosure encompasses a method of determining the function of a protein of interest or an RNA molecule in RNA-protein interactions. The method comprises the steps of (a) providing or having provided one or more expression constructs for expressing an engineered protein for tethering a protein of interest to a target RNA molecule, two or more nucleic acid expression constructs encoding an engineered system for tethering a protein of interest to a target RNA molecule, an expression vector for tethering a protein of interest to an RNA molecule, a cell comprising one or more expression construct thereof, or any combination thereof; (b) introducing the one or more expression constructs into a cell, culturing the cell under conditions suitable for expressing the engineered protein, wherein the RNA-binding polypeptide comprises an RNA-binding domain of an RNA-binding protein and wherein the RNA-binding domain recognizes and specifically binds an RNA recognition sequence in the target RNA molecule to thereby tether the protein of interest to the target RNA molecule; and (c) investigating one or more phenotypes resulting from tethering the protein of interest to the target RNA molecule in the cell to thereby determine the function of the protein of interest or the target RNA molecule in RNA-protein interaction.

Another aspect of the instant disclosure encompasses a method of tethering a protein of interest to a target RNA molecule. The method comprises expressing an engineered protein using a nucleic acid expression construct for tethering the protein of interest to the target RNA molecule encoded by expression constructs for expressing an engineered protein for tethering a protein of interest to a target RNA molecule in a cell comprising the target RNA molecule. The engineered protein and expression construct encoding the engineered protein can be as described herein above. The method can further comprise expressing the target RNA molecule using a nucleic acid expression construct for expressing the target RNA molecule.

Another aspect of the instant disclosure encompasses a method of identifying one or more RNA-binding proteins that bind a target RNA. The method comprises the steps of (a) expressing an engineered protein for tethering a protein of interest to a target RNA molecule using a nucleic acid expression construct for tethering the protein of interest to the target RNA molecule described above in a cell comprising the target RNA molecule; isolating the target RNA molecule or the engineered protein of step (a) and proteins bound to the target RNA molecule or the engineered protein from the cell; and identifying the proteins bound to the target RNA molecule from the isolated target RNA molecule or the protein of interest of step (b).

An additional aspect of the instant disclosure encompasses a method of modifying a function of a target RNA molecule, the method comprising tethering a protein of interest to a target RNA molecule using a method described above, wherein tethering the protein of interest modifies the function of the target RNA molecule. In some aspects, the protein of interest is a de-adenylase protein, the target RNA molecule is an mRNA, and the tethered de-adenylase protein reduces the stability of the mRNA. In some aspects, the protein of interest is a translation enhancer protein, the target RNA molecule is an mRNA, and the tethered translation enhancer protein increases translation of the mRNA.

One aspect of the instant disclosure encompasses kits comprising one or more expression constructs for expressing an engineered protein for tethering a protein of interest to a target RNA molecule, two or more expression constructs encoding an engineered system for tethering a protein of interest to a target RNA molecule, one or more expression vectors for tethering a protein of interest to an RNA molecule, or any combination thereof. The kits can also comprise cells comprising the expression constructs and vectors, an engineered protein and/or system for tethering a protein of interest to a target RNA molecule or any combination thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is a diagrammatic depiction of the Arabidopsis BRN1 protein which comprises three RRM domains (1-3) that bind the SOC1 mRNA 3′UTR. Only two RRM domains (1-2) are necessary for Bruno-like proteins to bind their targets (Snee et al., 2008). The BRN1 protein inhibits SOC1 translation, and this is thought to be mediated via the protein region between RRM 2 and 3. A FLAG epitope-tagged (asterisk) truncated BRN1 protein called FLAG-BD was generated with only RRM domains 1 and 2 (FLAG-BD, bottom).

FIG. 1B are protein electrophoresis gels and Westerns from an immunoprecipitation (IP) using the FLAG epitope in plants with and without FLAG-BD. The three wt Col and FLAG-BD samples were biological replicates. PEP is an unrelated protein used as a loading control. FT=fraction unbound to the FLAG antibody. Arrowheads mark the predicted size of the protein detected.

FIG. 1C depicts the FLAG-IP followed by RNA extraction (FLAG-RIP) and qRT-PCR of samples from FIG. 1B. AT2G20610 is an unrelated gene used as a negative control. Each biological replicate is shown as a point. The bar represents the average and error bars represent the standard deviation between three biological replicates. P-value is calculated by using an unpaired t-test with Welch's correction. The RIP experiment was repeated twice (Rep 1/Rep 2) using distinct biological replicate plants)

FIG. 1D depicts that FLAG-BD interacts with other mRNAs in addition to SOC1. Extended analysis of the experiment from FIG. 1C for three additional mRNAs that have the identical 7-nt BRN1 binding site sequence. Each biological replicate is shown as a point. The bar represents the average and error bars represent the standard deviation between three biological replicates.

FIG. 2A is a plot of flowering time measured as the number of leaves generated at the time the first flower opens. Gray points are individual plants, and the box plots represent the 25th and 75th percentiles of the sample population, with the center bar representing the median and whiskers at the 10th and 90th percentile. P-value is calculated by using unpaired t-test. ns=not statistically significant.

FIG. 2B depicts the qRT-PCR of SOC1 mRNA levels in plants with and without FLAG-BD. Three biological replicates for each genotype were used (shown as red points), the height of the bar represents their average and the error bars represent the standard deviation. P-value is calculated by using unpaired t-test with Welch's correction.

FIG. 2C depicts a Western blot displaying SOC1 protein levels between biological replicates with and without FLAG-BD.

FIG. 2D depicts SOC1 protein quantification from the Western blot in FIG. 2C. Biological replicate data points are shown as blue points, the height of the bar represents their average and error bars represent the standard deviation. P-value is calculated by using unpaired t-test with Welch's correction.

FIG. 2E depicts the accumulation of siRNAs from wt Col and FLAG-BD lines. TAS3 is a trans-acting siRNA producing locus shown as a positive control for siRNA accumulation. AT2G20610 is an unrelated gene without small RNA production used as a negative control. Two biological replicates are shown as points, and their average is the height of the bar. RPM=reads per million sequenced small RNAs.

FIG. 3A depicts a Volcano plot of anti-FLAG immunoprecipitation followed by Mass Spectrometry (IP-MS) of four biological replicates (Rep 1-4). The x-axis shows the log 2 of fold change between each FLAG-IP sample and the mock-IP control, and the y-axis depicts the p-value of Fisher exact test in a negative log scale. The gray shaded region represents the region of statistical significance (fold change ≥2, p≤0.05), while the pink shaded region represents proteins that accumulated in the FLAG-IP but did not accumulate in the mock-IP, making their enrichment value infinite. The green point indicates the bait protein BRN1. Red points indicate the significantly enriched proteins. Blue points indicate the proteins significantly enriched in at least 2 of the 4 biological replicates.

FIG. 3B depicts a stacked bar graph showing the RNA-interaction annotation of the 20 proteins enriched in at least 2 of the 4 biological replicates (FLAG-BD enriched, blue points from part A). These are compared to the proteins identified in the mock-IP (FLAG-BD mock), the annotation of the entire Arabidopsis genome, or the genes annotated as expressed in rosette or cauline leaves. Bold indicates the percent of genes, with the number of genes below in parentheses. The hypergeometric test results are shown as fold enrichments (top number) and p-value in parentheses.

FIG. 4A is a diagrammatic depiction of the translational fusion between FLAG-BD and the CAF1a de-adenylase that generates the BD+D protein.

FIG. 4B is a plot showing the flowering time of BD+D plants two (T2) and three (T3) generations after transformation. +T plants inherited the BD+D transgene, −T plants are siblings that did not inherit the transgene. Box plots and statistics are the same as in FIG. 2A.

FIG. 4C depicts a plot showing the reduced expression of the BD+D transgene. qRT-PCR of the relative mRNA accumulation of the FLAG-BD and BD+D transgenes. AT2G20610 is a constitutively-expressed control gene. At least three biological replicates for each genotype were used (shown as points), the height of the bar represents their average and the error bars represent the standard deviation. The transgene structure and position of the RT-PCR primers is shown above.

FIG. 4D depicts the qRT-PCR of SOC1 polyadenylated mRNA. Three biological replicates of each genotype are shown as red points. Bar height, error bars and statistics are the same as FIG. 2B.

FIG. 4E depicts the qRT-PCR of SOC1 nascent transcripts (unspliced and not polyadenylated). Three biological replicates of each genotype are shown as red points. Bar height, error bars and statistics are the same as FIG. 2B.

FIG. 4F depicts results of an ePAT assay used to determine the poly(A) tail length of the SOC1 mRNA. n=the number of clones Sanger sequenced. TVN is a control where the reverse transcription primer is anchored at the most 3′ nucleotide before the poly(A) tail begins. Box plot organization is the same as FIG. 2A. P-value is calculated by using an unpaired t-test with Welch's correction.

FIG. 4G depicts the quantification of SOC1 protein accumulation in the BD+D line. Individual biological replicates are down as blue points. Bar height, error bars and statistics are the same as FIG. 2B.

FIG. 4H depicts a photograph of a western blot (top panel) showing SOC1 protein accumulation in BD+D plants. Western blot displaying SOC1 protein levels with and without the BD+D transgene. The three wt Col and BD+D samples are biological replicates. PEP is an unrelated protein used as a loading control. Arrowheads mark the predicted size of the protein detected. Quantification of this Western blot is shown in FIG. 4G. This construct was designed to enhance RNA translation.

FIG. 5A is a diagrammatic depiction of the translational fusion between FLAG-BD and the RPS6 ribosomal protein generates the BD+R protein.

FIG. 5B depicts a plot showing the flowering time of T2 BD+R plants grown side-by-side with wt Col. +T plants inherited the BD+D transgene, −T plants are siblings that did not inherit the transgene. Box plots and statistics are the same as in FIG. 2A.

FIG. 5C shows representative images of T2 BD+R (+T) plants grown side-by-side with wt Col taken 24 days after germination.

FIG. 5D is a photograph of a Western blot of SOC1 protein accumulation in seven biological replicates of early-flowering plants of the BD+R line compared to wt Col. PEP is an unrelated protein used as a loading control.

FIG. 5E is a plot showing the quantification of SOC1 protein accumulation in the BD+R line from FIG. 5D. Individual biological replicates are down as blue points. Bar height, error bars and statistics are the same as FIG. 2B.

FIG. 5F is a qRT-PCR plot of SOC1 polyadenylated mRNA. Three biological replicates of each genotype are shown as red points. Bar height, error bars and statistics are the same as FIG. 2B.

FIG. 5G is a qRT-PCR plot of SOC1 nascent transcripts (unspliced and not polyadenylated). Three biological replicates of each genotype are shown as red points. Bar height, error bars and statistics are the same as FIG. 2B.

FIG. 6A depicts five transgenes generated to test moving the BRN1 binding site to a new RNA transcript. The RNA transcript encodes the Cas9 protein. The top construct has no 3′ UTR (no BRN1 binding site) as a negative control, the next has 1×7 nt BRN1 binding site, the third has 4×BRN1 binding sites separated by 20 nt, the fourth has the full SOC1 3′UTR (with naturally-occurring 1×BRN1 binding site), and the fifth has the same SOC1 3′UTR but with the 7 nt BRN1 binding site removed.

FIG. 6B depicts Cas9 protein levels measured by quantitative Western blots and normalized to Actin protein levels. Each dot represents a T1 biological replicate.

FIG. 6C depicts Cas9-mediated mutation rate in T1 individual plants. Total number of individual tested (n) is listed on the top of each transgenic line. Percentage of individual with Cas9-mediated mutation shows in each bar.

### DETAILED DESCRIPTION

The instant disclosure is based in part on the surprising discovery that RNA-binding domains of RNA-binding proteins can be used to engineer proteins capable of routinely and specifically tethering a protein of interest to an RNA molecule in a cell. The engineered proteins do not require the RNA molecule to be altered or engineered and can be used to tether a protein of interest to any target RNA molecule.

The engineered proteins can be used to drive the RNA molecule into pathways of post-transcriptional modulation to control gene expression and to investigate the function of proteins that interact or are suspected of interacting with RNA in vivo, an important distinction when compared to currently available technologies. For instance, the engineered proteins can be used increase or reduce production of a protein encoded by an mRNA molecule. In addition, the engineered proteins can be used to investigate and identify previously unidentified RNA-binding proteins that bind and regulate the function of a target RNA molecule. Importantly, the engineered proteins can be used to tether proteins in plants, a feat that was previously considered impossible.

The instant disclosure is also directed to expression constructs for expressing the engineered proteins, an engineered system for tethering a protein of interest to a target RNA molecule, expression vectors that facilitate generation of engineered proteins comprising any protein of interest linked to an RNA-binding polypeptide, and methods of using the engineered proteins and expression constructs and vectors. This system is useful to boost protein production from any user-defined engineered gene.

### I. Expression Constructs

One aspect of the instant disclosure encompasses an expression construct for expressing an engineered protein for tethering a protein of interest to a target ribonucleic acid (RNA) molecule. The expression construct comprises a promoter operably linked to a nucleic acid sequence encoding the engineered protein. The nucleic acid sequence encoding the engineered protein comprises a first nucleic acid sequence encoding the RNA-binding polypeptide and a second nucleic acid sequence encoding the protein or polypeptide of interest upstream or downstream of the first nucleic acid sequence. Alternatively, the expression construct can comprise a first nucleic acid sequence encoding the RNA-binding polypeptide, and a second nucleic acid sequence comprising a cloning sequence upstream or downstream of the first nucleic acid sequence, whereby a third nucleic acid sequence encoding the protein or polypeptide of interest cloned into the cloning sequence generates the nucleic acid sequence encoding the engineered protein.

The RNA-binding polypeptide comprises an RNA-binding domain of an RNA-binding protein, wherein the RNA-binding domain recognizes and specifically binds an RNA recognition sequence in the target RNA molecule, to thereby tether the protein of interest to the target RNA molecule.

### (a) RNA-Binding Polypeptide

The engineered protein comprises an RNA-binding polypeptide linked to a protein or polypeptide of interest. The RNA-binding polypeptide can comprise an RNA-binding domain of an RNA-binding protein. The RNA-binding domain can recognize and specifically bind an RNA recognition sequence in the target RNA molecule. By binding the RNA recognition sequence in the target RNA molecule, the binding protein tethers the fused protein or polypeptide of interest to the target RNA molecule.

The one or more RNA-binding domains can be selected from an RNA recognition motif (RRM), a helix-turn-helix domain, a leucine zipper domain, a winged helix domain, a winged helix-turn-helix domain, a helix-loop-helix domain, an HMG-box domain, a White-Opaque Regulator 3 (Wor3) domain, an OB-fold domain, an immunoglobulin fold, a B3 domain, a TAL effector, a dsRNA-binding domain, a zinc finger domain, zinc knuckles, a KH domain, and any combination thereof.

It will be recognized that RNA-binding domains of RNA-binding proteins can be engineered or “programmed” to recognize and specifically bind any RNA sequence on a target RNA molecule. Accordingly, an aspect of the instant disclosure comprises an engineered RNA-binding protein comprising an engineered RNA-binding domain of an RNA-binding protein, wherein the RNA-binding domain is programmed to recognize and specifically bind a nucleic acid sequence on any RNA molecule. Accordingly, an engineered RNA-binding protein of the instant disclosure comprising an engineered RNA-binding domain of an RNA-binding protein can be programmed to target any RNA molecule and tether a protein of interest to the target RNA molecule. As explained further below in Section I(b) herein below, the target RNA molecule can be an endogenous wild type RNA molecule or can be an exogenously expressed RNA molecule, either of which can comprise an introduced RNA recognition sequence or can comprise an RNA sequence of the target RNA molecule that can be recognized by an engineered RNA-binding protein.

In some aspects, the RNA-binding domain comprises one or more RRMs of an RNA-binding protein, also known as ribonucleoprotein (RNP) motifs or RNP-type RNA-binding domains (RBD). RRMs are the most common RNA-binding domains and comprise a small protein domain of 75-100 (about 90) amino acids whose amino acid sequence is conserved from yeast to man. Proteins comprising RRMs (RRM proteins) exhibit highly specific recognition of their RNA targets by recognizing and binding specific RNA sequences (RRM recognition sequences) on an RNA molecule. RRM proteins play a role in almost all aspects of RNA processing and transport, including RNA processing, splicing, translation regulation, transport, RNA export, quality control, storage, and RNA stability. RRMs can be present as a single copy or in multiple copies in RNA-binding proteins, wherein some proteins require multiple copies of the RRM for tight and specific RNA binding. The spliceosomal U1 70 kDa protein and hnRNP C1 each contain only a single copy of RRM, whereas the 65 kDa subunit of the U2 auxiliary factor (U2AF) contains two copies, ELAV protein contains three copies, and the poly(A) (polyadenylate)-binding protein contains four tandem copies. However, when an RRM protein comprises multiple RRMs, not all RRMs are required for RNA binding, and hence some may have still other functions, such as mediating protein-protein interaction. The most highly conserved sequences within RRMs are the ribonucleoprotein 1 (RNP1) and RNP2 motifs that are signature sequences for the RRM proteins. The RNP1 and RNP2 consensus sequences are (R/K)-G-(F/Y)-(G/A)-(F/Y)—V-X-(F/Y) (SEQ ID NO: 28) and (L/I)-(F/Y)-(V/I)-X-(N/G)-L (SEQ ID NO: 29), respectively. There are at least 196 proteins in Arabidopsis that have RRM motifs. They include Poly(A)-binding proteins (PABPs), Ser/Arg (SR) and spliceosomal RRM-containing proteins (SR proteins), oligouridylate binding proteins (UBP1, RBP45,UBA1), Chloroplast RRM-containing proteins (cpRNPs), and glycine-rich RNA-binding proteins (GR-RBPs) among others.

An RRM of an RNA-binding polypeptide can be from any protein comprising one or more RRMs. Further, any of the one or more RRMs of a binding protein can be wild type or can be modified for modified specificity in recognition and strength of binding of RNA recognition sequences on a target RNA molecule. RNA-binding polypeptides of the instant disclosure can comprise 1, 2, 3, 4, 5, 6, 7, 8, 9, 10, or more RRMs. In some aspects, the RNA-binding polypeptide comprises one RRM. In other aspects, the RNA-binding polypeptide comprises two RRMs. In yet other aspects, the RNA-binding polypeptide comprises three RRMs. In some aspects, the RNA-binding polypeptide comprises four RRMs. When the RNA-binding polypeptide comprises more than one RRM, the RRMs can be multiple identical copies of an RRM, more than one non-identical RRM, or any combination of multiple copies of an RRM and more than one RRM.

In some aspects, the RNA-binding polypeptide comprises an amino acid sequence comprising one or more RRMs of a Bruno or Bruno-like RNA-binding protein (Bruno-like RNA-binding protein or Bruno-like protein). Bruno-like proteins are a family of single strand RNA-binding polypeptides that regulate the development of organisms. Bruno-like proteins are expressed in various organisms, including humans, Drosophila, and plants. Six Bruno-like proteins have been identified in humans, at least three in Drosophila melanogaster, six in Xenopus laevis, and one in Caenorhabditis elegans. Three Bruno-like proteins have been identified in Arabidopsis, including FCA, AtBRN2, and AtBRN1.

In some aspects, the RNA-binding polypeptide comprises an amino acid sequence comprising one or more RRMs of a plant BRN1 protein. In some aspects, the RNA-binding polypeptide comprises an amino acid sequence comprising one or more RRMs of an Arabidopsis sp BRN1 protein. The BRN1 protein can comprise an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 47. In some aspects, the BRN1 protein comprises about 75% or more, at least about 85% or more, at least about 95% or more, or 100% sequence identity with an amino acid sequence of SEQ ID NO: 30. In some aspects, the BRN1 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 31 or SEQ ID NO: 32. In some aspects, the BRN1 protein is encoded by a nucleic acid sequence comprising about 75% or more, at least about 85% or more, at least about 95% or more, or 100% sequence identity with a nucleic acid sequence of SEQ ID NO: 31 or SEQ ID NO: 32.

The BRN1 RNA-binding protein comprises three RRMs, termed RRM A, RRM B, and RRM C. In some aspects, the RNA-binding polypeptide comprises an amino acid sequence comprising RRM A of a BRN1 protein. In other aspects, the RNA-binding polypeptide comprises an amino acid sequence comprising RRM B of a BRN1 protein. In yet other aspects, the RNA-binding polypeptide comprises an amino acid sequence comprising RRM C of a BRN1 protein. In some aspects, the RNA-binding polypeptide comprises an amino acid sequence comprising RRM A and RRM C of a BRN1 protein. In additional aspects, the RNA-binding polypeptide comprises an amino acid sequence comprising RRM B and RRM C of a BRN1 protein. In some aspects, the RNA-binding polypeptide comprises an amino acid sequence comprising RRM A and RRM B of a BRN1 protein. In some aspects, the RNA-binding polypeptide comprises an amino acid sequence comprising RRM A and RRM B of a BRN1 protein, and an amino acid sequence comprising RRM C of the BRN1 protein is absent in the RNA-binding domain polypeptide.

In some aspects, the RRM A and RRM B of BRN1 comprise an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 97. In some aspects, the RRM A and RRM B of BRN1 comprise an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 97.

In some aspects, the RNA-binding polypeptide comprises an amino acid sequence comprising the RRMs A-B of a Bruno-like RNA-binding polypeptide, and the RNA-binding polypeptide comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 33. In some aspects, the RNA-binding polypeptide comprises an amino acid sequence comprising the RRMs A-B of a Bruno-like RNA-binding polypeptide, and the RNA-binding polypeptide comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 33. In some aspects, the RNA-binding polypeptide is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 34. In some aspects, the RNA-binding polypeptide comprising RRMs A-B of a Bruno-like RNA-binding protein is encoded by a nucleic acid sequence comprising about 75% or more, at least about 85% or more, at least about 95% or more, or 100% sequence identity with a nucleic acid sequence of SEQ ID NO: 34.

An RNA-binding polypeptide comprising one or more RRMs of a Bruno-like RNA-binding protein recognizes and specifically binds an RNA recognition sequence comprising one or more Bruno-like protein binding site on the target RNA molecule. When the Bruno-like RNA-binding protein is BRN1, the RNA-binding polypeptide recognizes and specifically binds an RNA sequence comprising one or more BRN1 binding sites on the target RNA molecule. The RNA sequence comprising the one or more BRN1 binding sites can comprise about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 5. In some aspects, the RNA sequence comprising the one or more BRN1 binding sites comprises about 75% or more, at least about 85% or more, at least about 95% or more, or 100% sequence identity with a nucleic acid sequence of SEQ ID NO: 5. In some aspects, the RNA sequence comprising the one or more BRN1 binding sites comprises a nucleic acid sequence of SEQ ID NO: 5.

### (b) Protein or Polypeptide of Interest

An engineered protein of the instant disclosure comprises an RNA-binding polypeptide linked to a protein or polypeptide of interest. The RNA-binding polypeptide can be as described in Section I(a) herein above.

In some aspects, the protein or polypeptide of interest is fused to the RNA-binding polypeptide. The protein or polypeptide of interest can be fused to the RNA-binding polypeptide at the N terminus, the C terminus, or internal to the RNA-binding polypeptide. The RNA-binding protein can further comprise a linker linking the RNA-binding polypeptide and the protein of interest. Linkers can be as described below in Section I(d)(B).

In some aspects, the RNA-binding protein comprises a polypeptide of interest fused to the RNA-binding polypeptide. The polypeptide of interest can indirectly attach the protein of interest to the RNA-binding protein by binding the protein of interest. For instance, the polypeptide of interest can comprise an affinity polypeptide that binds an epitope on the protein of interest, thereby attaching the protein of interest to the RNA-binding protein. Non-limiting examples of affinity polypeptides include antibodies, antibody fragments, peptides, aptamers, peptidomimetics, a ligand, a ligand fragment, a receptor, an epitope or purification tag, or a receptor fragment, alone or in combination.

The protein of interest can be a protein known to interact with RNA, is suspected of interacting with RNA, or can also be of unknown function. The protein can be a ribonucleoprotein that directly binds RNA or can be a protein that is indirectly associated with RNA through interactions with one or more RNA-binding polypeptides in a ribonucleo-protein (RNP) complex. The protein of interest can also be a protein that does not bind RNA but can be associated with RNA in an RNP through other molecular interactions with components of the RNP. The protein of interest can be a protein involved in post-transcriptional regulation of expression and can be tethered to an RNA molecule to regulate post-transcriptional expression of the RNA molecule. Alternatively, a protein of interest can be a protein that plays no function in post-transcription gene expression. For instance, the protein of interest can be a reporter protein such as a visual reporter, and the reporter can be tethered to the RNA molecule to observe post-transcriptional events of an RNA molecule. The protein of interest can be an endogenous protein or can be an exogenously expressed protein.

Further, the engineered protein can comprise more than one protein or polypeptide of interest linked to the RNA-binding polypeptide. For instance, more than one protein of interest can be fused in line with the RNA-binding polypeptide. Alternatively, when the engineered protein comprises a polypeptide of interest, the polypeptide of interest can comprise one or more affinity polypeptides to attach one or more proteins of interest to the RNA-binding polypeptide. The engineered protein can comprise more than one affinity polypeptide, each of which can specifically bind an epitope on a protein of interest to thereby attach the proteins of interest to the RNA-binding polypeptide. Further, an affinity polypeptide can specifically bind more than one protein of interest to thereby attach the proteins of interest to the RNA-binding polypeptide.

In some aspects, the protein of interest comprises a protein that functions in post-transcriptional regulation of expression. For instance, the protein of interest can be a protein that can modulate stability, RNA decay, RNA processing, alternative splicing, polyadenylation of mRNAs, localization of an RNA molecule in a cell, or proteins that perform quality control, RNA editing, transport, storage, and export.

In some aspects, the protein of interest comprises a protein selected from an RNA de-adenylase protein, an RNA processing protein, an RNA translation regulation protein, an RNA decay protein, an RNA localization protein, and any combination thereof. Non-limiting examples of proteins of interest include CAF1, RDR6, RDR3, SGS3, SDE5, SDE3, AGO1, AGO7, AGO8, RDR3, RDR4, RSP6, EIF6A, and any combination thereof.

In some aspects, the protein of interest is an RNA de-adenylase protein. In one aspect, the de-adenylase is a CAF1 protein. In one aspect, the protein of interest is an Arabidopsis CAF1 protein. In some aspects, the RNA de-adenylase protein is an Arabidopsis de-adenylase protein comprising an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6. In some aspects, the CAF1 protein comprises an amino acid sequence comprising about 75% or more, at least about 85% or more, at least about 95% or more, or 100% sequence identity with an amino acid sequence of SEQ ID NO: 6. In some aspects, the CAF1 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 7. In some aspects, the CAF1 protein is encoded by a nucleic acid sequence comprising about 75% or more, at least about 85% or more, at least about 95% or more, or 100% sequence identity with a nucleic acid sequence of SEQ ID NO: 7.

In some aspects, the protein of interest comprises an RNA decay protein. In some aspects, the RNA decay protein is an RNAi factor that has a known function in mRNA degradation via RNA interference (RNAi). Non-limiting examples of proteins that function in mRNA degradation via RNAi include the A. thaliana RDR3, RDR6, SDE3, SDE5, SGS3, RDR4, RDR5, AGO7, AGO8 and AGO1 proteins. Accordingly, in some aspects, the protein of interest comprises an A. thaliana protein selected from RDR3, RDR6, SDE3, SDE5, SGS3, RDR4, RDR5, AGO1, AGO7, AGO8 and any combination thereof. In some aspects, the RNAi factor is fused to the RNA-binding polypeptide. In one aspect, the RNAi factor is fused to the C terminus of the RNA-binding polypeptide.

In some aspects, the protein of interest is a protein that can improve expression of a protein encoded by an mRNA. For instance, the protein of interest can be a translation enhancer protein and/or a protein that stabilizes the RNA, thereby resulting in improved expression of the protein encoded by the mRNA. Non-limiting examples of proteins that function in mRNA stabilization and the promotion of translation include the A. thaliana RSP6 and EIF6A.

In some aspects, the protein of interest is a translation enhancer protein. In some aspects, the translation enhancer is an RPS6 protein. In some aspects, the RPS6 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 11. In some aspects, the RPS6 protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 11. In some aspects, the RPS6 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 12. In some aspects, the RPS6 protein is encoded by a nucleic acid sequence comprising about 75% or more, at least about 85% or more, at least about 95% or more, or 100% sequence identity with a nucleic acid sequence of SEQ ID NO: 12.

### (c) Target RNA Molecules

Engineered proteins of the instant disclosure tether a protein of interest to an RNA molecule by recognizing and specifically binding an RNA recognition sequence in the target RNA molecule, to thereby tether the protein of interest to the target RNA molecule. Proteins of interest can be as described in Section I(b) herein above, and RNA-binding polypeptides can be as described in Section I(a) herein above.

A target RNA molecule can be a protein coding mRNA or a non-coding RNA. Non-coding RNAs (“ncRNA”) can be encoded by their own genes (RNA genes), but can also derive from protein-coding genes or mRNA introns. Non-limiting examples of non-coding RNAs include transfer RNA (tRNA), ribosomal RNA (rRNA), miRNAs, long non-coding RNAs (lncRNA), long non-translated RNAs (IntRNA), trans-acting siRNAs (tasiRNAs), antisense mRNAs, enhancer RNAs, introns, snRNAs, snoRNAs, and ribozymes. RNA molecules can also be viral genomes and viral transcripts. For instance, tethering a protein to a viral RNA to recruit the viral RNA into an RNA degradation pathway or to decrease translation of the viral RNA to control viral infections. In some aspects, the target RNA molecule is an mRNA.

The RNA recognition sequence can be at any location in the RNA molecule accessible to the RNA-binding polypeptide. For instance, when the RNA molecule is an mRNA, the recognition sequence can be in a non-coding region of the RNA such as in the 5′ or 3′ UTR or an intron or can be internal to the coding sequence. In some aspects, an RNA recognition sequence is in a non-coding region of an mRNA. In some aspects, an RNA recognition sequence is in the 5′ UTR of an mRNA. In some aspects, RNA recognition sequences are in the 3′ UTR of an mRNA.

The target RNA molecule can be an endogenous RNA molecule or can be an exogenously expressed RNA molecule. The target RNA molecule can be a wild type RNA molecule that comprises an RNA recognition sequence. Alternatively, an RNA recognition sequence can be cloned into an RNA molecule to generate an engineered target RNA molecule comprising an RNA recognition sequence to thereby induce tethering of a protein of interest to the RNA molecule. Further, a target RNA molecule can be any RNA molecule, and the engineered RNA-binding protein of the instant disclosure can comprise an engineered RNA-binding domain of an RNA-binding protein, wherein the RNA-binding domain is programmed to recognize and specifically bind a nucleic acid sequence on the RNA molecule (See Section I(a) herein above).

The RNA molecule can comprise more than one RNA recognition sequence. For instance, the RNA molecule can comprise 1, 2, 3, 4, 5, 6, 7, 8, 9, 10, or more RNA recognition sequences. Multiple RNA recognition sequences can strengthen the binding of the RNA-binding protein to the target RNA molecule or can tether more than one protein to the RNA molecule. The more than one recognition sequence can be identical copies of the RNA recognition sequence to tether engineered proteins having binding specificity to the recognition sequence or can be non-identical recognition sequences to tether engineered proteins having binding specificity to the non-identical recognition sequences.

In some aspects, the target RNA molecule comprises one or more RNA recognition sequence specifically recognized by a Bruno-like RNA-binding protein. The RNA recognition sequence specifically recognized by a Bruno-like RNA-binding protein can comprise one or more Bruno-like protein binding site.

In some aspects, the RNA molecule comprises a messenger RNA (mRNA) molecule, and the mRNA molecule comprises one or more binding sites of a Bruno-like RNA-binding protein in a non-coding region of the mRNA molecule, internal to the coding sequence, or any combination thereof. In some aspects, the mRNA molecule comprises one or more RNA-binding sites of a Bruno-like RNA-binding protein in an untranslated region (UTR) of the mRNA. In one aspect, the RNA molecule comprises one or more binding sites of a Bruno-like RNA-binding protein in a 3′ untranslated region (3′ UTR) of the mRNA.

In some aspects, the nucleic acid sequence comprising the one or more binding sites of a Bruno-like RNA-binding protein comprises a nucleic acid sequence comprising the 3′ UTR of a plant SOC1 mRNA. In some aspects, the 3′ UTR of a plant SOC1 mRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 37. In some aspects, the 3′ UTR of a plant SOC1 mRNA comprises a nucleic acid sequence comprising about 75% or more, at least about 85% or more, at least about 95% or more, or 100% sequence identity with a nucleic acid sequence of SEQ ID NO: 37.

In some aspects, the mRNA molecule comprises a plant SOC1 mRNA. In some aspects, the SOC1 mRNA comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 35. In some aspects, the SOC1 mRNA comprises a nucleic acid sequence comprising about 75% or more, at least about 85% or more, at least about 95% or more, or 100% sequence identity with a nucleic acid sequence of SEQ ID NO: 35. In some aspects, the SOC1 mRNA is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 36. In some aspects, the SOC1 mRNA is encoded by a nucleic acid sequence comprising about 75% or more, at least about 85% or more, at least about 95% or more, or 100% sequence identity with a nucleic acid sequence of SEQ ID NO: 36.

In some aspects, the target RNA molecule is a chimeric mRNA comprising a nucleic acid sequence comprising one or more binding sites of a Bruno-like RNA-binding protein cloned in a 3′ UTR of the mRNA. In some aspects, the chimeric mRNA encodes a Cas9 protein of a CRISPR/Cas programmable nucleic acid modification system. In some aspects, the chimeric mRNA encodes Cas9 and comprises one or more binding sites of a Bruno-like RNA-binding protein.

In some aspects, the chimeric mRNA encodes Cas9 and comprises one binding site of a Bruno-like RNA-binding protein. In some aspects, the chimeric mRNA is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 38. In some aspects, the chimeric mRNA is encoded by a nucleic acid sequence comprising about 75% or more, at least about 85% or more, at least about 95% or more, or 100% sequence identity with a nucleic acid sequence of SEQ ID NO: 38.

In some aspects, the chimeric mRNA encodes Cas9 and comprises four binding sites of a Bruno-like RNA-binding protein. In some aspects, the chimeric mRNA is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 45. In some aspects, the chimeric mRNA is encoded by a nucleic acid sequence comprising about 75% or more, at least about 85% or more, at least about 95% or more, or 100% sequence identity with a nucleic acid sequence of SEQ ID NO: 45.

In some aspects, the chimeric mRNA encodes Cas9 and comprises a nucleic acid sequence comprising the 3′ UTR of the SOC1 mRNA. In some aspects, the chimeric mRNA is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 47. In some aspects, the chimeric mRNA is encoded by a nucleic acid sequence comprising about 75% or more, at least about 85% or more, at least about 95% or more, or 100% sequence identity with a nucleic acid sequence of SEQ ID NO: 47.

### (d) Other Components

An engineered RNA-binding polypeptide encoded by an expression construct of the instant disclosure can further comprise additional amino acid sequences to enhance or supplement the function of the protein. Non-limiting additional components include affinity tags and linkers.

### A. Affinity Tags

In some aspects, the RNA-binding protein further comprises an amino acid sequence comprising an affinity tag fused to a second terminus of the RNA-binding polypeptide. An affinity tag is a peptide sequence that can be used in many different assays that require recognition by an epitope. Non-limiting examples of assays that can be performed using affinity tags include cellular localization studies by immunofluorescence, immunoprecipitation, or detection by SDS PAGE protein electrophoresis and Western blotting. Affinity tags are fused to proteins to facilitate purification of the protein from a crude biological source, using an affinity technique. Any suitable affinity tag known in the art can be used as desired. Non-limiting examples of suitable affinity tags include Chitin Binding Protein, Maltose Binding Protein, Glutathione-S-transferase Protein, Polyhistidine, FLAG tag, Calmodulin tag, Myc tag, BP tag, HA-tag, HA, ECS, E2, E-tag, S-tag, S1, T7, V5, VSV-G, Strep, SBP tag, Softag 1, Softag 3, V5 tag, thioredoxin (TRX), poly(NANP), tandem affinity purification (TAP) tag, Glu-Glu, HSV, KT3, Xpress tag, Green Fluorescent Protein, AcV5, AU1, AU5, Nus tag, Strep tag, Thioredoxin tag, MBP tag, VSV tag, Avi tag, biotin carboxyl carrier protein (BCCP), or any combination thereof. Other affinity tags are known in the art and described in the literature, e.g., by U.S. Pat. No. 5,750,374 and Terpe K., 2003, “Overview of Tag Protein Fusions: from molecular and biochemical fundamentals to commercial systems,” Applied Microbiology and Biotechnology (60):523-533, the entire contents of both of which are hereby incorporated by reference.

In some aspects, the RNA-binding protein comprises an affinity tag fused to the RNA-binding polypeptide. The affinity tag can be fused to the N terminus, C terminus, or internally to the RNA-binding polypeptide.

In some aspects, the affinity tag is a flag tag. In some aspects, the flag tag comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 26. In some aspects, the flag tag comprises an amino acid sequence comprising about 75% or more, at least about 85% or more, at least about 95% or more, or 100% sequence identity with a nucleic acid sequence of SEQ ID NO: 26. In some aspects, the flag tag is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 27. In some aspects, the flag tag is encoded by a nucleic acid sequence comprising about 75% or more, at least about 85% or more, at least about 95% or more, or 100% sequence identity with a nucleic acid sequence of SEQ ID NO: 27.

In some aspects, the flag tag is fused to the N terminus of the RNA-binding polypeptide. When the flag tag is fused to the N terminus of the RNA-binding polypeptide, the engineered protein further can further comprise a linker polypeptide linking the flag tag to the RNA-binding polypeptide. The linker polypeptide can comprise an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 22. In some aspects, the linker polypeptide comprises an amino acid sequence comprising about 75% or more, at least about 85% or more, at least about 95% or more, or 100% sequence identity with an amino acid sequence of SEQ ID NO: 22. In some aspects, the linker polypeptide is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 23. In some aspects, the linker polypeptide is encoded by a nucleic acid sequence comprising about 75% or more, at least about 85% or more, at least about 95% or more, or 100% sequence identity with a nucleic acid sequence of SEQ ID NO: 23.

In some aspects, the RNA-binding protein comprises an affinity tag fused to the N terminus of the RNA-binding polypeptide and further comprises a linker polypeptide linking the flag tag to the RNA-binding polypeptide. In some aspects, the RNA-binding protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 1. In some aspects, the RNA-binding protein comprises an amino acid sequence comprising about 75% or more, at least about 85% or more, at least about 95% or more, or 100% sequence identity with an amino acid sequence of SEQ ID NO: 1. In some aspects, the RNA-binding protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 2. In some aspects, the RNA-binding protein is encoded by a nucleic acid sequence comprising about 75% or more, at least about 85% or more, at least about 95% or more, or 100% sequence identity with a nucleic acid sequence of SEQ ID NO: 2.

### B. Linkers

An RNA-binding polypeptide can further comprise amino acid sequences comprising at least one linker linking the RNA-binding polypeptide to the polypeptide of interest, the protein of interest, an affinity tag, or any combination thereof. Protein linkers aid fusion protein design by providing appropriate spacing between domains, supporting correct protein folding in the case that N or C termini interactions are crucial to folding. Commonly, protein linkers permit important domain interactions, reinforce stability, and reduce steric hindrance, making them preferred for use in fusion protein design even when N and C termini can be fused. Linkers can be flexible (e.g., comprising small, non-polar (e.g., Gly) or polar (e.g., Ser, Thr) amino acids). Rigid linkers can be formed of large, cyclic proline residues, which can be helpful when highly specific spacing between domains must be maintained. In vivo cleavable linkers are designed to allow the release of one or more fused domains under certain reaction conditions, such as a specific pH gradient, or when coming in contact with another biomolecule in the cell. Examples of suitable linkers are well known in the art, and programs to design linkers are readily available (Crasto et al., Protein Eng., 2000, 13(5):3096-312), the disclosure of which is incorporated herein in its entirety. Non-limiting examples of suitable linkers include GGSGGGSGG (SEQ ID NO: 24) and (GGGGS)1-4 (SEQ ID NO: 51). Alternatively, the linker may be rigid, such as (EAAAK)1-4 (SEQ ID NO: 52), A(EAAAK)2-5A (SEQ ID NO: 53), PAPAP (AP)6-8 (SEQ ID NO: 54), and GIHGVPAA (SEQ ID NO: 55).

In some aspects, the engineered protein further comprises a flexible protein linker for linking the RNA-binding polypeptide to the protein of interest or to the affinity polypeptide. The flexible protein linker can comprise an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 24. In some aspects, the flexible protein linker comprises an amino acid linker comprising about 75% or more, at least about 85% or more, at least about 95% or more, or 100% sequence identity with SEQ ID NO: 24. In some aspects, the flexible protein linker is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 25. In some aspects, the flexible protein linker is encoded by a nucleic acid sequence comprising about 75% or more, at least about 85% or more, at least about 95% or more, or 100% sequence identity with SEQ ID NO: 25.

In some aspects, the engineered protein comprises a flag tag, a linker polypeptide linking the flag tag to the N-terminus of an RNA-binding polypeptide comprising one or more RRMs of a Bruno-like protein, a flexible protein linker linking the RNA-binding polypeptide to a protein of interest. The engineered protein can comprise an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 3. In some aspects, the engineered protein comprises an amino acid linker comprising about 75% or more, at least about 85% or more, at least about 95% or more, or 100% sequence identity with SEQ ID NO: 3. In some aspects, the engineered protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 4. In some aspects, the engineered protein is encoded by a nucleic acid sequence comprising about 75% or more, at least about 85% or more, at least about 95% or more, or 100% sequence identity with SEQ ID NO: 4.

### (e) Nucleic Acid Constructs

Expression constructs of the instant disclosure comprise a promoter operably linked to a nucleic acid sequence encoding the engineered protein. Engineered proteins can be as described above in Section I herein above.

The expression constructs can further comprise nucleic acid constructs that facilitate manipulation of the expression constructs. Non-limiting examples of suitable nucleic acid constructs include plasmid constructs, viral constructs, and self-replicating RNA (Yoshioka et al., Cell Stem Cell, 2013, 13:246-254). Non-limiting examples of suitable plasmid constructs include pUC, pBR322, pET, pBluescript, pEarlyGate, and variants thereof. Alternatively, the nucleic acid constructs can be part of a viral vector (e.g., lentiviral vectors, adeno-associated viral vectors, adenoviral vectors, and so forth).

Any of the nucleic acid constructs described herein are to be considered modular, in that the engineered protein and other components can optionally be distributed among two or more nucleic acid constructs as described herein, for instance, when an aspect of the instant disclosure comprises more than one engineered protein. The engineered proteins can be expressed from the same nucleic acid construct or can be expressed from multiple expression constructs. Similarly, when an engineered protein comprises an affinity polypeptide to tether a protein of interest to an RNA molecule, and the protein of interest is exogenously expressed, the engineered protein and the protein of interest can be expressed from the same nucleic acid construct or can be expressed from multiple expression constructs. The nucleic acid constructs can be DNA or RNA, linear or circular, single-stranded or double-stranded, or any combination thereof. The nucleic acid constructs can be codon optimized for efficient transcription and translation into protein, and possibly for transcription into an RNA donor polynucleotide transcript in the cell of interest. Codon optimization programs are available as freeware or from commercial sources. The nucleic acid constructs are introduced into a cell to express the RNA-binding polypeptide and other components in the cell. In some aspects, the nucleic acid constructs transiently express the proteins.

### A. Promoters and Other Sequences

Nucleic acid constructs for expression of a protein such as the expression constructs of the instant disclosure generally comprise DNA coding sequences operably linked to at least one promoter control sequence for expression in a cell of interest. Non-limiting examples of suitable eukaryotic promoters include constitutive, regulated, or cell- or tissue-specific promoters. Promoters can also be plant-specific promoters, or promoters that can be used in plants. A wide variety of promoters are known to those of ordinary skill in the art, as are other regulatory elements that may be used alone or in combination with promoters.

Constitutive promoters are classified as providing for a range of constitutive expression. Thus, some are weak constitutive promoters, and others are strong constitutive promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or any combination of the foregoing.

Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-3 promoter, Mb promoter, Nphsl promoter, OG-2 promoter, SP-B promoter, SYN1 promoter, and WASP promoter. Non-constitutive plant promoters include tissue-preferred promoters, tissue-specific promoters, cell-type specific promoters, and inducible-promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Examples of suitable eukaryotic regulated promoter control sequences include, without limit, those regulated by heat shock, metals, steroids, antibiotics, or alcohol. Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato. Any of the promoter sequences can be wild type or can be modified for more efficient or efficacious expression.

In some aspects, a promoter of the instant disclosure comprises a nucleic acid sequence comprising a UBQ10 promoter. In some aspects, the UBQ10 promoter comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20.

The DNA coding sequence also may be linked to a polyadenylation signal (e.g., SV40 polyA signal, bovine growth hormone (BGH) polyA signal, etc.) and/or at least one transcriptional termination sequence. In some aspects, the construct further comprises a terminator sequence. The terminator can comprise a nucleic acid sequence comprising an OCS terminator. In some aspects, the OCS terminator comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 21. In some aspects, the OCS terminator comprises a nucleic acid sequence comprising about 75% or more, at least about 85% or more, at least about 95% or more, or 100% sequence identity with a nucleic acid sequence of SEQ ID NO: 21.

The nucleic acid constructs can comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The nucleic acid constructs can further comprise RNA processing elements such as glycine tRNAs, or Csy4 recognition sites. Such RNA processing elements can, for instance, intersperse polynucleotide sequences encoding multiple proteins under the control of a single promoter to produce the multiple proteins from a transcript encoding the multiple proteins. When a cys4 recognition site is used, a vector can further comprise sequences for expression of Csy4 RNAse to process the RNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

### B. Aspects of Expression Constructs

In some aspects, the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, a nucleic acid sequence comprising a cloning sequence downstream of the nucleic acid sequence encoding the RNA-binding polypeptide, and an OCS terminator. In some aspects, the engineered protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 3. In some aspects, the engineered protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 3. In some aspects, the engineered protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the engineered protein is encoded by a nucleic acid sequence comprising about 75% or more, at least about 85% or more, at least about 95% or more, or 100% sequence identity with a nucleic acid sequence of SEQ ID NO: 4.

In some aspects, the cloning sequence comprises a nucleic acid sequence of SEQ ID NO: 19. In some aspects, the UBQ10 promoter comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20. In some aspects, the OCS terminator comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 21. In some aspects, the OCS terminator comprises a nucleic acid sequence comprising about 75% or more, at least about 85% or more, at least about 95% or more, or 100% sequence identity with a nucleic acid sequence of SEQ ID NO: 21.

In some aspects, the expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 18. In some aspects, the expression construct comprises a nucleic acid sequence comprising about 75% or more, at least about 85% or more, at least about 95% or more, or 100% sequence identity with a nucleic acid sequence of SEQ ID NO: 18.

In some aspects, the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, and a de-adenylase protein fused to the C-terminus of the RNA-binding polypeptide, and an OCS terminator.

In some aspects, the engineered protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 8. In some aspects, the engineered protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 8. In some aspects, the engineered protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 9. In some aspects, the engineered protein is encoded by a nucleic acid sequence comprising about 75% or more, at least about 85% or more, at least about 95% or more, or 100% sequence identity with a nucleic acid sequence of SEQ ID NO: 9.

In one aspect, the protein of interest is a CAF1 protein. In one aspect, the protein of interest is an Arabidopsis CAF1 protein. In some aspects, the RNA de-adenylase protein is an Arabidopsis de-adenylase protein comprising an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 6. In some aspects, the CAF1 protein comprises an amino acid sequence comprising about 75% or more, at least about 85% or more, at least about 95% or more, or 100% sequence identity with an amino acid sequence of SEQ ID NO: 6. In some aspects, the CAF1 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 7. In some aspects, the CAF1 protein is encoded by a nucleic acid sequence comprising about 75% or more, at least about 85% or more, at least about 95% or more, or 100% sequence identity with a nucleic acid sequence of SEQ ID NO: 7.

In some aspects, the cloning sequence comprises a nucleic acid sequence of SEQ ID NO: 19. In some aspects, the UBQ10 promoter comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20. In some aspects, the OCS terminator comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 21. In some aspects, the OCS terminator comprises a nucleic acid sequence comprising about 75% or more, at least about 85% or more, at least about 95% or more, or 100% sequence identity with a nucleic acid sequence of SEQ ID NO: 21.

In some aspects, the expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 16. In some aspects, the expression construct comprises a nucleic acid sequence comprising about 75% or more, at least about 85% or more, at least about 95% or more, or 100% sequence identity with a nucleic acid sequence of SEQ ID NO: 16.

In some aspects, the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, and a translation enhancer protein fused to the C-terminus of the RNA-binding polypeptide, and an OCS terminator.

In some aspects, the engineered protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 13. In some aspects, the engineered protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 13. In some aspects, the engineered protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 14. In some aspects, the engineered protein is encoded by a nucleic acid sequence comprising about 75% or more, at least about 85% or more, at least about 95% or more, or 100% sequence identity with a nucleic acid sequence of SEQ ID NO: 14.

In some aspects, the translation enhancer is an RPS6 protein. In some aspects, the RPS6 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 11. In some aspects, the RPS6 protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 11. In some aspects, the RPS6 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 12. In some aspects, the RPS6 protein is encoded by a nucleic acid sequence comprising about 75% or more, at least about 85% or more, at least about 95% or more, or 100% sequence identity with a nucleic acid sequence of SEQ ID NO: 12.

In some aspects, the cloning sequence comprises a nucleic acid sequence of SEQ ID NO: 19. In some aspects, the UBQ10 promoter comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20. In some aspects, the OCS terminator comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 21. In some aspects, the OCS terminator comprises a nucleic acid sequence comprising about 75% or more, at least about 85% or more, at least about 95% or more, or 100% sequence identity with a nucleic acid sequence of SEQ ID NO: 21.

In some aspects, the expression construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 15. In some aspects, the expression construct comprises a nucleic acid sequence comprising about 75% or more, at least about 85% or more, at least about 95% or more, or 100% sequence identity with a nucleic acid sequence of SEQ ID NO: 15.

### II. Expression Vector

Another aspect of the instant disclosure encompasses an expression vector for tethering a protein of interest to an RNA molecule. The vectors are designed to facilitate generation of engineered proteins comprising any protein of interest linked to an RNA-binding polypeptide. To achieve this, the vectors comprise an expression construct comprising a first nucleic acid sequence encoding the RNA-binding polypeptide, and a second nucleic acid sequence comprising a cloning sequence upstream or downstream of the first nucleic acid sequence, whereby a third nucleic acid sequence encoding the protein or polypeptide of interest cloned into the cloning sequence generates the nucleic acid sequence encoding the engineered protein. Engineered proteins can be as described in Section I above, and promoters can be as described in Section II(a) herein above.

Cloning sequences and methods of designing multiple cloning sites operable to clone nucleic acid sequences encoding proteins are known in the art, and generally comprise a nucleic acid sequence comprising one or more restriction sites for cloning a nucleic acid sequence. An expression vector comprising a nucleic acid sequence encoding the protein of interest cloned into the multiple cloning sequence expresses an engineered protein comprising a first terminus of the RNA-binding polypeptide fused to the protein of interest. In some aspects, the cloning sequence comprises a nucleic acid sequence of SEQ ID NO: 19.

In some aspects, the expression vector comprises a UBQ10 promoter operably linked to a nucleic acid sequence encoding an engineered protein comprising a flag tag fused to the N-terminus of an RNA-binding polypeptide comprising one or more RRMs of a Bruno-like protein, a nucleic acid sequence comprising a cloning sequence downstream of the nucleic acid sequence encoding the RNA-binding polypeptide, and an OCS terminator. In some aspects, the engineered protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 3. In some aspects, the engineered protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 3. In some aspects, the engineered protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 4. In some aspects, the engineered protein is encoded by a nucleic acid sequence comprising about 75% or more, at least about 85% or more, at least about 95% or more, or 100% sequence identity with a nucleic acid sequence of SEQ ID NO: 4.

In some aspects, the cloning sequence comprises a nucleic acid sequence of SEQ ID NO: 19. In some aspects, the UBQ10 promoter comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20. In some aspects, the OCS terminator comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 21. In some aspects, the OCS terminator comprises a nucleic acid sequence comprising about 75% or more, at least about 85% or more, at least about 95% or more, or 100% sequence identity with a nucleic acid sequence of SEQ ID NO: 21.

In some aspects, the expression vector comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 18. In some aspects, the expression construct comprises a nucleic acid sequence comprising about 75% or more, at least about 85% or more, at least about 95% or more, or 100% sequence identity with a nucleic acid sequence of SEQ ID NO: 18.

In some aspects, the expression vector comprises a nucleic acid construct comprising the expression vector. In some aspects, the nucleic acid construct comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 41. In some aspects, the plasmid comprises a nucleic acid sequence comprising about 75% or more, at least about 85% or more, at least about 95% or more, or 100% sequence identity with a nucleic acid sequence of SEQ ID NO: 41.

In some aspects, the expression vector comprises a UBQ10 promoter operably linked to a nucleic acid sequence a cloning sequence upstream of the nucleic acid sequence encoding the RNA-binding polypeptide comprising one or more RRMs of a Bruno-like protein, and an OCS terminator. In some aspects, the engineered protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with an amino acid sequence of SEQ ID NO: 33. In some aspects, the engineered protein comprises an amino acid sequence comprising about 75% or more, 85% or more, 90% or more, or about 90%, 91%, 92%, 93%, 94%, 95%, 96%, 97%, 98%, 99%, or 100% sequence identity with an amino acid sequence of SEQ ID NO: 33. In some aspects, the engineered protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 34. In some aspects, the engineered protein is encoded by a nucleic acid sequence comprising about 75% or more, at least about 85% or more, at least about 95% or more, or 100% sequence identity with a nucleic acid sequence of SEQ ID NO: 34.

In some aspects, the cloning sequence comprises a nucleic acid sequence of SEQ ID NO: 19. In some aspects, the UBQ10 promoter comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with SEQ ID NO: 20. In some aspects, the UBQ10 promoter comprises a nucleic acid sequence comprising about 75% or more, at least about 85% or more, at least about 95% or more, or 100% sequence identity with a nucleic acid sequence of SEQ ID NO: 20. In some aspects, the OCS terminator comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 21. In some aspects, the OCS terminator comprises a nucleic acid sequence comprising about 75% or more, at least about 85% or more, at least about 95% or more, or 100% sequence identity with a nucleic acid sequence of SEQ ID NO: 21.

In some aspects, the expression vector comprises a nucleic acid construct comprising the expression vector, the nucleic acid construct comprising a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with a nucleic acid sequence of SEQ ID NO: 40.

In some aspects, the expression construct comprises a nucleic acid sequence comprising about 75% or more, at least about 85% or more, at least about 95% or more, or 100% sequence identity with a nucleic acid sequence of SEQ ID NO: 40.

### III. Two or More Expression Constructs

Yet another aspect of the instant disclosure encompasses two or more expression constructs encoding an engineered system for tethering a protein of interest to a target RNA molecule. The two or more expression constructs comprise an expression construct for expressing an engineered protein comprising an RNA-binding polypeptide fused to a protein or polypeptide of interest. The two or more expression constructs also comprise an expression construct for expressing the target RNA molecule, wherein the expression construct comprises a promoter operably linked to a nucleic acid sequence encoding the target RNA molecule. The expression construct for expressing an engineered protein can be as described in Section I herein above. The target RNA molecule can be as described in Section I(c) herein above.

As explained in Section I(e) herein above, any of the nucleic acid constructs described herein are to be considered modular, in that the engineered protein and other components, such as an expression construct for expressing a target RNA molecule can optionally be on a single nucleic acid construct or can be distributed among two or more nucleic acid constructs as described herein. The engineered proteins can be expressed from the same nucleic acid construct, or can be expressed from multiple expression constructs. Similarly, when an engineered protein comprises an affinity polypeptide to tether a protein of interest to an RNA molecule, and the protein of interest is exogenously expressed, the engineered protein and the protein of interest can be expressed from the same nucleic acid construct, or can be expressed from multiple expression constructs. The nucleic acid constructs can be DNA or RNA, linear or circular, single-stranded or double-stranded, or any combination thereof. The nucleic acid constructs can be codon optimized for efficient transcription and translation into protein, and possibly for transcription into an RNA donor polynucleotide transcript in the cell of interest. Codon optimization programs are available as freeware or from commercial sources. The nucleic acid constructs are introduced into a cell to express the RNA-binding polypeptide and other components in the cell. In some aspects, the nucleic acid constructs transiently express the proteins.

### IV. Engineered Protein

An additional aspect of the instant disclosure encompasses an engineered protein for tethering a protein of interest to a target RNA molecule. The engineered protein comprises an RNA-binding polypeptide fused to a protein or polypeptide of interest, wherein the RNA-binding polypeptide comprises an RNA-binding domain of an RNA-binding protein and wherein the RNA-binding domain recognizes and specifically binds an RNA recognition sequence in the target RNA molecule to thereby tether the protein of interest to the target RNA molecule. The engineered proteins of the instant disclosure and expression construct for expressing engineered proteins can be as described in Section I herein above. The target RNA molecule can be as described in Section I(c) herein above.

### V. Cells

An additional aspect of the instant disclosure encompasses a cell comprising one or more expression constructs for expressing an engineered protein for tethering a protein of interest to a target RNA molecule, one or more nucleic acid expression vectors for tethering a protein of interest to an RNA molecule, two or more expression constructs encoding an engineered system for tethering a protein of interest to a target RNA molecule, and engineered protein, or any combination thereof. The expression constructs can be as described in Sections I-III herein above. An engineered protein can be as described in Section IV herein above.

A cell of the instant disclosure can be any cell or tissue type, or a combination of cells and tissues from or in a subject or organism. Cells can be animal, fungal, prokaryotic, and plant cells. The subject can be a human, a livestock animal, a companion animal, a lab animal, or a zoological animal. In one aspect, the subject can be a rodent, e.g. a mouse, a rat, a guinea pig, a plant, a prokaryote, etc. Non-limiting examples of suitable livestock animals can include pigs, cows, horses, goats, sheep, llamas and alpacas. Non-limiting examples of companion animals can include pets such as dogs, cats, rabbits, and birds. As used herein, a “zoological animal” refers to an animal that can be found in a zoo. Such animals can include non-human primates, large cats, wolves, and bears. Non-limiting examples of a laboratory animal can include rodents, canines, felines, and non-human primates. Non-limiting examples of rodents can include mice, rats, guinea pigs, etc. In some aspects, the subject is a human subject.

The cell may be a plant cell, a plant part, or a plant. Plant cells include germ cells and somatic cells. Non-limiting examples of plant cells include parenchyma cells, sclerenchyma cells, collenchyma cells, xylem cells, and phloem cells. Plant parts include, but are not limited to, stems, roots, ovules, stamens, leaves, embryos, meristematic regions, callus tissue, gametophytes, sporophytes, pollen, microspores, and the like. The plant can be a monocot plant or a dicot plant. For instance, the plant can be soybean; maize; sugar cane; beet; tobacco; wheat; barley; poppy; rape; sunflower; alfalfa; sorghum; rose; carnation; gerbera; carrot; tomato; lettuce; chicory; pepper; melon; cabbage; oat; rye; cotton; millet; flax; potato; pine; walnut; citrus (including oranges, grapefruit etc.); hemp; oak; rice; petunia; orchids; Arabidopsis; broccoli; cauliflower; brussel sprouts; onion; garlic; leek; squash; pumpkin; celery; pea; bean (including various legumes); strawberries; grapes; apples; cherries; pears; peaches; banana; palm; cocoa; cucumber; pineapple; apricot; plum; sugar beet; lawn grasses; maple; teosinte; Tripsacum; Coix; triticale; safflower; peanut; cassava, and olive. In some aspects, the plant is a disease-resistant cassava plant. In some aspects, the plant is a CBB-resistant cassava plant, a CBSD-resistant cassava plant, or a cassava plant resistant to CBB and CBSD.

The disclosure also provides an agricultural product produced by any of the described transgenic plants, plant parts, and plant seeds. Agricultural products include, but are not limited to, plant extracts, proteins, amino acids, carbohydrates, fats, oils, polymers, vitamins, and the like.

### VI. Methods

A further aspect of the instant disclosure encompasses a method of determining the function of a protein of interest or an RNA molecule in RNA-protein interactions. The method comprises providing or having provided one or more expression constructs for expressing an engineered protein comprising an RNA-binding polypeptide fused to a protein or polypeptide of interest and introducing the one or more constructs into a cell. Expression constructs and an engineered protein can be as described in Sections I-IV. The cell is cultured under conditions suitable for expressing the engineered protein, wherein the RNA-binding polypeptide comprises an RNA-binding domain of an RNA-binding protein and wherein the RNA-binding domain recognizes and specifically binds an RNA recognition sequence in the target RNA molecule to thereby tether the protein of interest to the target RNA molecule. The method further comprises investigating one or more phenotypes resulting from tethering the protein of interest to the target RNA molecule in the cell to thereby determine the function of the protein of interest or the target RNA molecule in RNA-protein interaction. In some aspects, the target RNA molecule is an mRNA encoding a SOC1 protein, and the phenotype is flowering time.

Another aspect of the instant disclosure encompasses a method of identifying one or more RNA-binding proteins that bind a target RNA. The method comprises expressing an engineered protein for tethering a protein of interest to a target RNA molecule in a cell comprising the target RNA molecule. The method further comprises isolating the target RNA molecule or the engineered protein and proteins bound to the target RNA molecule or the engineered protein from the cell, and identifying the proteins bound to the target RNA molecule from the isolated target RNA molecule or the protein of interest. Expression constructs and engineered proteins can be as described in Sections I-IV.

Yet another aspect of the instant disclosure encompasses a method of modifying a function of a target RNA molecule. The method comprises tethering a protein of interest to a target RNA molecule using a method described herein above, wherein tethering the protein of interest modifies the function of the target RNA molecule. When the protein of interest is a de-adenylase protein and the target RNA molecule can be an mRNA, the tethered de-adenylase protein reduces the stability of the mRNA. Conversely, when the protein of interest is a translation enhancer protein and the target RNA molecule is an mRNA, the tethered translation enhancer protein increases translation of the mRNA.

In all the methods described herein above, the methods can be performed in a cell, or can be in a cell-free in vitro system using techniques known to individuals of skill in the art.

(a) Introduction into the Cell

The methods can comprise introducing a nucleic acid construct expressing an engineered protein into a cell of interest. As explained above, an engineered protein can be encoded on more than one nucleic acid sequence. Accordingly, a method of the instant disclosure comprises introducing more than one nucleic acid construct into the cell.

The one or more nucleic acid constructs described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (b) Culturing a Cell

The method further comprises culturing a cell under conditions suitable for expressing the engineered protein. Methods of culturing cells are known in the art. In some aspects, the cell in from an animal, fungi, oomycete or prokaryote. In some aspects, the cell is a plant cell, plant, or plant part. When the cell is in tissue ex vivo, or in vivo within a plant or within a plant part, the plant part and/or plant may also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and may and will vary depending on the cell type. Routine optimization may be used, in all cases, to determine the best techniques for a particular cell type. See for example, in Santiago et al. (2008) PNAS 105:5809-5814; Moehle et al. (2007) PNAS 104:3055-3060; Urnov et al. (2005) Nature 435:646-651; Lombardo et al. (2007) Nat. Biotechnology 25:1298-1306; and Taylor et al. (2012) Tropical Plant Biology 5:127-139.

### VII. Kits

A further aspect of the instant disclosure provides kits comprising one or more expression constructs for expressing an engineered protein for tethering a protein of interest to a target RNA molecule, two or more expression constructs encoding an engineered system for tethering a protein of interest to a target RNA molecule, one or more expression vectors for tethering a protein of interest to an RNA molecule, or any combination thereof. The kits can also comprise cells comprising the expression constructs and vectors, an engineered protein and/or system for tethering a protein of interest to a target RNA molecule or any combination thereof. The expression constructs, vectors, engineered proteins and systems, and cells can be as described in Sections I to V herein above.

The kits may further comprise transfection reagents, cell growth media, selection media, in-vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the instant disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” cell refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the instant disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or any combination thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “recognition sequence” or “response element” refer to a nucleic acid sequence that defines a portion of a nucleic acid sequence recognized and can be specifically bound by an engineered protein of the instant disclosure.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

The terms “tether” and “tethering” refer to the covalent or noncovalent bonding of two or more items using synthetic biologic approaches. This includes the recruitment of an affinity tag or protein of interest to an RNA of interest, mediated by one or more RNA-binding polypeptides.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the instant disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the instant disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Introduction

The sorting of RNA transcripts dictates their ultimate post-transcriptional fates, such as translation, decay or degradation by RNA interference (RNAi). This sorting of RNAs into distinct fates is mediated by their interaction with RNA-binding proteins. While hundreds of RNA-binding proteins have been identified, the identity of proteins that act to sort RNAs into different pathways is largely unknown. Particularly in plants, this is due to the lack of reliable protein-RNA artificial tethering tools necessary to determine the mechanism of protein action on an RNA in vivo. The examples herein describe a protein-RNA tethering system generated by the inventors which functions on an endogenous Arabidopsis RNA that is easily tracked by the quantitative flowering time phenotype. Unlike other protein-RNA tethering systems that have been attempted in plants, the system described herein circumvents the inadvertent triggering of RNAi. Using the system, the inventors successfully tethered in vivo a protein epitope, a de-adenylase protein, and a translation factor to the RNA, which function to tag, decay, and boost protein production, respectively. The inventors demonstrated that the tethering system 1) is sufficient to engineer the downstream fate of an RNA, 2) enables the determination of the function of any protein upon recruitment to an RNA, 3) can be used on different RNAs, and 4) can be used to discover new interactions with RNA-binding proteins.

Plant genomes encode hundreds of proteins that interact with and regulate RNA. However, the roles of these proteins in post-transcriptional gene regulation remain widely unknown, in part due to the lack of experimental tools to study their function. For example, it is not understood which proteins are sufficient for the key regulatory decision that directs an RNA transcript to enter either the RNA decay or RNA interference (RNAi) pathway. This decision point is critical, as decay will only remove one RNA transcript, while the positive feedback cycle of RNAi carries the fate of continued degradation of additional RNA molecules through the production of small interfering RNAs (siRNAs). Artificially recruiting a protein of interest to a known RNA in vivo (protein-RNA tethering) is an essential technique for deciphering the function of RNA-binding proteins. Once artificially forced to a reporter RNA, the unknown function of the protein on that RNA can be assessed by standard RNA and protein biology techniques. Systems such as bacteriophage MS2 (MS2 protein binds an RNA sequence called the MS2 stem-loop) and AN (AN protein binds an RNA sequence called box B) have been used in yeast, Drosophila and other systems to tether a protein to a reporter RNA in order to study mRNA stability, splicing, localization, transport and translation. More recently, a CRISPR/Cas system was discovered that uses a CRISPR guide RNA (gRNA) to program the targeting of the Cas13 protein to an RNA, rather than the typical DNA target of Cas9. Protein-RNA tethering can be accomplished by synthetically fusing Cas13 to any protein of interest to investigate the function of that protein on the RNA.

Plants are highly sensitive to the production of double-stranded RNA (dsRNA). Whether it is via transcription through an inverted repeat (forming an intramolecular hairpin), the pairing of complementary transcripts (intermolecular interaction), or produced by an RNA-dependent RNA Polymerase (RDR) protein, dsRNA is a trigger for RNA cleavage by the DICER family proteins. This cleavage produces either a single small RNA molecule (microRNA) or if the dsRNA is longer, a series of siRNAs, both of which are able to trigger post-transcriptional gene silencing (PTGS) of complementary mRNA transcripts. In some cases, the cleaved target mRNA is further converted into dsRNA by an RDR protein and produces secondary siRNAs in the cycle of RNAi, amplifying the PTGS and resulting in significant reduction of complementary mRNAs and their encoded proteins.

Existing protein-RNA tethering systems are not well-developed in plants because they each trigger the plant's sensitive dsRNA response. In the case of the MS2 and AN systems, they both require the target RNA to be transgenic in order to carry the necessary MS2 stem-loop and box B binding sites. The hairpin dsRNA secondary structure of these binding sites closely resembles stem-loop structures normally processed by DICER family proteins. In plants, use of these MS2 stem-loop and box B binding sites complicates downstream analyses, as transgenic reporter RNAs are often subject to PTGS even without protein tethering. Cas13 systems of protein-RNA tethering can overcome this problem, as they can target any endogenous RNA and are not dependent on the formation of intramolecular dsRNA. However, CRISPR gRNAs need to be complementary to their target RNA and generate 28-30 nucleotide (nt) intermolecular dsRNA, which is known in plants to trigger PTGS of the target RNA even without the presence of the Cas13 protein. Therefore, each of the existing in vivo systems of protein-RNA tethering trigger the plant's sensitive response to dsRNA, degrading the target RNA independently of protein binding or action. In order to identify new RNA-binding proteins and characterize their function, the inventors aimed to generate a plant in vivo protein-RNA tethering system in which the target RNA is stable and not subject to PTGS. The protein-RNA tethering system described herein can act on an endogenous (non-transgenic) RNA without intramolecular or intermolecular dsRNA formation, and consequently does not spontaneously trigger PTGS.

### Example 2. A Minimal Version of the BRN1 Protein Retains SOC1 RNA-Binding

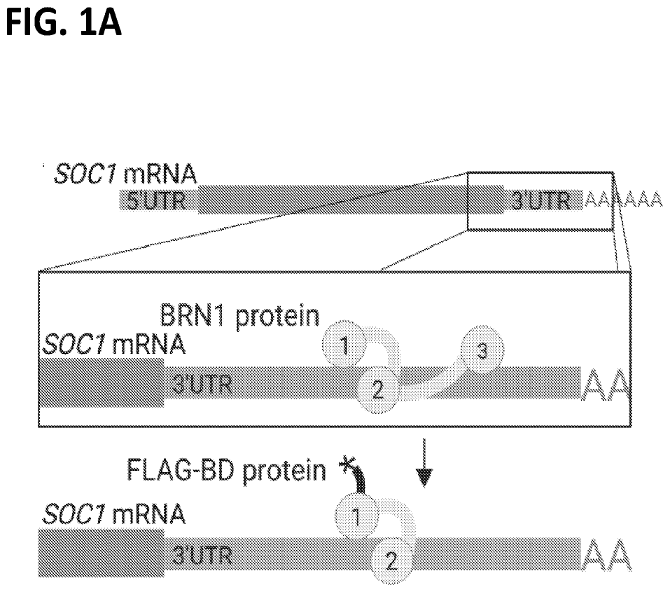

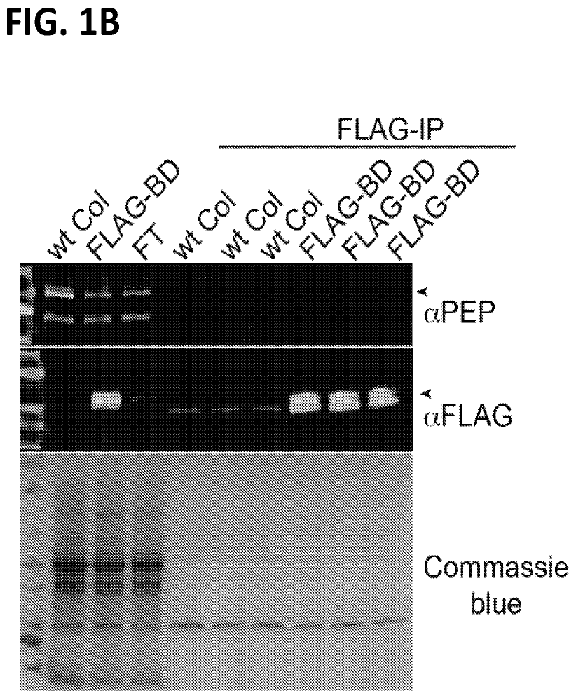

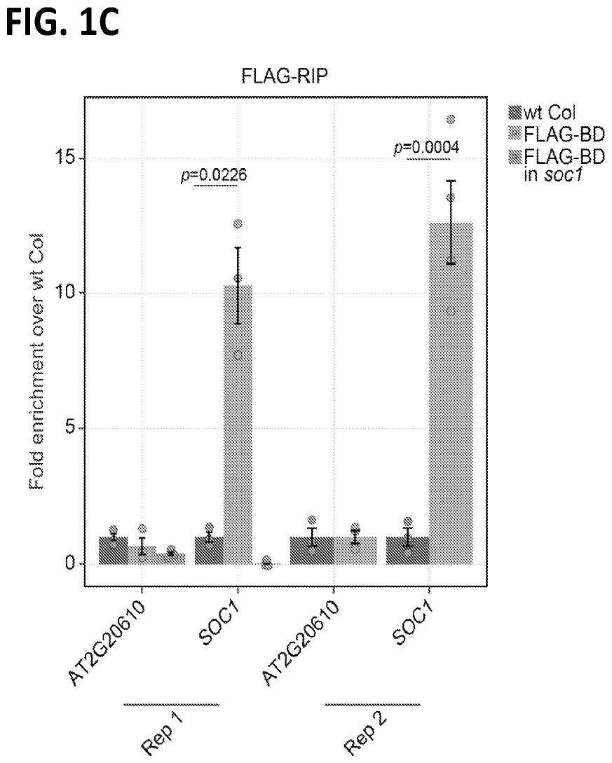

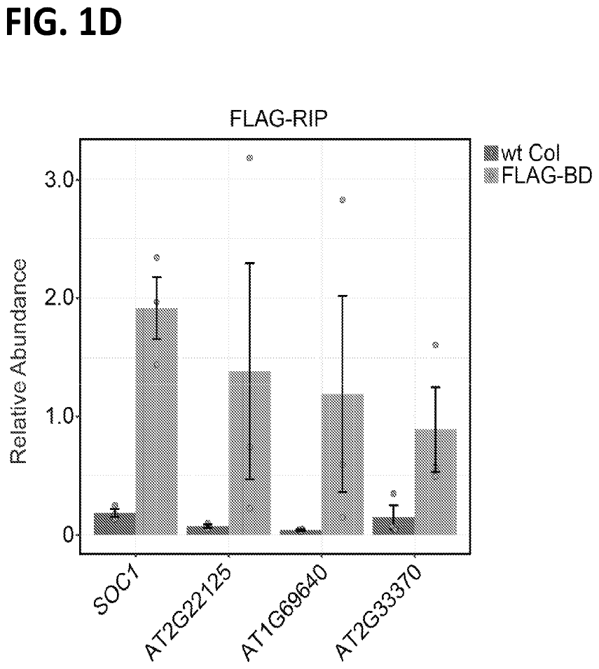

Bruno-like proteins are deeply conserved RNA-binding proteins. In Drosophila, Bruno binds a repeated 7-nt sequence in the 3′ UTR of the Oskar mRNA. In Arabidopsis thaliana, the Bruno ortholog Bruno-like 1 (BRN1) binds a single 7-nt sequence (5′UAUGUAU; SEQ ID NO: 5) in the 3′UTR of the SOC1 mRNA (FIG. 1A) and limits SOC1 translation. SOC1 is a known integrator of flowering time cues, as soc1 mutants flower late and brn1 mutants have the opposite effect of higher accumulation of SOC1 protein and early flowering. Although Bruno-like proteins characteristically have three RNA Recognition Motif (RRM) domains (domains 1-3, FIG. 1A), across several species only the first two RRM binding domains (BDs) are necessary for mRNA interaction and specificity. The inventors generated a FLAG-tagged minimal Arabidopsis BRN1 protein that contains only the first two RRM domains (named ‘FLAG-BD’, FIG. 1A), excluding the unnecessary third RRM and the region that putatively functions to inhibit SOC1 translation. FLAG-BD was germinally transformed into wild-type (wt Col) plants and confirmed its protein production (FIG. 1B). FLAG-BD protein was then immunoprecipitated in three biological replicates (FIG. 1B) and performed an RNA-immunoprecipitation (RIP) experiment (FIG. 1B) followed by qRT-PCR. In two distinct experiments with different biological replicates, a 10.6-fold and 12.5-fold enrichment of the SOC1 mRNA was observed when compared to plants without FLAG-BD (FIG. 1C). In addition, as a control, SOC1 mRNA was not detected in the RIP of FLAG-BD in soc1 mutant plants (FIG. 1C). This experiment confirms that the minimal FLAG-BD protein retains the ability to bind the endogenous SOC1 mRNA.

In addition to SOC1, there are 2,236 other mRNAs in the Arabidopsis transcriptome that have the identical 7-nt BRN1 binding site in their 3′UTR. To test if FLAG-BD also binds these RNAs, 3 mRNAs that are similarly expressed as the SOC1 mRNA in the leaf tissue were examined. It was observed that FLAG-BD binds these other mRNAs in addition to the SOC1 mRNA (FIG. 1D), likely similar to the endogenous BRN1's binding to many mRNAs that contain the 7-nt binding site.

### Example 3. Protein Tethering Itself does not Trigger PTGS or Alter Regulation of the SOC1 RNA

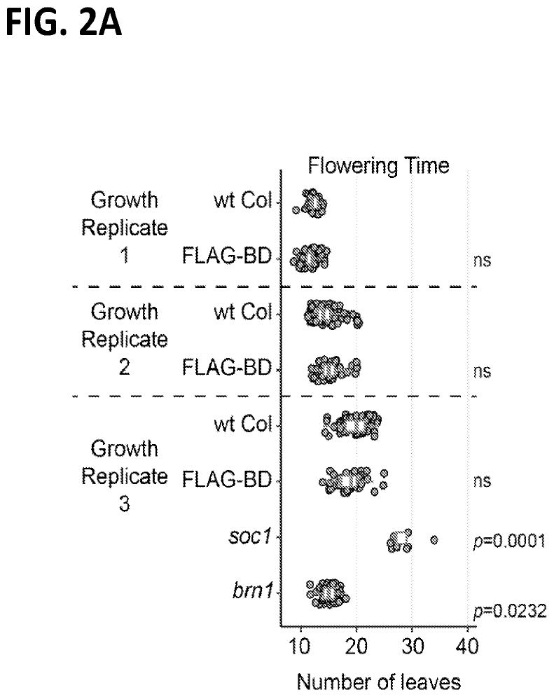

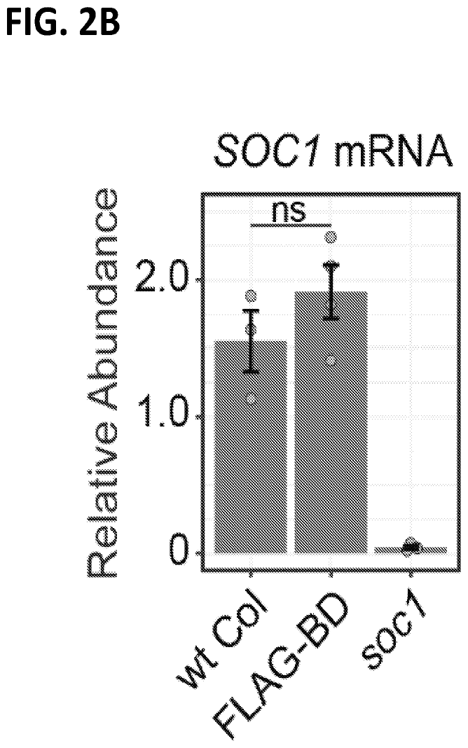

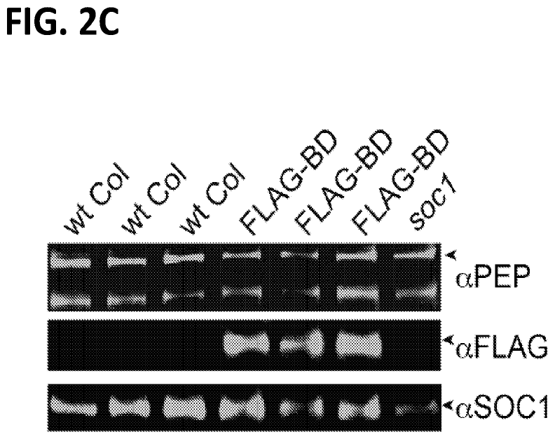

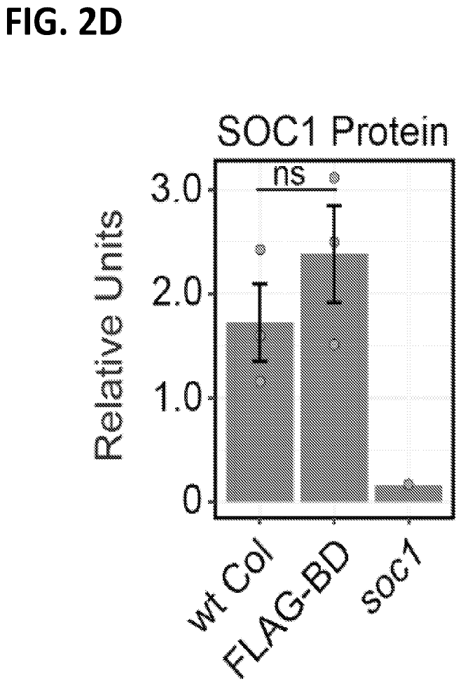

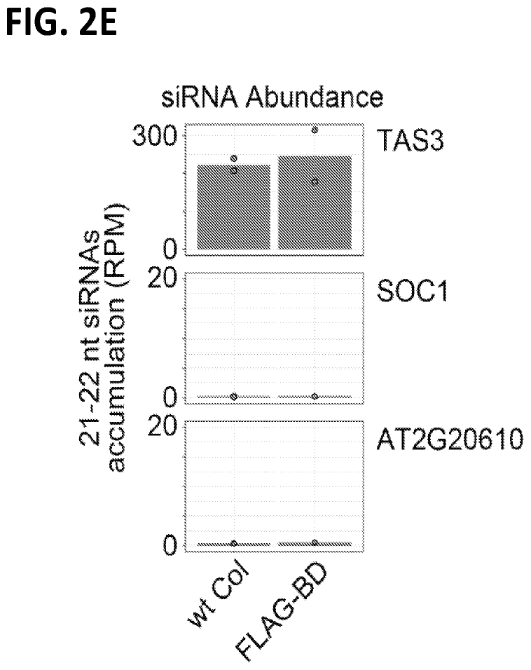

Using multiple lines of evidence, it was found that the binding of the FLAG-BD protein does not impact SOC1 regulation. In three growth replicates, FLAG-BD plants flower at the same time as plants without the FLAG-BD transgene, while soc1 and brn1 mutants flower late and early, respectively (FIG. 2A). If the FLAG-BD protein triggered PTGS of SOC1, a reduction in SOC1 mRNA and protein levels would be expected. Instead, it was found that SOC1 mRNA (FIG. 2B) and protein levels (FIG. 2C and FIG. 2D) are not decreased in plants with FLAG-BD. A small increase in the level of SOC1 mRNA and protein in FLAG-BD plants was observed, but this increase was not statistically significant (FIGS. 2B-2D). Importantly, FLAG-BD tethering does not trigger PTGS of the SOC1 mRNA, evidenced by the lack of SOC1 siRNA production assayed by small RNA sequencing (FIG. 1E). Therefore, FLAG-BD can be used as a protein-RNA tethering system to the endogenous SOC1 mRNA, eliminating the issues from techniques previously developed outside of and moved into plants.

### Example 4. Using FLAG-BD to Discover New RNA-Binding Proteins

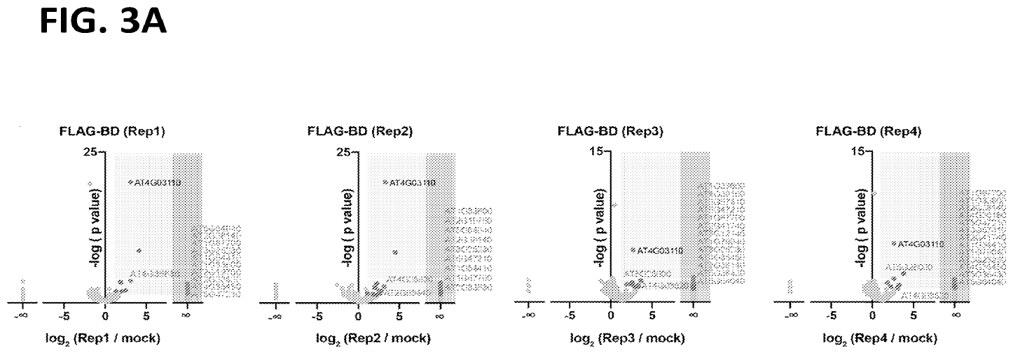

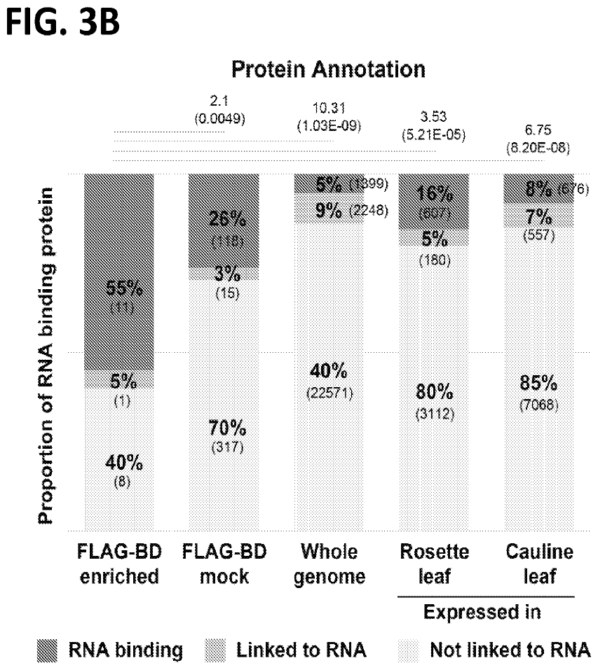

To take advantage of the interaction between the epitope-tagged FLAG-BD and its target RNAs, it was determined if FLAG-BD could be used to identify new interactions with RNA-binding proteins. Four biological replicate immunoprecipitations (IPs) of FLAG-BD plants were performed with anti-FLAG bound beads or a mock negative control with beads but no linked antibody. The samples were subjected to LC-coupled Mass Spectrometry. As expected, abundant spectra for the portions of the BRN1 protein that compose FLAG-BD were found (green points, FIG. 3A). A number of other significantly enriched proteins were also identified (blue and red points, FIG. 3A). Twenty proteins that were significantly enriched in at least 2 of the 4 biological replicates were analyzed (blue points, FIG. 3A). The identity of these 20 proteins is provided in Table 1. Eleven of the twenty proteins were previously designated as RNA-binding proteins or linked to RNA, which represents a significant enrichment compared to the mock immunoprecipitation sample, the entire genome, or the predicted proteome of the leaf tissue that was examined (FIG. 3B). The identity of the eleven RNA-binding proteins, and their weighted spectral count in each biological replicate, are listed in Table 2. This data demonstrates that the FLAG-BD tethering system can be used to identify new proteins that were previously unknown to interact during BRN1-RNA binding

TABLE 1

| | | GO category in FIG. | | | | |
|---|---|---|---|---|---|---|
| | | 3B (1: RNA binding, 2: | | | | |
| FLAG-BD | | Linked to RNA biology, | Weighted spectral Count | | | |
| Locus | Description | 3: Not linked to RNA) | Mock | Replicate 1 | Replicate 2 | Replicate 3 |
| AT1G54410 | Dehydrin family protein | 1 | 0 | 6 | 7 | 6 |
| AT1G67700 | Multidrug resistance protein | 1 | 0 | 8 | 6 | 12 |
| AT1G79040 | Photosystem Il subunit R (PSBR) | 1 | 0 | 4 | 7 | 10 |
| AT2G19750 | Ribosomal protein 530 family protein | 1 | 0 | 5 | 12 | 8 |
| AT2G33530 | Serine carboxypeptidase-like 46 (5CPL46) | 1 | 0 | 5 | 6 | 5 |
| AT2G38140 | Plastid-specific ribosomal protein 4 (PSRP4) | 1 | 0 | 9 | 10 | 9 |
| AT2G41740 | Villin 2(VLN2) | 1 | 0 | 3 | 6 | 13 |
| AT3G57410 | Villin 3(VLN3) | 1 | 0 | 2 | 9 | 13 |
| AT4G30160 | Villin 4(VLN4) | 1 | 0 | 4 | 8 | 16 |
| AT5G38430 | Ribulose bisphosphate carboxylase (small chain) | 1 | 0 | 0 | 0 | 71 |
| | family protein (RBC51B) | | | | | |
| AT5G47210 | Hyaluronan / mRNA binding family | 1 | 0 | 6 | 10 | 13 |
| AT5G26000 | thioglucoside glucohydrolase 1 (TGG1) | 2 | 29 | 25 | 47 | 112 |
| AT3G25230 | Rotamase FKBP 1 (ROF1) | 3 | 0 | 7 | 10 | 10 |
| AT4G35450 | Ankyrin repeat-containing protein 2 (AKR2) | 3 | 0 | 5 | 4 | 10 |
| AT4G35630 | Phosphoserine aminotransferase (PSAT) | 3 | 2 | 17 | 18 | 7 |
| AT5G64040 | Photosystem I reaction center subunit PSI-N | 3 | 0 | 10 | 12 | 9 |
| AT2G03440 | Nodulin-related protein 1 (NRP1) | 3 | 3 | 12 | 17 | 10 |
| AT1G33600 | Leucine-rich repeat (LRR) family protein | 3 | 0 | 5 | 14 | 17 |
| AT3G12145 | Leucine-rich repeat (LRR) family protein | 3 | 0 | 2 | 8 | 11 |
| AT4G03520 | Thioredoxin superfamily protein (ATHM2) | 3 | 2 | 3 | 9 | 21 |

| | | Weighted spectral | | | | |
|---|---|---|---|---|---|---|
| FLAG-BD | | Count | Replicate 1/ Mock | | Replicate 2/ Mock | |
| Locus | Description | Replicate 4 | Log2(FC) | NegLog(p) | Log2(FC) | NegLog(p) |
| AT1G54410 | Dehydrin family protein | 9 | INF | 1.8609699 | INF | 1.6587048 |
| AT1G67700 | Multidrug resistance protein | 14 | INF | 2.481909 | INF | 1.4216145 |
| AT1G79040 | Photosystem Il subunit R (PSBR) | 9 | INF | 1.240339 | INF | 1.6587048 |
| AT2G19750 | Ribosomal protein 530 family protein | 8 | INF | 1.550616 | INF | 2.8448184 |
| AT2G33530 | Serine carboxypeptidase-like 46 (5CPL46) | 8 | INF | 1.550616 | INF | 1.4216145 |
| AT2G38140 | Plastid-specific ribosomal protein 4 (PSRP4) | 13 | INF | 2.7924943 | INF | 2.3702402 |
| AT2G41740 | Villin 2(VLN2) | 11 | INF | 0.93013895 | INF | 1.1845685 |
| AT3G57410 | Villin 3(VLN3) | 11 | INF | 0.6200158 | INF | 2.1330175 |
| AT4G30160 | Villin 4(VLN4) | 14 | INF | 0.93013895 | INF | 1.6587048 |
| AT5G38430 | Ribulose bisphosphate carboxylase (small chain) | 77 | NaN | 0 | NaN | 0 |
| | family protein (RBC51B) | | | | | |
| AT5G47210 | Hyaluronan / mRNA binding family | 12 | INF | 1.8609699 | INF | 2.3702402 |
| AT5G26000 | thioglucoside glucohydrolase 1 (TGG1) | 104 | −0.2141248 | 0.4001247 | 0.6966078 | 0.544525 |
| AT3G25230 | Rotamase FKBP 1 (ROF1) | 9 | INF | 2.171401 | INF | 2.3702402 |
| AT4G35450 | Ankyrin repeat-containing protein 2 (AKR2) | 9 | INF | 1.550616 | INF | 0.9475666 |
| AT4G35630 | Phosphoserine aminotransferase (PSAT) | 7 | 3.087463 | 3.583188 | 3.169925 | 2.679162 |
| AT5G64040 | Photosystem I reaction center subunit PSI-N | 9 | INF | 3.1031566 | INF | 2.8448184 |
| AT2G03440 | Nodulin-related protein 1 (NRP1) | 11 | 2 | 1.8431681 | 2.5025003 | 1.9995874 |
| AT1G33600 | Leucine-rich repeat (LRR) family protein | 14 | INF | 1.550616 | INF | 3.3195734 |
| AT3G12145 | Leucine-rich repeat (LRR) family protein | 6 | INF | 0.6200158 | INF | 1.895839 |
| AT4G03520 | Thioredoxin superfamily protein (ATHM2) | 17 | 0.5849625 | 0.31794646 | 2.169925 | 1.0272644 |

| FLAG-BD | | Replicate 3/ Mock | | Replicate 4/ Mock | |
|---|---|---|---|---|---|
| Locus | Description | Log2(FC) | NegLog(p) | Log2(FC) | NegLog(p) |
| AT1G54410 | Dehydrin family protein | INF | 0.8778598 | INF | 1.4481062 |
| AT1G67700 | Multidrug resistance protein | INF | 1.7563161 | INF | 2.2533123 |
| AT1G79040 | Photosystem Il subunit R (PSBR) | INF | 1.4634309 | INF | 1.4481062 |
| AT2G19750 | Ribosomal protein 530 family protein | INF | 1.1706122 | INF | 1.2871252 |
| AT2G33530 | Serine carboxypeptidase-like 46 (5CPL46) | INF | 0.7315084 | INF | 1.2871252 |
| AT2G38140 | Plastid-specific ribosomal protein 4 (PSRP4) | INF | 1.3170133 | INF | 2.092231 |
| AT2G41740 | Villin 2(VLN2) | INF | 1.7563161 | INF | 1.6091073 |
| AT3G57410 | Villin 3(VLN3) | INF | 1.9027834 | INF | 1.7701284 |
| AT4G30160 | Villin 4(VLN4) | INF | 2.195768 | INF | 2.092231 |
| AT5G38430 | Ribulose bisphosphate carboxylase (small chain) | INF | 1.3170133 | INF | 1.4481062 |
| | family protein (RBC51B) | | | | |
| AT5G47210 | Hyaluronan / mRNA binding family | INF | 1.9027834 | INF | 1.9311696 |
| AT5G26000 | thioglucoside glucohydrolase 1 (TGG1) | 1.9493738 | 1.725202 | 1.8424587 | 1.9208927 |
| AT3G25230 | Rotamase FKBP 1 (ROF1) | INF | 1.4634309 | INF | 1.4481062 |
| AT4G35450 | Ankyrin repeat-containing protein 2 (AKR2) | INF | 1.4634309 | INF | 1.4481062 |
| AT4G35630 | Phosphoserine aminotransferase (PSAT) | 1.8073549 | 0.30037212 | 1.8073549 | 0.35879844 |
| AT5G64040 | Photosystem I reaction center subunit PSI-N | INF | 1.3170133 | INF | 1.4481062 |
| AT2G03440 | Nodulin-related protein 1 (NRP1) | 1.7369655 | 0.33240825 | 1.8744692 | 0.4858758 |
| AT1G33600 | Leucine-rich repeat (LRR) family protein | INF | 2.4888191 | INF | 2.2533123 |
| AT3G12145 | Leucine-rich repeat (LRR) family protein | INF | 1.6098652 | INF | 0.9652235 |
| AT4G03520 | Thioredoxin superfamily protein (ATHM2) | 3.3923175 | 1.6605526 | 3.087463 | 1.4150401 |

TABLE 2

| | Weighted Spectral Count | | | | |
|---|---|---|---|---|---|
| Locus | Mock | Rep 1 | Rep 2 | Rep 3 | Rep 4 |
| AT1G54410 | Dehydrin family protein | | | | |
| | 0 | 6 | 7 | 6 | 9 |
| AT1G67700 | Multidrug resistance protein | | | | |
| | 0 | 8 | 6 | 12 | 14 |
| AT1G79040 | Photosystem II subunit R (PSBR) | | | | |
| | 0 | 4 | 7 | 10 | 9 |
| AT2G19750 | Ribosomal protein S30 family protein | | | | |
| | 0 | 5 | 12 | 8 | 8 |
| AT2G33530 | Serine carboxypeptidase-like 46 (SCPL46) | | | | |
| | 0 | 5 | 6 | 5 | 8 |
| AT2G38140 | Plastid-specific ribosomal protein 4 (PSRP4) | | | | |
| | 0 | 9 | 10 | 9 | 13 |
| AT2G41740 | Villin 2 (VLN2) | | | | |
| | 0 | 3 | 6 | 13 | 11 |
| AT3G57410 | Villin 3 (VLN3) | | | | |
| | 0 | 2 | 9 | 13 | 11 |
| AT4G30160 | Villin 4 (VLN4) | | | | |
| | 0 | 4 | 8 | 16 | 14 |
| AT5G38430 | Ribulose bisphosphate carboxylase family protein (RBCS1B) | | | | |
| | 0 | 0 | 0 | 71 | 77 |
| AT5G47210 | Hyaluronan/mRNA-binding family | | | | |
| | 0 | 6 | 10 | 13 | 12 |

### Example 5. Synthetic Tethering of Protein Enzymatic Activity to the SOC1 mRNA

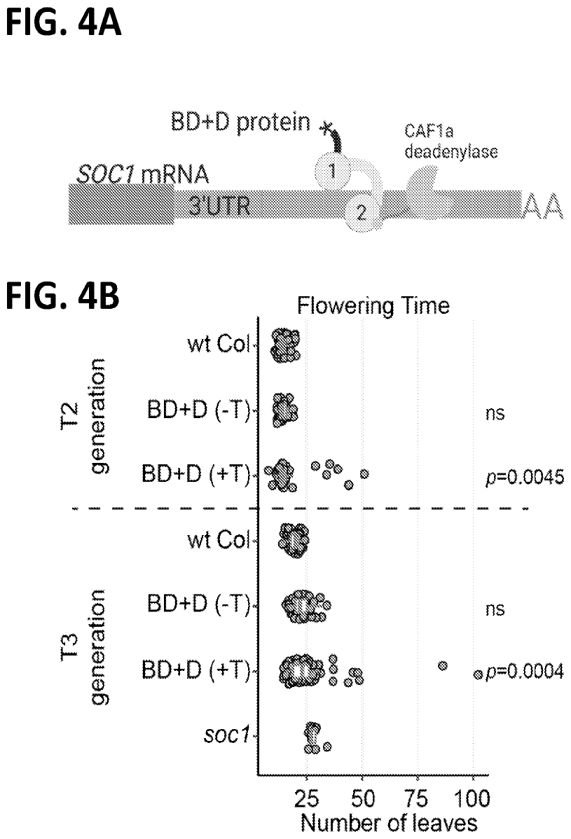

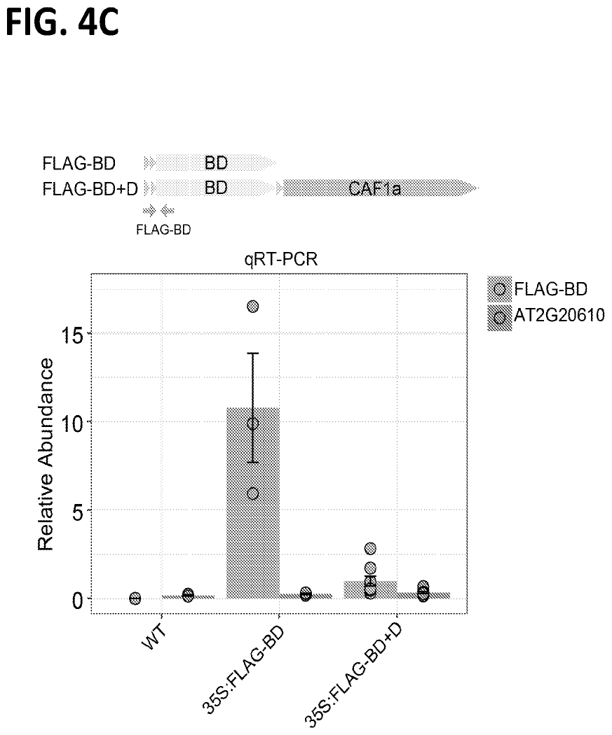

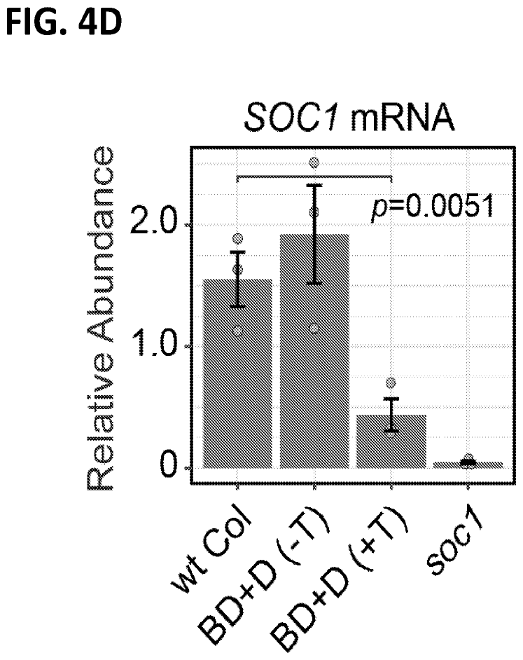

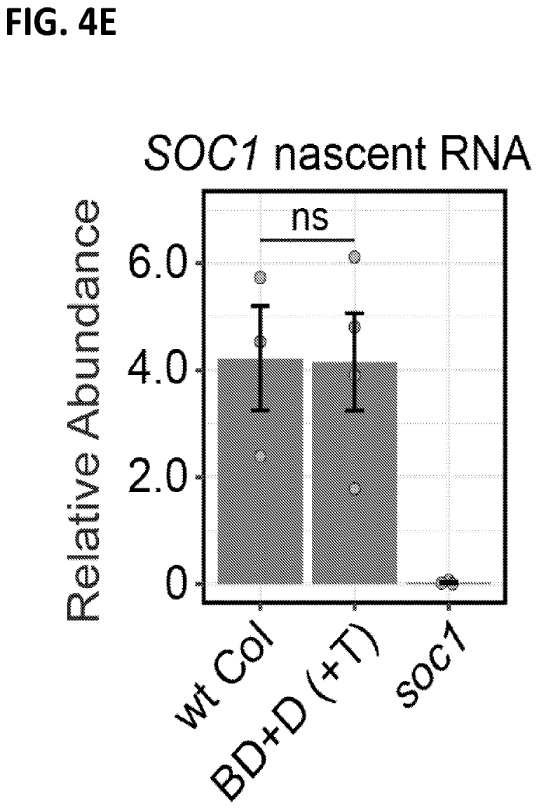

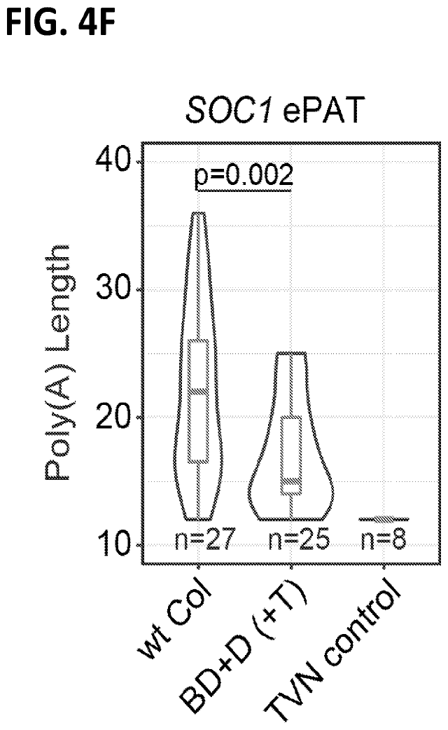

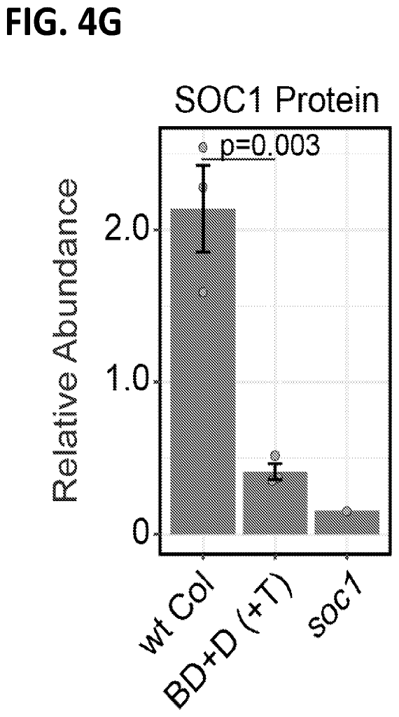

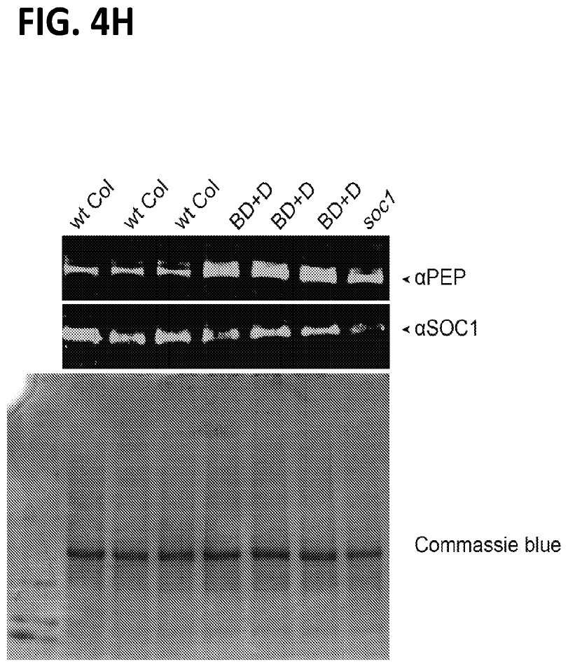

Next, it was determined if proteins (and their enzymatic functions) could be artificially tethered to the SOC1 mRNA using FLAG-BD. As a proof-of-principle, a translational fusion of the Arabidopsis CAF1a de-adenylase protein to the C-terminal end of FLAG-BD was generated, generating the ‘BD+D’ protein (FIG. 4A). The CAF1a protein removes consecutive adenosine ribonucleotides from mRNA poly(A) tails, leading to RNA decay. Consequently, a destabilization of the SOC1 mRNA and reduced protein production upon BD+D recruitment was predicted. The late flowering phenotype that corresponds to the predicted decreased levels of SOC1 was observed for 12% of second (T2) and third (T3) generation plants, while sibling plants that did not inherit the BD+D transgene do not display this phenotype (FIG. 4B). The incomplete penetrance of the late flowering phenotype in BD+D plants is due to the reduced expression of the BD+D transgene compared to FLAG-BD (FIG. 4C).

It was next determined if the BD+D fusion protein was acting directly on the SOC1 mRNA. In late flowering T2 BD+D plants, the predicted decrease in SOC1 mRNA levels was detected (FIG. 4D). This reduction is purely post-transcriptional, as the level of nascent unspliced SOC1 RNA is not altered (FIG. 4E). For these same late flowering BD+D plants, SOC1 poly(A) tail length was directly assayed by ePAT assay and Sanger sequencing of polyA tail products. It was found that late flowering BD+D plants have a shorter distribution of SOC1 mRNA poly(A) tail lengths compared to SOC1 mRNAs in wt Col (FIG. 4F). As expected, the reduced level of SOC1 mRNA and shorter poly(A) tail length corresponds to a decrease in SOC1 protein level (FIG. 4G, FIG. 4H). Together, these data demonstrate that by using the BRN1 BD, a protein can be synthetically tethered to the endogenous SOC1 mRNA, subject this mRNA to the protein's enzymatic de-adenylation activity, and is sufficient to enhance the sorting of this RNA into the RNA decay pathway.

### Example 6. Artificial Protein Tethering to an RNA can be Used to Increase Protein Production

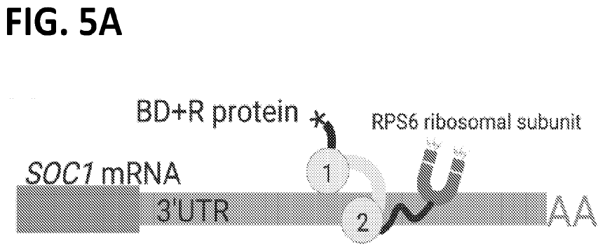

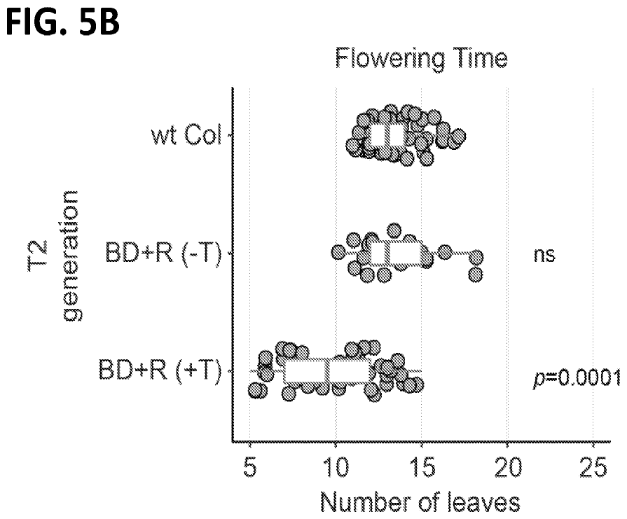

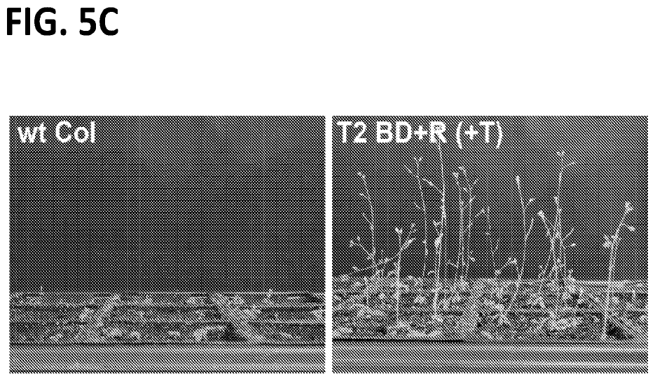

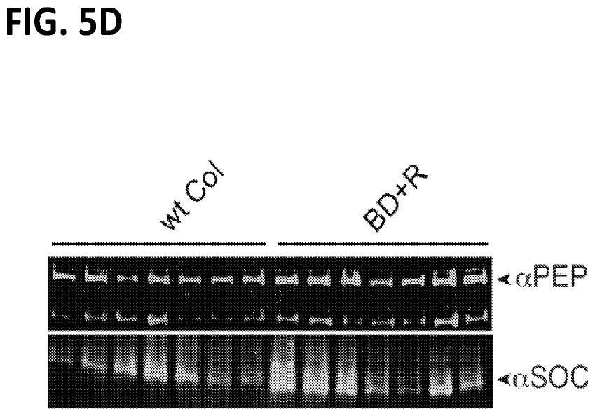

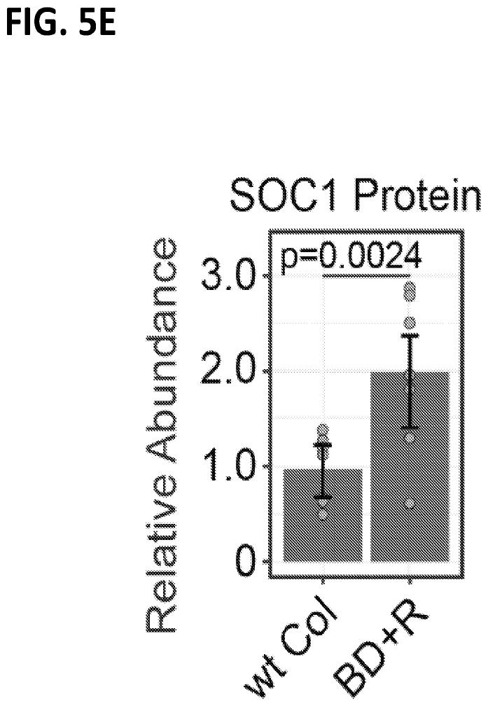

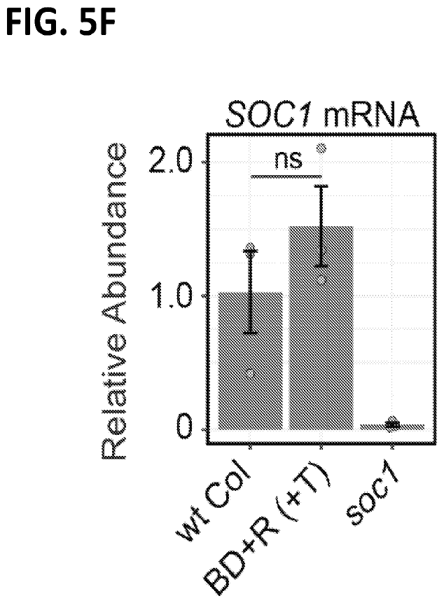

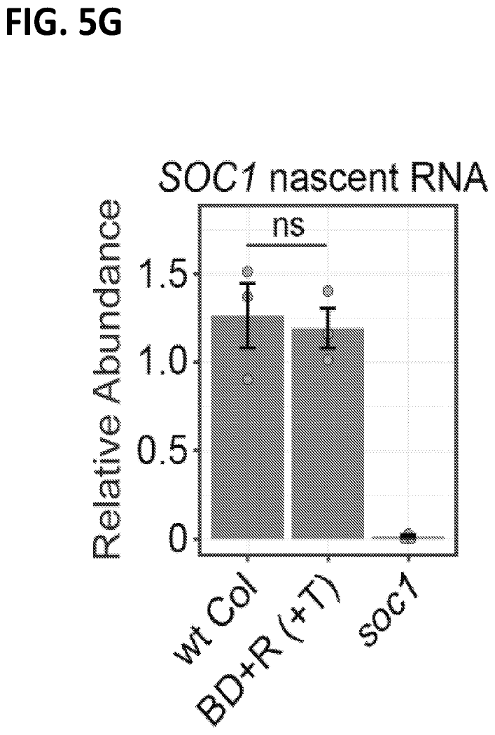

In FIGS. 4A-4H a reduction in SOC1 mRNA levels was targeted, however, the programmed destruction of RNA (knockdown) can also be accomplished by transforming a plant with an artificial microRNA or siRNA-generating construct. In contrast to targeting RNA decay and degradation, increasing RNA translation and protein production is not easily programmed. Therefore, it was aimed to tether a protein that would enhance mRNA translation and result in higher SOC1 protein level. Similar to the BD+D fusion protein, a fusion between FLAG-BD and RPS6 was generated, a conserved protein of the 40S ribosomal subunit that enhances translation in Arabidopsis (generating ‘BD+R’) (FIG. 5A). In addition, because of the poor expression of the BD+D transgene and corresponding low penetrance of the BD+D late flowering phenotype (FIG. 4B, FIG. 4C), the 35S constitutive promoter driving expression of BD was replaced with AtUBQ10 for subsequent transgenes. It was found that the BD+R construct conferred the expected early flowering phenotype in 55% of T2 plants compared to either wt Col plants or siblings that did not inherit the transgene (FIGS. 5B-C). These early-flowering plants display the expected higher accumulation of SOC1 protein (FIGS. 5D-E). This increase in SOC1 protein in BD+R lines was determined to be the result of a purely translational-level mechanism, as the level of SOC1 mRNA (FIG. 5F) and unspliced nascent RNA (FIG. 5G) are not correspondingly statistically significantly elevated. It was concluded that protein fusions to the BRN1 BD result in the successful artificial tethering of enzymatic functions to the SOC1 mRNA, and can be used to either increase or decrease SOC1 protein levels.

### Example 7. Protein tethering to any mRNA by adding the SOC1 3′UTR

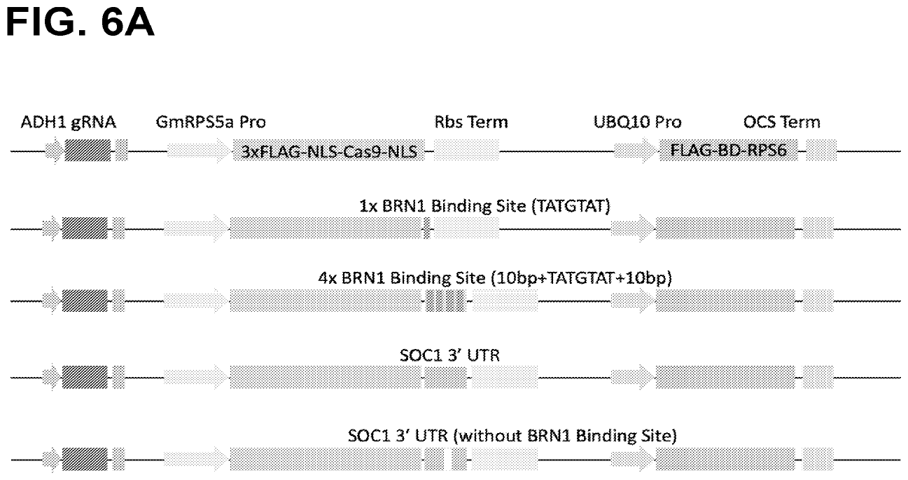

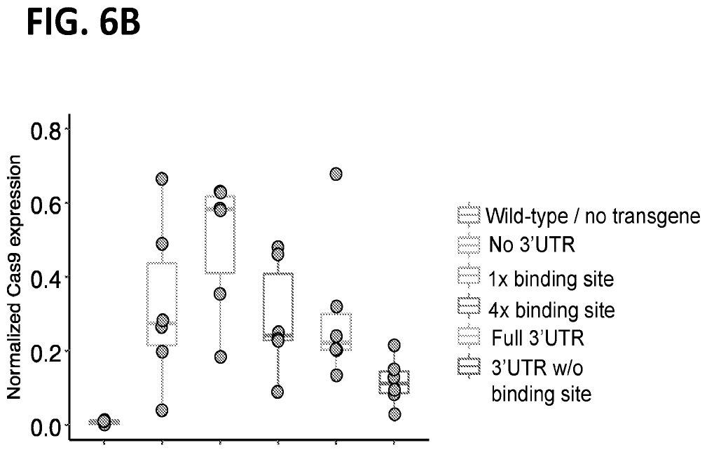

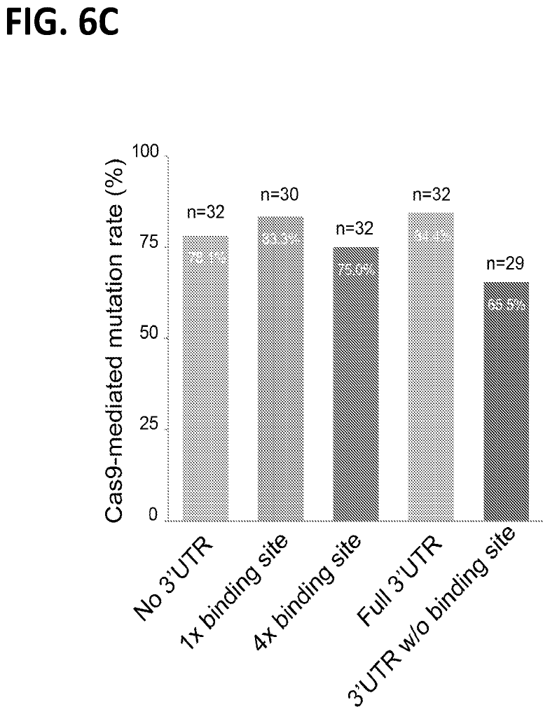

The experiments in the previous examples focused on protein-binding and regulation of the endogenous SOC1 RNA transcript since this RNA comprises the BRN1 binding site in its 3′UTR. To test if The BD binding system can be used on any RNA target, BRN1 binding site was moved to a different RNA by generating a transgene fusion. To test this system, the Cas9 RNA that encodes the Cas9 protein was used, and a gRNA that was designed based upon a previously successful gRNA that targets the ADH1 gene, with the purpose of increasing the targeting rate of ADH1 mutations using the BD-R system of translational enhancement described in FIG. 5A. Transgenes with the GmRPS5a promoter driving Cas9 with either no 3′UTR, the full SOC1 3′UTR with the BRN1 binding site, the full SOC1 3′UTR with the BRN1 binding site removed, an artificial short 3′UTR with one BRN1 binding site, and an artificial 3′UTR with an array of four BRN1 binding sites spaced out by 20 nucleotides were created (FIG. 6A). These same transgenes also encode herbicide resistance for selection, the UBQ10 Promoter:BD-R open reading frame and gRNA for ADH1 targeting (FIG. 6A). These constructs were germinally transformed into wt Col and in the T1 generation assayed Cas9 protein levels by Western blot (FIG. 6B). It was observed that the median level of Cas9 protein was highest in the construct with one BRN1 binding site, and the full SOC1 3′UTR without the BRN1 binding site had lower protein accumulation compared to this same 3′UTR with the BRN1 binding site (FIG. 6B). This demonstrates that adding the BRN1 binding site can increase the level of protein production of the artificially fused mRNA.

The ability of Cas9 to generate mutations in the transgenic lines was also measured. It was found that the rate of new mutation generation was highest in T1 plants with the transgene that has a full SOC1 3′UTR, and second highest in T1 plants with the transgene that has one copy of the BRN1 binding site transgene (FIG. 6C). Previous studies have shown that higher Cas9 expression levels leads to increased rates of gene editing, and the instant data verifies that by adding the BRN1 binding site to Cas9 in the BD-R plants, Cas9 protein levels and the rate of CRISPR/Cas9-targeted new mutations can be increased.

### Example 8. Discussion for Examples 1-7

A synthetic protein-RNA tethering system was generated that functions in vivo on an endogenous RNA, which can be easily monitored by the flowering time quantitative phenotype. Proteins were successfully and reproducibly tethered to the SOC1 mRNA, and in each instance the utility of the fused protein was demonstrated. Importantly, this system was capable of sorting the SOC1 mRNA into different fates, with CAF1a tethering leading to RNA decay and RPS6 tethering leading to enhanced translation. These proof-of-concept experiments prove that it is possible to synthetically tether a protein and enzymatic activity of the user's interest to the SOC1 mRNA. This system can be used to dissect the molecular function of any RNA-binding protein, using SOC1 as an endogenous reporter mRNA. To facilitate the fusion of any protein of interest to FLAG-BD, an AtUBQ10:FLAG-BD vector was generated with a multiple cloning site to facilitate the insertion of the user's protein(s) of interest (SEQ ID NO: 41).

Although the BRN1 BD-SOC1 mRNA tethering system overcomes a key limitation of previous protein-RNA tethering systems in plants (triggering of PTGS), there are two limitations of this system. First, there is the complicating factor of the natural biology of the endogenous BRN1 protein. The endogenous BRN1 protein likely naturally binds more than one mRNA, and we find evidence of this promiscuity in our RIP data (FIG. 1D). Therefore, in experiments such as our IP-MS (FIG. 3A), we cannot be certain that the new proteins identified interact specifically with the SOC1 mRNA. In the future, performing IP-MS with FLAG-BD in both wild-type and the soc1 mutant would resolve this specificity issue. Second, the endogenous BRN1 protein likely competes with FLAG-BD for the 7-nt binding site in the SOC1 3′UTR. We see evidence of this competition in FIG. 2C-D when the levels of SOC1 protein are slightly elevated in FLAG-BD plants. In wt Col plants the normal binding of BRN1 to the SOC1 3′UTR results in translation repression, and this repression may be blocked by FLAG-BD occupying the binding site in SOC1. If this blocking occurs, it is not enough to generate a statistically significant change in protein levels (FIG. 2D) nor a phenotypic change in flowering time (FIG. 2A). If this blocking of the BRN1 binding site is a problem in the future, this could be overcome by performing FLAG-BD experiments without competition from the endogenous BRN1 protein in a brn1 mutant background, although these plants would be expected to flower early (FIG. 2A).

### Example 9. Methods for Examples 1-8

### Plant Growth, Propagation and Collection

Arabidopsis thaliana plants of the Columbia (Col) ecotype were grown at 22° C. on Pro-Mix FPX soil in Conviron MTPS-120 growth chambers in long days (16 hours light/8 hours dark) with 200 μmol/m²/s light. Mutant alleles were described previously. Transgenic lines were transformed by the Agrobacterium-mediated floral dip method and subsequently selected with Basta herbicide. For the production of T2 and T3 generations, T1 plants were pooled and self-fertilized without selection for flowering time phenotype. Leaf tissue was collected at the time of the opening of the first flower and was used for all experiments. Biological replicates are non-overlapping pools of individuals.

### Transgene Production

The FLAG-BD and BD+D transgenes were synthesized by Integrated DNA Technologies (IDT) and cloned into pEarlyGate100 using the restriction enzymes XhoI and XbaI. Plasmids and sequences are shown in Supplemental FIG. 4.

To swap the 35S and AtUBQ10 promoters, the AtUBQ10 (AT4G05320) promoter+5′UTR sequence from pICSL12015 was directly amplified from wt Col genomic DNA using primers that contain an additional sequence for In-Fusion Cloning (Takara). pDCG006 was digested with BstBI and XhoI to remove 35S, gel purified and In-Fusion recombined with the AtUBQ10 amplicon.

To facilitate protein fusions to FLAG-BD, the ‘5′BD’ cloning vector containing an ATG codon+1× FLAG-affinity tagged minimal BRN1 protein+flexible linker sequence (no Stop codon) was synthesized via IDT, leaving the MCS that originated in pEarlyGate100 intact for future cloning of proteins to be tethered. The resulting AtUBQ10:ATG-FLAG-BD-linker-MCS plasmid is called pDCG019 (SEQ ID NO: 41).

To generate the BD+R transgene (SEQ ID NO:), RPS6 (AT4G31700) was amplified from wt Col genomic DNA using primers, and In-Fusion cloned into pDCG019 digested with BamHI and AvrII (SEQ ID NO: 41).

The plasmid series for the Cas9 tethering transgenes were modified from pHEE401 E (Addgene plasmid #71287). The 3×FLAG-NLS-zCas9-NLS and Rbs terminator were PCR amplified from pHEE401 E with primer pair YH001/YH002 and YH003/YH004, respectively. A StuI restriction enzyme cut site was introduced into the junction between 3×FLAG-NLS-zCas9-NLS and Rbs terminator, and a KpnI cut site was introduced into the end of Rbs terminator for later plasmid construction. These two PCR amplicons were assembled with NcoI/Nru/-digested linearized pYH000 plasmid using In-Fusion (Takara Bio) to create pYH001 plasmid. The pYH001a plasmid was created similarly as pYH001, with the exception of substituting primer YH003 with YH007, which leads to addition of 7-bp BRN1 binding site (TATGTAT) at the junction of 3×FLAG-NLS-zCas9-NLS and Rbs terminator. The expression cassette comprising AtUBQ10 promoter, 1×FLAG-tag, partial BRN1 coding sequence, AtRPS6 and OCS terminator was PCR amplified from previously described 5′BD-RPS6 plasmid with primer pair YH005/YH006. This amplicon was assembled with Kpn/-disgested linearized pYH001 plasmid by In-Fusion to create pYH002 plasmid, which is referred to as “No 3′UTR”. This amplicon was also assembled with Kpn/-disgested linearized pYH001a plasmid by In-Fusion to create pYH003 plasmid, which is referred to as “lx binding site”. To generate Cas9 with 4× binding sites, the 4 times tandem repeats of the 7-bp BRN1 binding site with 10-bp upstream and downstream flanking sequence (total 148-bp) was synthesized by IDT. This DNA fragment was cloned into Stu/-digested linearized pYH002 plasmid, creating pYH004 plasmid, referred to as “4× binding site”. To generate Cas9 with full SOC1 3′UTR, the SOC1 3′UTR was PCR amplified with primer pair YH008/YH009 from wild-type Col gDNA. This DNA fragment was cloned into StuI-digested linearized pYH002 plasmid, generating pYH005 plasmid, referred to as “Full 3′UTR”. The 317-bp SOC1 3′UTR without 7-bp BRN1 binding site DNA fragment was synthesized by Genewiz and cloned into Stu/-digested linearized pYH002 plasmid, generating pYH006 plasmid, refer to as “3′UTR without binding site”.

### Flowering Time Analysis

Flowering time was scored by counting the total number of rosette and cauline leaves of each plant at the time the first flower opened. Data for wt Col was collected repeatedly as it was grown side-by-side with the transgenic lines. Data was analyzed using Rstudio and plotted with ggplot2. P-value was calculated by using unpaired t-test.

### Western Blotting

Leaf tissue was grounded in liquid nitrogen and thawed in lysis buffer (50 mM Tris-HCl pH 7.5, 150 mM NaCl, 5 mM MgCl2, 10% glycerol, 1% NP-40 (IGEPAL), 0.5 mM DTT, 1 mM PMSF, 1% Plant PIC (GoldBio protease inhibitor cocktail)) and homogenized for 15 minutes at 4° C. Lysates were clarified by centrifuging for 15 minutes at 4° C. Clarified lysates were reduced and denatured by boiling in the 2× loading buffer at 95° C. for 5 minutes, and then loaded onto a 4%-20% gradient Tris-Glycine gels (BioRad). Proteins were separated at 200V for 1 hour. Protein was transferred from the gel to a PVDF membrane (Immobilon-FL, MilliporeSigma) using the BioRad semi-dry transblot for 35 minutes. Membranes were blocked for 1 hour at room temperature in Odyssey/Intercept blocking buffer (LI-COR). Primary antibodies, which include anti-SOC1 (Agrisera), anti-PEP (Rockland), and anti-FLAG (Sigma Aldrich), were all diluted 1:2000 in Odyssey blocking buffer and incubated with blots overnight. The membranes were washed 5 times at room temperature with 1×PBS-T. The IR-800 Anti-rabbit secondary antibody (LI-COR) was diluted 1:5000 and incubated with membranes for 1 hour. Membranes were washed 5 times at room temperature with 1×PBS-T, and then additional 2 times with 1×PBS. Blots were visualized using the Azure Sapphire Biomolecular Imager with exposure times ranging from 5 seconds to 5 minutes.

For the Cas9 Western blots, the PVDF membranes were blocked for 1 hour at room temperature in Azure Fluorescent Blot Blocking Buffer (Azure). The primary antibody anti-Actin (Agrisera, AC164 111) and anti-Cas9 (Diagenode, C15310258) was diluted 1:2000 and 1:5000 in the Azure blocking buffer, respectively, and incubated with the membrane over two nights. The membranes were washed 5 times at room temperature with 1×PBS-T. The anti-rabbit IR800 (Azure, AC2134) and anti-mouse IR800 (Azure, AC2135) secondary antibodies were diluted 1:2500 and incubated with membranes for 1 hour at room temperature. Membranes were washed 4 times at room temperature with 1×PBS-T, and then 1 time with 1×PBS. Blots were visualized using the Azure Sapphire Biomolecular Imager.

### Protein Quantification

Digital images of Western blots were analyzed with ImageJ for relative pixel intensities. Non-specific background noise was subtracted from raw values. SOC1 protein quantification was calculated by the ratio of SOC1/PEP values. Biological replicates were averaged and the standard deviation was calculated using Rstudio. Significance was calculated with unpaired t-test with Welch's correction.

Signal intensity of Cas9 Western blots were analyzed with AzureSpot software. Automatic background correction was done using the rolling-ball method, with rolling ball radius set as 200. Cas9 protein quantity was calculated by dividing the value of Cas9 bands by its corresponding Actin band value to normalize variability in protein loading. Five biological replicates of wild-type and six biological replicates of each transgenic line were plotted with ggplot2 in RStudio. Significance was calculated with unpaired t-test with Welch's correction.

### Protein Immunoprecipitation

Before RNA-IP, 50 μl/IP of Dynabeads Protein G (Invitrogen) were washed in 1×PBS+0.1% Tween, followed by incubation with 1 μg/IP FLAG antibody (Sigma) at room temperature for 90 minutes with rotation. For each sample, 0.5 g leaf tissue was crosslinked in formaldehyde and subsequently ground in liquid nitrogen. Proteins were extracted using 50 mM Tris-HCl pH 7.5, 150 mM NaCl, 5 mM MgCl2, 10% glycerol, 1% NP-40 (IGEPAL), 0.5 mM DTT, 1 mM PMSF, 1% Plant PIC (GoldBio protease inhibitor cocktail). Lysates were pre-cleared with Dynabeads Protein G (Invitrogen) with rotation for 20 minutes at room temperature. Pre-cleared lysates were then incubated with the prepared IP beads for 90 minutes at 4° C. with rotation. Beads were washed 3× in the washing buffer (50 mM Tris-HCl pH 7.5, 150 mM NaCl, 5 mM MgCl2, 0.5 mM DTT). After the final wash, 1 mL Trizol LS (Invitrogen) per sample was added, reverse crosslinking was performed at 55° C. for 5 minutes and RNA was extracted following the manufacturer's protocol.

### Immunoprecipitation and Mass Spectrometry

Leaf tissue was ground to fine powder in liquid nitrogen using mortar and pestle. The powder was suspended in lysis buffer (20 mM Tris-HCl, pH 7.5, 5 mM MgCl2, 300 mM NaCl, 10% glycerol, 0.5 mM DTT, 1 mM PMSF, 0.1% IGEPAL, and 1% plant protease inhibitor (GoldBio)), then centrifuged for 10 minutes at 14,000 rpm at 4° C. The supernatant was incubated with 50 μl of FLAG M2 magnetic beads (Sigma) at 4° C. for 3 hours. The beads were then washed three times in cold TBS. The FLAG-IP was eluted twice with 50 μl of 0.1 M pH2.5 glycine and neutralized with 0.5M Tris, 1.5M NaCl pH8.0 solution.

FLAG-IP elutions were reduced (10 mM TCEP) and alkylated (25 mM lodoacetamide) followed by digestion with Trypsin at 37° C. overnight. Digest was acidified with 1% TFA before cleaned up with C18 tip. The extracted peptides were dried down and each sample was resuspended in 10 μL 5% ACN/0.1% FA. 5 μL was analyzed by LC-MS with a Dionex RSLCnano HPLC coupled to an Orbitrap Fusion Lumos (Thermo Scientific) mass spectrometer using a 2 h gradient. Peptides were resolved using 75 μm×50 cm PepMap C18 column (Thermo Scientific).

All MS/MS samples were analyzed using Mascot (Matrix Science, London, UK; version 2.5.1.0). Mascot was set up to search against the provided sequences and the TAIR10 database. The digestion enzyme was set as trypsin. Mascot was searched with a fragment ion mass tolerance of 0.60 Da and a parent ion tolerance of 10 ppm. Oxidation of methionine, carbamidomethylation of cysteine, and acetylation of N-terminal of protein were specified in Mascot as variable modifications.

Scaffold (4.8.2 Proteome Software Inc., Portland, OR) was used to validate MS/MS based peptide and protein identifications. Peptide identifications were accepted if they could be established under 1% FDR by the Peptide Prophet algorithm with Scaffold delta-mass correction. Protein identifications were accepted if they could be established at greater than 99.0% probability and contained at least 2 identified peptides. Protein probabilities were assigned by the Protein Prophet algorithm. Proteins that contained similar peptides and could not be differentiated based on MS/MS analysis alone were grouped to satisfy the principles of parsimony. Proteins sharing significant peptide evidence were grouped into clusters.

### RNA Isolation

RNA was isolated using Trizol Reagent (Invitrogen) and RNA for RIP analysis was isolated using Trizol LS (Invitrogen) according to manufacturer instructions.

### Quantitative RT-PCR

5 μg of total RNA or the entire RIP RNA sample was DNase-treated using the Turbo DNA-free kit (Invitrogen). First-strand of cDNA synthesis, including the RIP RNA sample, was performed using an oligo-d(T) primer and Superscript IV reverse transcriptase (Invitrogen). For the detection of nascent RNAs, random hexamer primers were used for reverse transcription and one primer site is located in an intron. Primer sequences are shown include SEQ ID NOs: 56-81. P-value was calculated using unpaired t-tests using Welch's correction.

### Poly(A) Tail Length Determination

The length of the poly(A) tail was determined by ePAT assay. Briefly, DNase-treated RNA was ligated to the ePAT anchor primer in Superscript III buffer supplemented with RNase Out (Invitrogen) and 5U Klenow Polymerase (New England Biolabs). 200U of Superscript III (Invitrogen) was added, and the solution was reverse transcribed at 55° C. for 1 hr. The cDNA was diluted 1:6 by adding 120 μl Elution Buffer. For the ePAT TVN control reaction, instead of ePAT anchor primer, the ePAT control primer was used. For PCR amplification of cDNA, primary PCR was performed with SOC1 3′UTR II Forward primer and ePAT anchor primer. For the ePAT TVN control sample, the ePAT control primer was used in place of the ePAT anchor primer. A nested PCR was performed by diluting the primary PCR 1:100 and repeating the PCR with the primer SOC1 3′UTR III Forward and the ePAT anchor primer. Amplicons were run on a 2% high resolution agarose gel and purified. Purified amplicons were TOPO TA cloned into pCR4-TOPO (ThermoFisher) and transformed into E. coli. Individual E. coli colonies were Sanger sequenced (Eton BioScience) and poly(A) tail length was analyzed in Rstudio. Primer sequences are shown include SEQ ID NOs: 56-81. P-value was calculated using unpaired t-tests using Welch's correction.

### Small RNA Sequencing and Analysis

100 μg of total RNA was enriched for small RNAs using the miRVana miRNA isolation kit (Ambion). 1 μg of enriched small RNA was used for library preparation with the TruSeq Small RNA Library Preparation Kit (Illumina). Multiplexed libraries were sequenced on an Illumina HiSeq 3000 at the Genome Technology Access Center in Washington University.

After sequencing, adapters were trimmed from raw sequences using fastx toolkit, t/rRNAs were removed and small RNAs were filtered to the 18-28 nt size range using UEA small RNA Workbench tool and the small RNAs processed and normalized as described previously. Small RNAs were mapped to the Arabidopsis TAIR10 genome using Shortstack with default parameters except using the fractional-seeded guide approach for multi-mapped reads (--mmap f). Rstudio and ggplot2 were used to generate siRNA graphs.

### CRISPR-Induced Mutation Detection

Cas9-mediated mutation rate at ADH1 was determined by T7 endonuclease-based mutation detection assay (NEB) and High Resolution Melting (HRM) analysis (Applied Biosystems, MeltDoctor HRM Reagent Kit) according to the manufacturer's instructions. In brief, genomic DNA of 29-32 individual T1 plants from each transgenic line were extracted in the DNA extraction buffer (0.1M Tris pH8, 50 mM EDTA, 0.5M NaCl). Targeted ADH1 gene region was PCR amplified with the primer pair ADH1_FB and ADH1_RB with Q5 High-Fidelity DNA polymerase. After heteroduplex formation and T7 endonuclease I digestion, the PCR amplicon and digestion product were visualized by agarose gel electrophoresis. Primer pair ADH1_realtime_FA and ADH1_realtime_RA was used for the HRM experiment. The PCR reaction and protocol were set up according to the manufacturer's instructions and performed on QuantStudio 5 Real-time PCR system (Thermo Fisher). Three technical replicates were set up for each sample. HRM data were analyzed with High Resolution Melt (HRM) Analysis App web-based software (Thermo Fisher). Number of groups was set at 3. Samples grouped with wild-type Col were determined as “WT”; samples grouped with known mutant samples (previously tested with T7 endonuclease-based assay) were determined as “mutant”.

1. An expression construct for tethering a protein of interest to a target ribonucleic acid (RNA) molecule, the expression construct comprising:
a promoter operably linked to a nucleic acid sequence encoding an engineered protein, the engineered protein comprising an RNA-binding polypeptide comprising RNA recognition motifs (RRMs) A-B of a Bruno-like RNA-binding protein, wherein the RNA-binding polypeptide is fused to a protein or polypeptide of interest, wherein the nucleic acid sequence encoding the engineered protein comprises:
a. a first nucleic acid sequence encoding the RNA-binding polypeptide and a second nucleic acid sequence encoding the protein or polypeptide of interest upstream or downstream of the first nucleic acid sequence; or
b. a first nucleic acid sequence encoding the RNA-binding polypeptide, and a second nucleic acid sequence comprising a cloning sequence upstream or downstream of the first nucleic acid sequence, whereby a third nucleic acid sequence encoding the protein or polypeptide of interest cloned into the cloning sequence generates the nucleic acid sequence encoding the engineered protein;
wherein an amino acid sequence comprising an RRM C of the Bruno-like RNA-binding protein is absent from the RNA-binding polypeptide and wherein the RNA-binding polypeptide recognizes and specifically binds an RNA sequence comprising one or more Bruno-like protein binding sites in the target RNA molecule to thereby tether the protein or polypeptide of interest to the target RNA molecule.

2. (canceled)

3. The expression construct of claim 1, wherein the protein or polypeptide of interest comprises an affinity polypeptide that binds an epitope on the protein or polypeptide of interest.

4-9. (canceled)

10. The expression construct of claim 1, wherein the RNA-binding polypeptide comprises an amino acid sequence comprising about 75% or more, at least about 85% or more, at least about 95% or more, or 100% sequence identity with an amino acid sequence of SEQ ID NO: 33.

11-12. (canceled)

13. The expression construct of claim 1, wherein the Bruno-like RNA-binding protein is BRN1 and the RNA-binding polypeptide recognizes and specifically binds an RNA sequence comprising one or more BRN1 binding sites on the target RNA molecule.

14. The expression construct of claim 13, wherein the RNA sequence comprising the one or more BRN1 binding sites comprises about 75% or more, at least about 85% or more, at least about 95% or more, or 100% sequence identity with a nucleic acid sequence of SEQ ID NO: 5.

15. The expression construct of claim 1, wherein the engineered protein further comprises an amino acid sequence comprising an affinity tag comprising an amino acid sequence of a maltose binding protein tag, a polyhistidine tag, a Flag tag, a Myc tag, an HA-tag, a Nus tag, or any combination thereof.

16-27. (canceled)

28. The expression construct of claim 1, wherein the engineered protein comprises a flag tag, a linker polypeptide linking the flag tag to the N-terminus of an RNA-binding polypeptide comprising one or more RRMs of a Bruno-like protein, and a flexible protein linker linking the RNA-binding polypeptide to a protein or polypeptide of interest.

29. The expression construct of claim 28, wherein the engineered protein comprises an amino acid sequence comprising about 75% or more, at least about 85% or more, at least about 95% or more, or 100% sequence identity with an amino acid sequence of SEQ ID NO: 3.

30. (canceled)

31. The expression construct of claim 1, wherein the second nucleic acid sequence comprising the cloning sequence comprises a nucleic acid sequence of SEQ ID NO: 19.

32-36. (canceled)

37. The expression construct of claim 1, wherein the expression construct comprises a UBQ10 promoter operably linked to a nucleic acid sequence encoding an engineered protein comprising a flag tag fused to the N-terminus of an RNA-binding polypeptide comprising one or more RRMs of a Bruno-like protein, a nucleic acid sequence comprising a cloning sequence downstream of the nucleic acid sequence encoding the RNA-binding polypeptide, and an OCS terminator.

38-42. (canceled)

43. The expression construct of claim 37, wherein the expression construct comprises a nucleic acid sequence comprising about 75% or more, at least about 85% or more, at least about 95% or more, or 100% sequence identity with a nucleic acid sequence of SEQ ID NO: 18.

44. The expression construct of claim 1, wherein the protein or polypeptide of interest is an RNA de-adenylase protein, an RNA processing protein, an RNA translation regulation protein, an RNA helicase, an RNA transport protein an RNA decay protein, an RNA localization protein, a reporter protein, or any combination thereof.

45-67. (canceled)

68. The expression construct of claim 1, wherein the target RNA molecule comprises a plant SOC 1 mRNA comprising a 3′UTR comprising a nucleic acid sequence comprising about 75% or more, at least about 85% or more, at least about 95% or more, or 100% sequence identity with a nucleic acid sequence of SEQ ID NO: 37.

69-71. (canceled)

72. The expression construct of claim 1, wherein the target RNA comprises a chimeric mRNA comprising a nucleic acid sequence comprising one or more binding sites of a Bruno-like RNA-binding protein cloned in a 3′ UTR of the mRNA.

73. The expression construct of claim 72, wherein the chimeric mRNA encodes a Cas9 protein of a CRISPR/Cas programmable nucleic acid modification system.

74. (canceled)

75. The expression construct of claim 73, wherein the chimeric mRNA comprises a nucleic acid sequence comprising one binding site of a Bruno-like RNA-binding protein.

76. The expression construct of claim 73, wherein a nucleic acid sequence encoding the chimeric mRNA comprises a nucleic acid sequence comprising about 75% or more, at least about 85% or more, at least about 95% or more, or 100% sequence identity with a nucleic acid sequence of SEQ ID NO: 38.

77-80. (canceled)

81. Two or more expression constructs encoding an engineered system for tethering a protein or polypeptide of interest to a target RNA molecule, the two or more expression constructs comprising:
a. an expression construct of claim 1 for expressing an engineered protein comprising an RNA-binding polypeptide comprising RNA recognition motifs (RRMs) A-B of a Bruno-like RNA-binding protein, wherein the RNA-binding polypeptide is fused to a protein or polypeptide of interest; and
b. an expression construct for expressing the target RNA molecule, the expression construct comprising a promoter operably linked to a nucleic acid sequence encoding the target RNA molecule, wherein the target RNA molecule comprises one or more Bruno-like protein binding sites;
wherein the RNA-binding polypeptide recognizes and specifically binds an RNA sequence comprising one or more Bruno-like protein binding sites in the target RNA molecule to thereby tether the protein or polypeptide of interest to the target RNA molecule.

82-85. (canceled)

86. The two or more expression constructs of claim 81, wherein the target RNA molecule encodes a Cas9 protein of a CRISPR/Cas programmable nucleic acid modification system.

87. The two or more expression constructs of claim 86, further comprising an expression construct for expressing a guide RNA (gRNA) of the CRISPR/Cas programmable nucleic acid modification system, the expression construct comprising a promoter operably linked to a nucleic acid sequence encoding the gRNA.

88-91. (canceled)

92. An engineered protein for tethering a protein or polypeptide of interest to a target RNA molecule, the engineered protein comprising an RNA-binding polypeptide fused to a protein or polypeptide of interest, wherein the RNA-binding polypeptide comprises RNA recognition motifs (RRMs) A-B of a Bruno-like RNA-binding protein, wherein the RNA-binding polypeptide recognizes and specifically binds an RNA sequence comprising one or more Bruno-like protein binding sites in the target RNA molecule to thereby tether the protein or polypeptide of interest to the target RNA molecule, and wherein an amino acid sequence comprising RRM C of the Bruno-like RNA-binding protein is absent from the RNA-binding polypeptide.

93-102. (canceled)

103. A kit for tethering a protein or polypeptide of interest to a target RNA molecule, the kit comprising:
a. an expression construct of claim 1 for tethering a protein or polypeptide of interest to a target RNA molecule;
b. an expression construct of (a) and an expression construct for expressing the target RNA molecule, the expression construct comprising a promoter operably linked to a nucleic acid sequence encoding the target RNA molecule;
c. a cell comprising one or more expression constructs of (a) or (b);
d. or any combination of (a) to (c).
